(19) United States
(12) Patent Application Publication
(10) Pub. No.: US 2007/0026408 A1
(43) Pub. Date: Feb. 1, 2007

(54) Materials and methods for analysis of atp-binding cassette transporter gene expression

(75) Inventors: Robert C. Shipman, Mississauga (CA); David K. H. Lee, Milton (CA)

Correspondence Address: BERESKIN AND PARR, 40 KING STREET WEST, BOX 401, TORONTO, ON, M5H 3Y2, CA

(73) Assignee: NoAb BioDisccoveries Inc., Mississauga, ON (CA)

(21) Appl. No.: 10/582,982

(22) PCT Filed: Dec. 15, 2004
(86) PCT No.: PCT/CA04/02129

§ 371 Date: Jun. 15, 2006

Related U.S. Application Data
(60) Provisional application No. 60/529,082, filed on Dec. 15, 2003.

(51) Int. Cl. C12Q 1/68; C12M 3/00

(52) U.S. Cl. 435/6; 435/287.2; 977/924

(57) ABSTRACT

The invention provides materials and methods for detecting the expression of ABC transporter genes. The materials include sets of primers and PCR amplicons. The sets of primers are used to generate PCR amplicons, wherein each PCR amplicon is a unique portion of an ABC transporter gene. The methods of the invention include hybridization assays, such as DNA microarrays. Kits and assays for the detection of ABC transporter gene expression are also provided by the invention. In addition, the use of the materials and methods of the invention in drug screening assays is provided.

### FIELD OF THE INVENTION

The invention relates to materials and methods for detection of ATP-binding cassette transporter gene expression. In particular, the invention relates to primers and the resulting PCR products for detection of ABC transporter gene expression, and the use of said materials and methods in assays and kits.

### BACKGROUND OF THE INVENTION

ATP-binding cassette (ABC) transporters are one of the largest protein classes known to be involved in the trafficking of biological molecules across membranes. There are 48 different genes in humans which code for ABC transporters. The ABC transporters are classified into families based on the sequence and organization of their ATP-binding domain. Currently, there are seven families, which are designated A through G. The families are further classified into subfamilies based on their gene and protein structure.

All of the 48 human genes encoding the ABC transporters have been cloned and sequenced (www.ncbi.nlm.nih.gov; www.humanabc.orq). Of these genes, 16 have known function and at least 14 have been associated with a defined human disease.

The functional ABC transporters typically contain two nucleotide-binding folds (NBF) and two transmembrane-spanning α-helices. ABC transporters bind to ATP and use the energy from the ATP hydrolysis to drive the transport of various molecules across cell membranes. These transporters are able to transport a variety of compounds across cell membranes against steep concentration gradients. The ABC transporters are involved in the transport of ions, amino acids, peptides, sugars, vitamins, steroid hormones, lipids, bile salts and toxic compounds across cell membranes.

The ABC transporters have been shown to be involved in transporting drugs out of cells, especially anti-cancer drugs. For example ABC B1 (MDR1), ABC C1 (MRP1), ABC C2 (MRP2), and ABC G2 (BCRP) have been characterized and tested for drug resistance. Genetic variations in the ABC transporters may modulate the phenotype in patients, and thus affect their predisposition to drug toxicity and response to drug treatment (Sparreboom et al., 2003).

The presence of functional ABC transporters in cells may significantly influence the efficacy of drugs. Thus, ABC transporter gene expression experiments in specific cells can be used to tailor drug treatment protocols to specific cell types, tissues, diseases or cancers. For example, a biopsy of a tumor can be tested for the presence of specific ABC transporter gene expression, and the information can be used to choose the most effective drugs for the treatment of that cancer. In addition, the information on ABC transporter gene expression can be used in candidate population profiling, such as the pre-screening of patients for inclusion or exclusion from clinical trials.

There is a need for screening of ABC transporter gene expression, which can be used, for example in drug screening analysis.

### SUMMARY OF THE INVENTION

The present inventors have prepared primers pairs for the human ABC transporter genes. These primers were used to generate a nucleic acid molecule for the ABC transporter genes, said nucleic acid molecule comprising a sequence that specifically hybridizes to only one of the ABC transporter genes. These nucleic acid molecules have been used in assays to screen for ABC transporter gene expression in test samples.

Accordingly, the present invention includes one or more isolated and purified nucleic acid molecules, wherein each of the nucleic acid molecules comprises a sequence that specifically hybridizes to one ABC transporter gene. In an embodiment of the invention the one or more nucleic acid molecules comprise a portion of the 3′ untranslated region of a human ABC transporter gene. In a further embodiment of the present invention, there is provided a set of at least two nucleic acid molecules, at least 10 nucleic acid molecules, at least 20 nucleic acid molecules, at least 30 nucleic acid molecules or at least 48 nucleic acid molecules, wherein each of the nucleic acid molecules comprises a sequence that specifically hybridizes to one ABC transporter gene. In another embodiment of the present invention, the set of at least two nucleic acid molecules are attached to a substrate. The substrate may be, for example, a membrane, a glass support, a filter, a tissue culture dish, a polymeric material, a bead or a silica support.

In an embodiment of the present invention, the one or more nucleic acid molecules comprise an isolated and purified nucleic acid sequence selected from those shown in FIGS. 1 to 47 and Sequence ID NOS: 1 to 47. In a further embodiment of the invention, the one or more nucleic acid molecules comprise an isolated and purified nucleic acid sequence selected from:
(a) the nucleic acid sequences as shown in SEQ ID NOS: 1 to 47 and FIGS. 1 to 47, wherein T can also be U;
(b) nucleic acid sequences complementary to (a);
(c) nucleic acid sequences which are homologous to (a) or (b); or
(d) a fragment of (a) to (c), which comprises a sequence that specifically hybridizes to one of the ABC transporter genes.

In an embodiment of the present invention the one or more nucleic acid molecules are prepared from one or more primer pairs using any known amplification method, for example the polymerase chain reaction (PCR). Accordingly, the present invention includes one or more pairs of primers for preparing one or more nucleic acid molecules, wherein each of the nucleic acid molecules comprises a sequence that specifically hybridizes to one ABC transporter gene. In an embodiment of the present invention, the one or more pairs of primers used to generate such nucleic acid molecules comprise a nucleic acid sequence selected from those listed in Table 1 or SEQ ID NOS: 48 to 141. In further embodiments of the invention, the primers comprise:
(a) the nucleic acid sequences as shown in SEQ ID NOS: 48 to 141 and Table 1, wherein T can also be U;
(b) nucleic acid sequences complementary to (a); or
(c) nucleic acid sequences which are homologous to (a) or (b).

In another embodiment of the invention, the primers comprise at least the 5 nucleotides at the 3′ end of the sequences as shown in Table 1 or SEQ ID NOS: 48 to 141.

In still further embodiments of the invention, the one or more primers pairs comprise a nucleic acid sequence selected from one or more of:
(a) SEQ ID NO: 48 and SEQ ID NO: 49;
   SEQ ID NO: 50 and SEQ ID NO: 51;
   SEQ ID NO: 52 and SEQ ID NO: 53;
   SEQ ID NO: 54 and SEQ ID NO: 55;
   SEQ ID NO: 56 and SEQ ID NO: 57;
   SEQ ID NO: 58 and SEQ ID NO: 59;
   SEQ ID NO: 60 and SEQ ID NO: 61;
   SEQ ID NO: 62 and SEQ ID NO: 63;
   SEQ ID NO: 64 and SEQ ID NO: 65;
   SEQ ID NO: 66 and SEQ ID NO: 67;
   SEQ ID NO: 68 and SEQ ID NO: 69;
   SEQ ID NO: 70 and SEQ ID NO: 71;
   SEQ ID NO: 72 and SEQ ID NO: 73;
   SEQ ID NO: 74 and SEQ ID NO: 75;
   SEQ ID NO: 76 and SEQ ID NO: 77;
   SEQ ID NO: 78 and SEQ ID NO: 79;
   SEQ ID NO: 80 and SEQ ID NO: 81;
   SEQ ID NO: 82 and SEQ ID NO: 83;
   SEQ ID NO: 84 and SEQ ID NO: 85;
   SEQ ID NO: 86 and SEQ ID NO: 87;
   SEQ ID NO: 88 and SEQ ID NO: 89;
   SEQ ID NO: 90 and SEQ ID NO: 91;
   SEQ ID NO: 92 and SEQ ID NO: 93;
   SEQ ID NO: 94 and SEQ ID NO: 95;
   SEQ ID NO: 96 and SEQ ID NO: 97;
   SEQ ID NO: 98 and SEQ ID NO: 99;
   SEQ ID NO: 100 and SEQ ID NO: 101;
   SEQ ID NO: 102 and SEQ ID NO: 103;
   SEQ ID NO: 104 and SEQ ID NO: 105;
   SEQ ID NO: 106 and SEQ ID NO: 107;
   SEQ ID NO: 108 and SEQ ID NO: 109;
   SEQ ID NO: 110 and SEQ ID NO: 111;
   SEQ ID NO: 112 and SEQ ID NO: 113;
   SEQ ID NO: 114 and SEQ ID NO: 115;
   SEQ ID NO: 116 and SEQ ID NO: 117;
   SEQ ID NO: 118 and SEQ ID NO: 119;
   SEQ ID NO: 120 and SEQ ID NO: 121;
   SEQ ID NO: 122 and SEQ ID NO: 123;
   SEQ ID NO: 124 and SEQ ID NO: 125;
   SEQ ID NO: 126 and SEQ ID NO: 127;
   SEQ ID NO: 128 and SEQ ID NO: 129;
   SEQ ID NO: 130 and SEQ ID NO: 131;
   SEQ ID NO: 132 and SEQ ID NO: 133;
   SEQ ID NO: 134 and SEQ ID NO: 135;
   SEQ ID NO: 136 and SEQ ID NO: 137;
   SEQ ID NO: 138 and SEQ ID NO: 139; and
   SEQ ID NO: 140 and SEQ ID NO: 141;
(b) the nucleic acid sequences in (a) wherein T can also be U;
(c) nucleic acid sequences complementary to (a) or (b); and
(d) nucleic acid sequences which are homologous to (a), (b) or (c).

The present invention also includes nucleic acid molecules prepared using PCR and one or more of the pairs of primers of the invention.

Additionally, the invention provides methods for detecting ABC transporter gene expression in general. Accordingly, the present invention includes a method of detecting the expression of one or more ABC transporter genes comprising:
(a) providing one or more nucleic acid molecules, each comprising a sequence that specifically hybridizes to one ABC transporter gene;
(b) providing a transcription indicator from a test sample;
(c) allowing the transcription indicator to hybridize with said one or more nucleic acid molecules; and
(d) detecting an amount of hybridization of said transcription indicator with said one or more nucleic acid sequences,
   wherein the amount of hybridization is indicative of the expression of one or more ABC transporter genes.

In another embodiment of the invention, an array, in particular a microarray is used to detect ABC transporter gene expression in a test sample. Therefore, the present invention also includes an array, in particular a microarray, comprising a substrate and one or more nucleic acid molecules, each comprising a sequence that specifically hybridizes to one ABC transporter gene, wherein said one or more nucleic acid molecules are immobilized to said substrate. Additionally, the invention provides a method of detecting ABC transporter gene expression in a test sample using a DNA microarray.

The nucleic acid molecules and methods of the present invention can be used to perform drug-associated ABC transporter gene expression profiling. Such profiling will identify potential modulators of ABC transporter gene expression. Accordingly, in yet another embodiment of the invention, there is provided a method for screening compounds for their effect on the expression of one or more ABC transporter genes comprising:
(a) exposing a test sample to one or more compounds;
(b) providing a transcription indicator from the test sample;
(c) providing one or more nucleic acid sequences, each comprising a sequence that specifically hybridizes to one ABC transporter gene;
(d) allowing said transcription indicator to hybridize with said one or more nucleic acid sequences; and
(e) detecting an amount of hybridization of said transcription indicator with said one or more nucleic acid sequences,
   wherein the amount of hybridization is indicative of the expression of the one or more ABC transporter genes.

In further embodiments, the methods of the invention further comprise (a) generating a set of expression data from the detection of the amount of hybridization; (b) storing the data in a database; and (c) performing comparative analysis on the set of expression data, thereby analyzing ABC transporter gene expression. The present invention also relates to a computer system comprising (a) a database containing information identifying the expression level of a set of genes comprising at least two ABC transporter genes; and (b) a user interface to view the information.

The method for screening compounds for their effect on ABC transporter gene expression is useful for the design of a drugs or chemical therapy for the treatment of disease. In an embodiment, the hybridization assay is a DNA microarray.

Other aspects of the present invention include kits for performing the methods of the invention as well as methods of conducting a target discovery business using the methods of the invention.

Other features and advantages of the present invention will become apparent from the following detailed description. It should be understood, however, that the detailed description and the specific examples while indicating embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 shows a nucleic acid sequence that specifically hybridizes to ABCA1 and corresponds to SEQ ID NO: 1.

FIG. 2 shows a nucleic acid sequence that specifically hybridizes to ABCA2 and corresponds to SEQ ID NO: 2.

FIG. 3 shows a nucleic acid sequence that specifically hybridizes to ABCA3 and corresponds to SEQ ID NO: 3.

FIG. 4 shows a nucleic acid sequence that specifically hybridizes to ABCA4 and corresponds to SEQ ID NO: 4.

FIG. 5 shows a nucleic acid sequence that specifically hybridizes to ABCA5 and corresponds to SEQ ID NO: 5.

FIG. 6 shows a nucleic acid sequence that specifically hybridizes to ABCA6 and corresponds to SEQ ID NO: 6.

FIG. 7 shows a nucleic acid sequence that specifically hybridizes to ABCA7 and corresponds to SEQ ID NO: 7.

FIG. 8 shows a nucleic acid sequence that specifically hybridizes to ABCA8 and corresponds to SEQ ID NO: 8.

FIG. 9 shows a nucleic acid sequence that specifically hybridizes to ABCA9 and corresponds to SEQ ID NO: 9.

FIG. 10 shows a nucleic acid sequence that specifically hybridizes to ABCA10 and corresponds to SEQ ID NO: 10.

FIG. 11 shows a nucleic acid sequence that specifically hybridizes to ABCA12 and corresponds to SEQ ID NO: 11.

FIG. 12 shows a nucleic acid sequence that specifically hybridizes to ABCB1 and corresponds to SEQ ID NO: 12.

FIG. 13 shows a nucleic acid sequence that specifically hybridizes to ABCB2 and corresponds to SEQ ID NO: 13.

FIG. 14 shows a nucleic acid sequence that specifically hybridizes to ABCB3 and corresponds to SEQ ID NO: 14.

FIG. 15 shows a nucleic acid sequence that specifically hybridizes to ABCB4 and corresponds to SEQ ID NO: 15.

FIG. 16 shows a nucleic acid sequence that specifically hybridizes to ABCB6 and corresponds to SEQ ID NO: 16.

FIG. 17 shows a nucleic acid sequence that specifically hybridizes to ABCB7 and corresponds to SEQ ID NO: 17.

FIG. 18 shows a nucleic acid sequence that specifically hybridizes to ABCB8 and corresponds to SEQ ID NO: 18.

FIG. 19 shows a nucleic acid sequence that specifically hybridizes to ABCB9 and corresponds to SEQ ID NO: 19.

FIG. 20 shows a nucleic acid sequence that specifically hybridizes to ABCB10 and corresponds to SEQ ID NO: 20.

FIG. 21 shows a nucleic acid sequence that specifically hybridizes to ABCB11 and corresponds to SEQ ID NO: 21.

FIG. 22 shows a nucleic acid sequence that specifically hybridizes to ABCC1 and corresponds to SEQ ID NO: 22.

FIG. 23 shows a nucleic acid sequence that specifically hybridizes to ABCC2 and corresponds to SEQ ID NO: 23.

FIG. 24 shows a nucleic acid sequence that specifically hybridizes to ABCC3 and corresponds to SEQ ID NO: 24.

FIG. 25 shows a nucleic acid sequence that specifically hybridizes to ABCC4 and corresponds to SEQ ID NO: 25.

FIG. 26 shows a nucleic acid sequence that specifically hybridizes to ABCC5 and corresponds to SEQ ID NO: 26.

FIG. 27 shows a nucleic acid sequence that specifically hybridizes to ABCC6 and corresponds to SEQ ID NO: 27.

FIG. 28 shows a nucleic acid sequence that specifically hybridizes to ABCC7 and corresponds to SEQ ID NO: 28.

FIG. 29 shows a nucleic acid sequence that specifically hybridizes to ABCC8 and corresponds to SEQ ID NO: 29.

FIG. 30 shows a nucleic acid sequence that specifically hybridizes to ABCC9 and corresponds to SEQ ID NO: 30.

FIG. 31 shows a nucleic acid sequence that specifically hybridizes to ABCC10b and corresponds to SEQ ID NO: 31.

FIG. 32 shows a nucleic acid sequence that specifically hybridizes to ABCC11 and corresponds to SEQ ID NO: 32.

FIG. 33 shows a nucleic acid sequence that specifically hybridizes to ABCC12a and corresponds to SEQ ID NO: 33.

FIG. 34 shows a nucleic acid sequence that specifically hybridizes to ABCC13 and corresponds to SEQ ID NO: 34.

FIG. 35 shows a nucleic acid sequence that specifically hybridizes to ABCD1 and corresponds to SEQ ID NO: 35.

FIG. 36 shows a nucleic acid sequence that specifically hybridizes to ABCD2 and corresponds to SEQ ID NO: 36.

FIG. 37 shows a nucleic acid sequence that specifically hybridizes to ABCD3 and corresponds to SEQ ID NO: 37.

FIG. 38 shows a nucleic acid sequence that specifically hybridizes to ABCD4 and corresponds to SEQ ID NO: 38.

FIG. 39 shows a nucleic acid sequence that specifically hybridizes to ABCE1 and corresponds to SEQ ID NO: 39.

FIG. 40 shows a nucleic acid sequence that specifically hybridizes to ABCF1 and corresponds to SEQ ID NO: 40.

FIG. 41 shows a nucleic acid sequence that specifically hybridizes to ABCF2 and corresponds to SEQ ID NO: 41.

FIG. 42 shows a nucleic acid sequence that specifically hybridizes to ABCF3 and corresponds to SEQ ID NO: 42.

FIG. 43 shows a nucleic acid sequence that specifically hybridizes to ABCG1 and corresponds to SEQ ID NO: 43.

FIG. 44 shows a nucleic acid sequence that specifically hybridizes to ABCG2 and corresponds to SEQ ID NO: 44.

FIG. 45 shows a nucleic acid sequence that specifically hybridizes to ABCG4 and corresponds to SEQ ID NO: 45.

FIG. 46 shows a nucleic acid sequence that specifically hybridizes to ABCG5 and corresponds to SEQ ID NO: 46.

FIG. 47 shows a nucleic acid sequence that specifically hybridizes to ABCG8 and corresponds to SEQ ID NO: 47.

FIG. 48 shows the ABC transporter gene RT-PCR amplification products from the CaCo2 cell line.

FIG. 49 shows the ABC transporter gene RT-PCR amplification products from the HEK293 cell line.

FIG. 50 shows the ABC transporter gene RT-PCR amplification products from the HepG2 cell line.

FIG. 51 shows a fluorescent intensity cluster plot of relative levels of ABC transporter gene expression in various cell lines normalized to GAPDH.

FIG. 52 shows a fluorescent intensity cluster plot of relative levels of ABC transporter gene expression in various cell lines normalized to actin.

FIG. 53 a fluorescent intensity cluster plot of relative levels of ABC transporter gene expression in various cell lines normalized to SH1.

FIG. 54 shows the relative levels of ABC B1 to B11 gene expression in the HEK cell line normalized to various constitutively expressed control genes.

FIG. 55 shows the relative levels of ABC B1 to B11 gene expression in various cell lines.

FIG. 56 shows a fluorescent intensity cluster plot of relative levels of ABC transporter gene expression in a cell line treated with doxorubicin at various time intervals.

FIG. 57 shows a fluorescent intensity cluster plot of relative levels of ABC transporter gene expression in a cell line treated with vinblastine at various time intervals.

FIG. 58 shows a matrix plot of the relative levels of ABC transporter gene expression in a cell line [HepG2] treated with either doxorubicin [dox] or vinblastine [vin] at various time intervals.

FIG. 59 shows a matrix plot of the relative levels of ABC transporter gene expression in several cell lines [A549, CaCo2, HepG2] treated with either acetaminophen [AP] or acetylsalicylic acid [SA].

FIG. 60 shows a matrix plot of the relative levels of ABC transporter gene expression in a cell line [A549] treated with either all-trans retinoic acid [AAT], cis-13 retinoic acid [A13], cis-9 retinoic acid [A9] or phorbol-12-myristate-13-acetate [APM].

FIG. 61 shows a matrix plot of the relative levels of ABC transporter gene expression in cell lines HTB81 [A], CRL1740 [C] and CRL2505 [D] treated with either no drug [none], methanol [Me], phenobarbitol [PhB], acetylsalicylic acid [ASA] or acetaminophen [AAP].

### DETAILED DESCRIPTION OF THE INVENTION

The present invention provides materials and methods for detection of ABC transporter gene expression. In particular, the invention relates to nucleic acid molecules for analyzing ABC transporter gene expression, wherein the nucleic acid molecules comprise a sequence that specifically hybridizes to one ABC transporter gene, and methods and materials for obtaining such nucleic acid molecules. The invention also relates to the use of said materials and methods in assays and kits to detect ABC transporter gene expression.

(I) Abbreviations

The following standard abbreviations for the nucleic acid residues are used throughout the specification: A-adenine; C-cytosine; G-guanine; T-thymine; and U-uracil.

(II) Definitions

The term “nucleic acid molecule”, “nucleic acid sequence(s)” or “nucleotide sequence” as used herein refers to an oligonucleotide or polynucleotide, and fragments or portions thereof, and to DNA or RNA of genomic or synthetic origin that may be single- or double-stranded, and represent the sense or antisense strand.

The term “ABC transporter genes” refers to nucleic acid sequences encoding the ABC transporters, for example the human ABC transporter genes. There are currently 48 known human transporters, which have been cloned and sequenced (www.ncbi.nlm.nih.gov; www.humanabc.org). The discovery and confirmation of new ABC transporter genes are ongoing. ABC transporter genes in this application are intended to include unknown ABC transporter genes, which will be discovered or confirmed in the future.

The term “PCR amplicon” refers to a nucleic acid generated by nucleic acid amplification.

The term “ABC transporter gene expression” refers to the transcription of an ABC transporter gene into an RNA product.

“Amplification” is defined as the production of additional copies of a nucleic acid sequence and is generally carried out using polymerase chain reaction technologies well known in the art (Dieffenbach C W and G S Dveksler (1995) PCR Primer, a Laboratory Manual, Cold Spring Harbor Press, Plainview N.Y.). As used herein, the term “polymerase chain reaction” (“PCR”) refers to the method of K. B. Mullis U.S. Pat. Nos. 4,683,195 and 4,683,202, hereby incorporated by reference, which describe a method for increasing the concentration of a segment of a target sequence in a mixture of genomic DNA without cloning or purification. The length of the amplified segment of the desired target sequence is determined by the relative positions of two oligonucleotide primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

Amplification in PCR requires “PCR reagents” or “PCR materials”, which herein are defined as all reagents necessary to carry out amplification except the polymerase, primers and template. PCR reagents normally include nucleic acid precursors (dCTP, dTTP etc.) and buffer.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer can be single stranded for maximum efficiency in amplification, but may alternatively be double stranded. If double stranded, the primer is first treated to separate its strands before being used to prepare extension products. In one embodiment, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

The term “pair(s) of primers” refers to an upper primer and a lower primer. The primers can be categorized as upper or lower primers, depending upon the relative orientation of the primer versus the polarity of the nucleic acid sequence of interest (e.g., whether the primer binds to the coding strand or a complementary (noncoding) strand of the sequence of interest).

The terms “homolog” “homology” and “homologous” as used herein in reference to nucleotides or nucleic acid sequences refer to a degree of complementarity with other nucleotides or nucleic acid sequences. There may be partial homology or complete homology (i.e., identity). A nucleotide sequence that is partially complementary, i.e., “substantially homologous,” to a nucleic acid sequence is one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence that lacks even a partial degree of complementarity (e.g., less than about 30% identity); in the absence of non-specific binding the probe will not hybridize to the second non-complementary target.

Low stringency conditions comprise conditions equivalent to binding or hybridization at 25° C., in a solution consisting of 500 mM sodium phosphate pH 6.0, 1% SDS, 1% BSA, 1 mM EDTA when a target of about 50 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol), as well as components of the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

When used in reference to a single-stranded nucleic acid sequence, the term “substantially homologous” refers to any probe that can hybridize (i.e., it is the complement of the single-stranded nucleic acid sequence) under conditions of low stringency as described above.

The term “cDNA” refers to complementary or “copy” DNA. Generally, cDNA is synthesized by a DNA polymerase using any type of RNA molecule as a template. Alternatively, the cDNA can be obtained by direct chemical synthesis.

The term “complementary” refers to nucleic acid sequences capable of base-pairing according to the standard Watson-Crick complementary rules, or being capable of hybridizing to a particular nucleic acid segment under stringent conditions.

The term “hybridization” refers to duplex formation between two or more polynucleotides to form, for example a double-stranded nucleic acid, via base pairing. The ability of two regions of complementarity to hybridize and remain together depends on the length and continuity of the complementary regions, and the stringency of the hybridization conditions.

The term “DNA microarray” refers to substrate with at least one target DNA immobilized to said substrate. The target DNA molecules are typically immobilized in prearranged patterns so that their locations are known or determinable. Nucleic acids in a sample can be detected by contacting the sample with the DNA microarray; allowing the target DNA and nucleic acids in the sample to hybridize; and analyzing the extent of hybridization.

The term “label” refers to any detectable moiety. A label may be used to distinguished a particular nucleic acid from others that are unlabelled, or labeled differently, or the label may be used to enhance detection.

The term “nucleic acids” refers to a polymer of ribonucleic acids or deoxyribonucleic acids, including RNA, mRNA, rRNA, tRNA, small nuclear RNAs, cDNA, DNA, PNA, or RNA/DNA copolymers. Nucleic acid may be obtained from a cellular extract, genomic or extragenomic DNA, viral RNA or DNA, or artificially/chemically synthesized molecules.

The term “RNA” refers to a polymer of ribonucleic acids, including RNA, mRNA, rRNA, tRNA and small nuclear RNAS, as well as to RNAs that comprise ribonucleotide analogues to natural ribonucleic acid residues, such as 2-O-methylated residues.

The term “transcription” refers to the process of copying a DNA sequence of a gene into an RNA product, generally conducted by a DNA-directed RNA polymerase using the DNA as a template.

The term “isolated” when used in relation to a nucleic acid molecule or sequence, refers to a nucleic acid sequence that is identified and separated from at least one contaminant nucleic acid with which it is ordinarily associated in its natural source. Isolated nucleic acid is nucleic acid present in a form or setting that is different from that in which it is found in nature.

As used herein, the term “purified” or “to purify” refers to the removal of undesired components from a sample.

As used herein, the term “substantially purified” refers to molecules, either nucleic or amino acid sequences, that are removed from their natural environment, isolated or separated, and are at least 60% free, 75% free, or 90% free from other components with which they are naturally associated. An “isolated nucleic acid molecule” is therefore a substantially purified nucleic acid molecule.

(Ill) Nucleic Acid Molecules

The present invention provides one or more isolated and purified nucleic acid molecules, wherein each of the nucleic acid molecules comprises a sequence that specifically hybridizes to only one ABC transporter gene. By “specifically hybridizes to” it is meant that the subject nucleic acid sequence will bind, duplex or hybridize substantially to or only with a particular nucleic acid sequence with minimum cross-hybidization with the other members of this gene family. In other words, the nucleic acid sequence represents a probe for one ABC transporter gene. In an embodiment of the invention, the one or more nucleic acid molecules comprise a portion of the 3′ untranslated region of a human ABC transporter gene.

In a further embodiment of the present invention, there is provided a set of at least two nucleic acid molecules, at least 10 nucleic acid molecules, at least 20 nucleic acid molecules, at least 30 nucleic acid molecules or at least 48 nucleic acid molecules, wherein each of the nucleic acid molecules comprises a sequence that specifically hybridizes to one ABC transporter gene. In another embodiment of the present invention, the set of at least two nucleic acid molecules are attached to a substrate. The substrate may be, for example, a membrane, a glass support, a filter, a tissue culture dish, a polymeric material, a bead or a silica support.

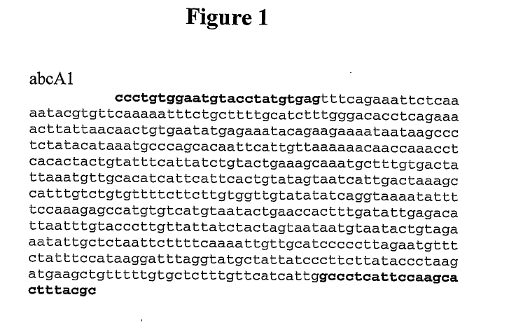

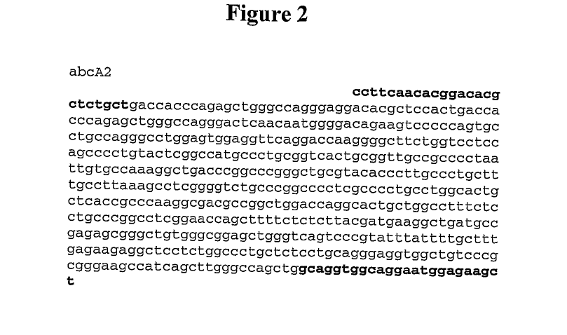

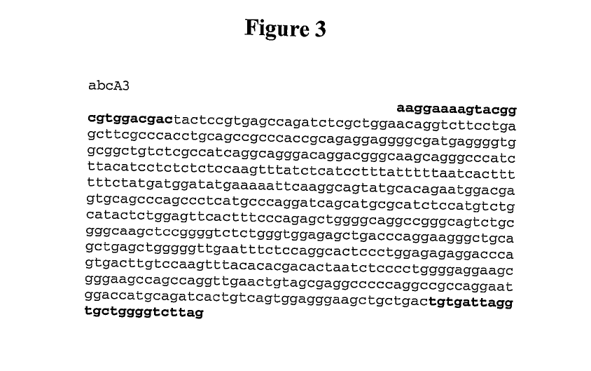

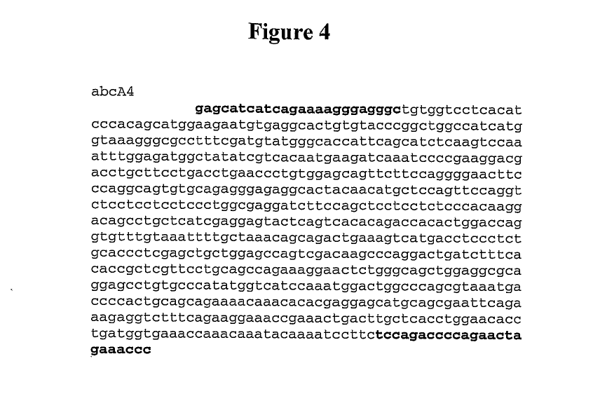

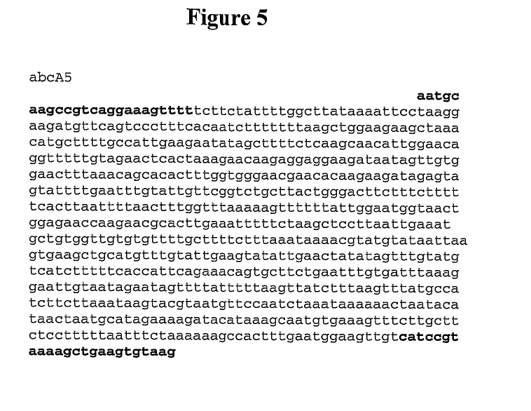

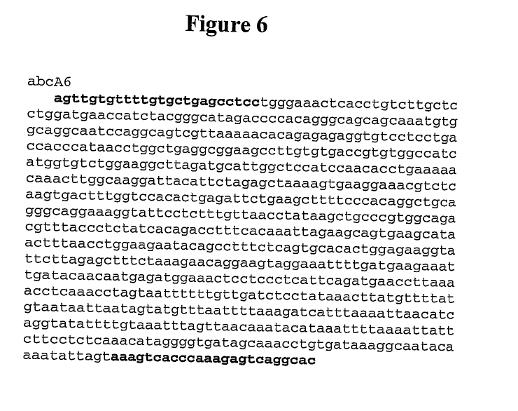

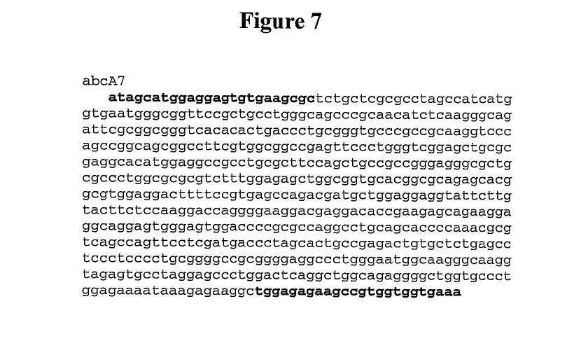

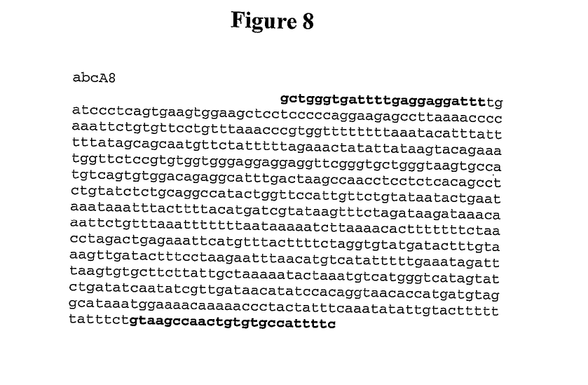

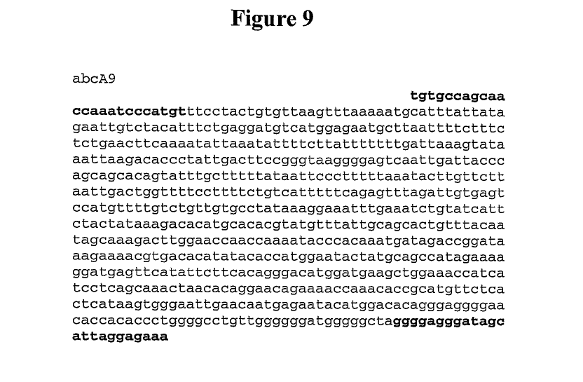

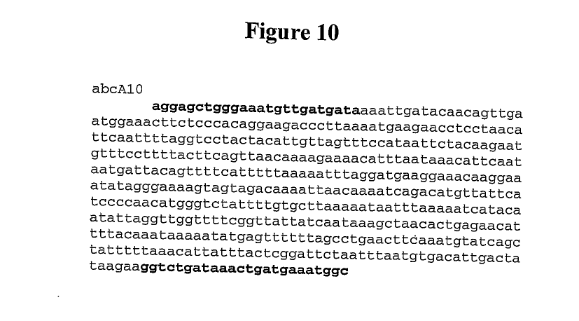

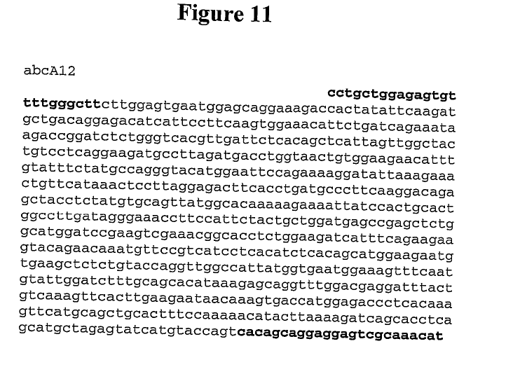

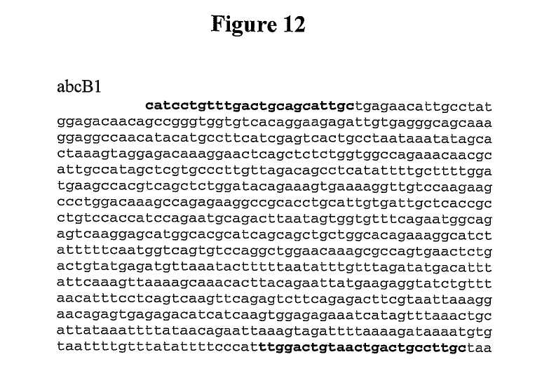

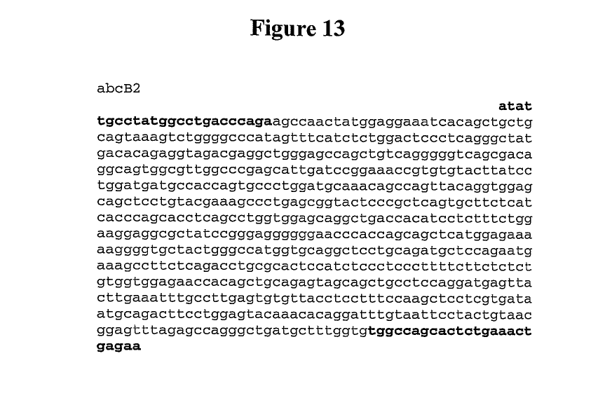

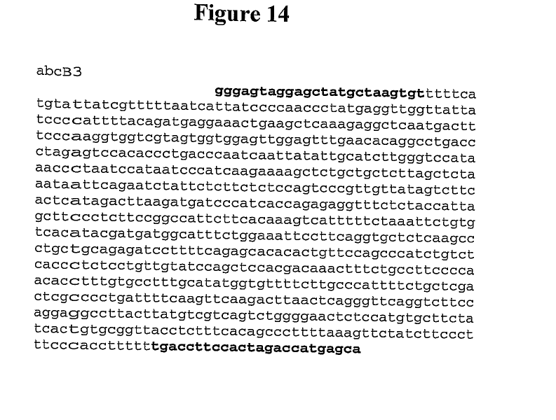

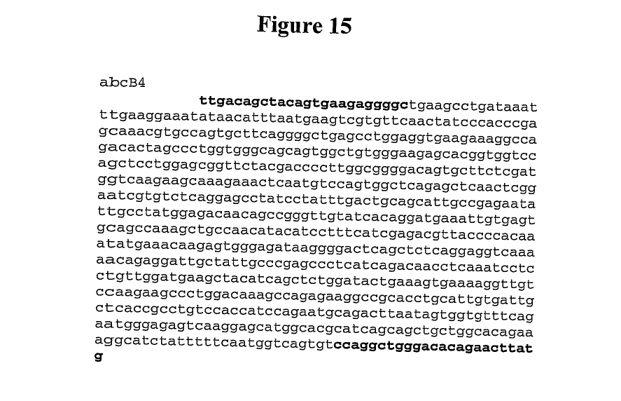

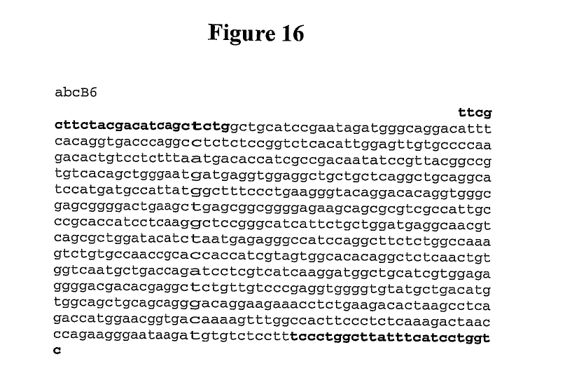

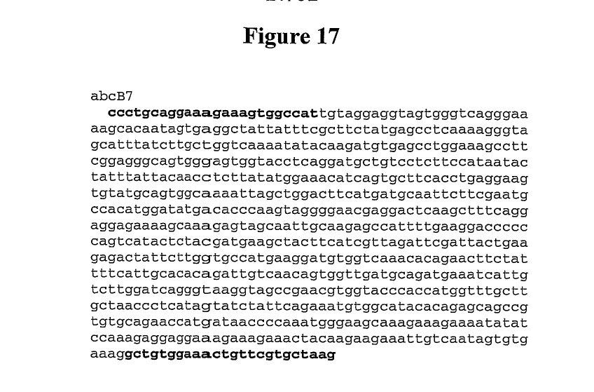

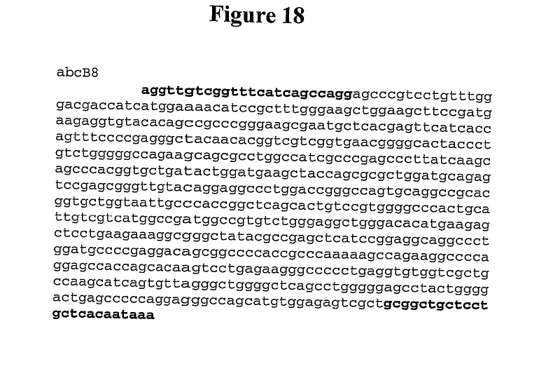

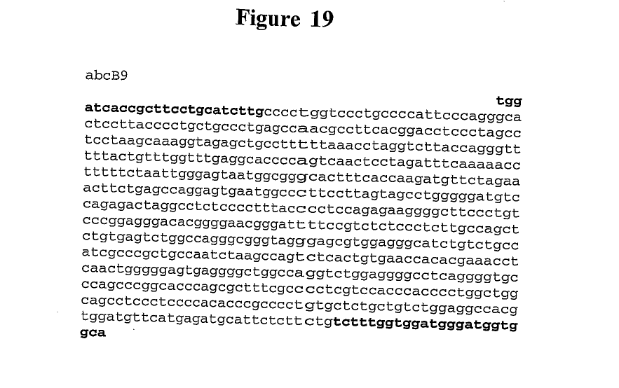

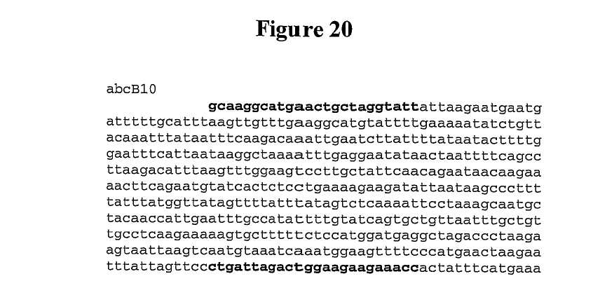

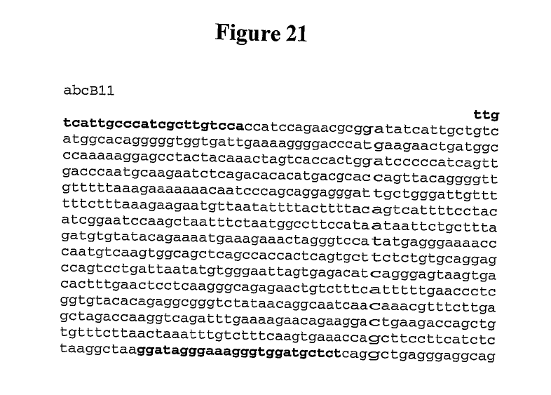

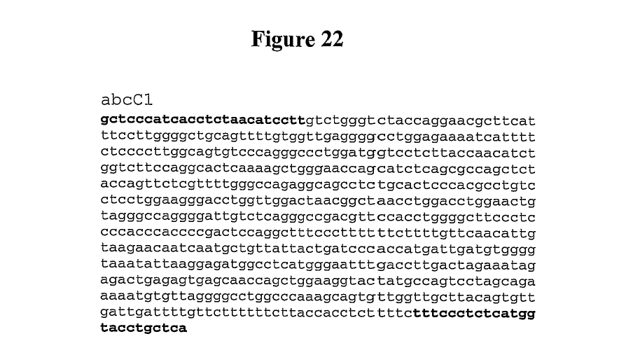

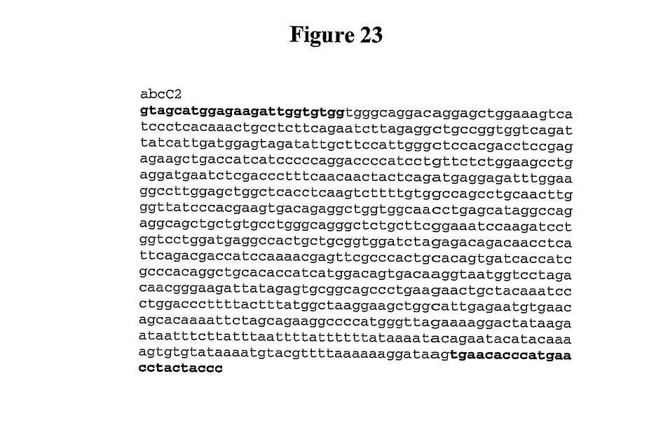

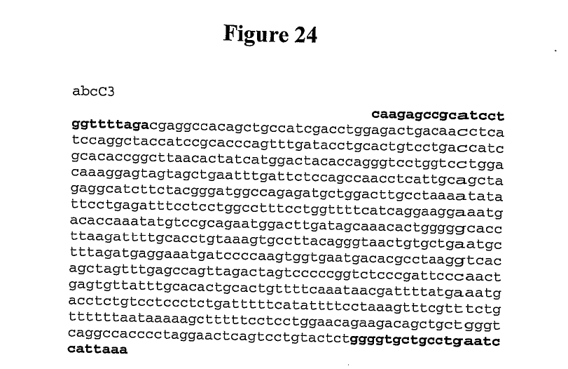

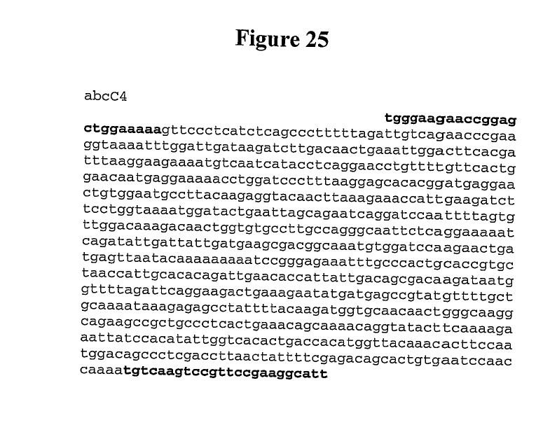

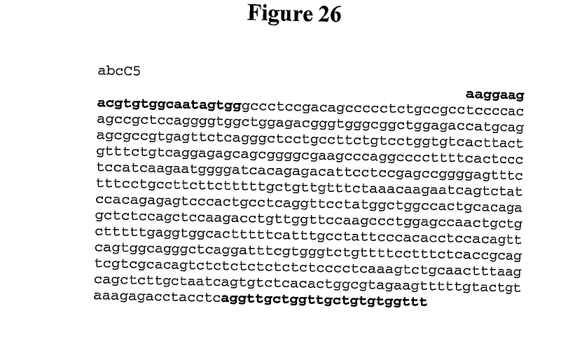

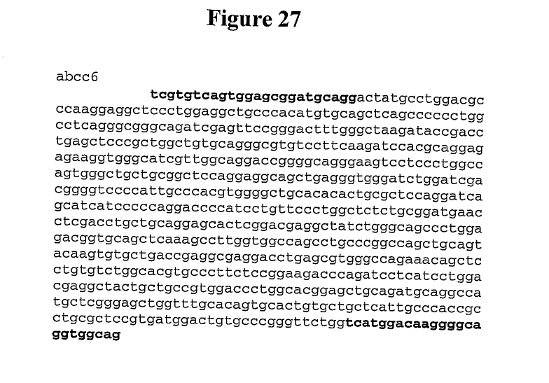

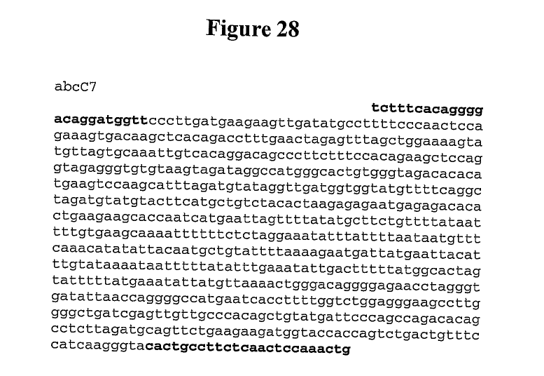

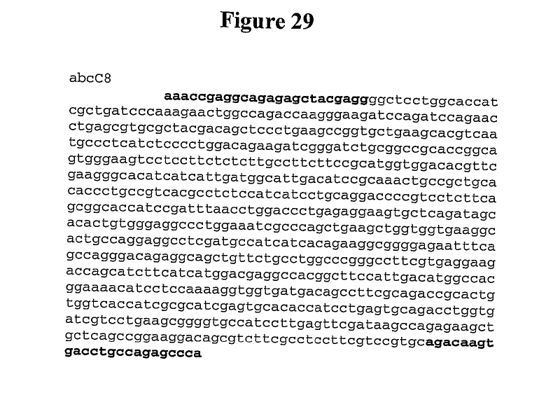

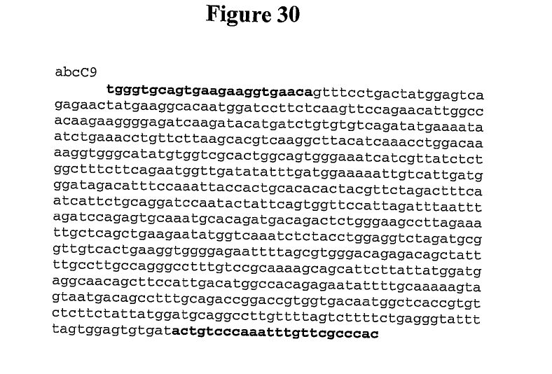

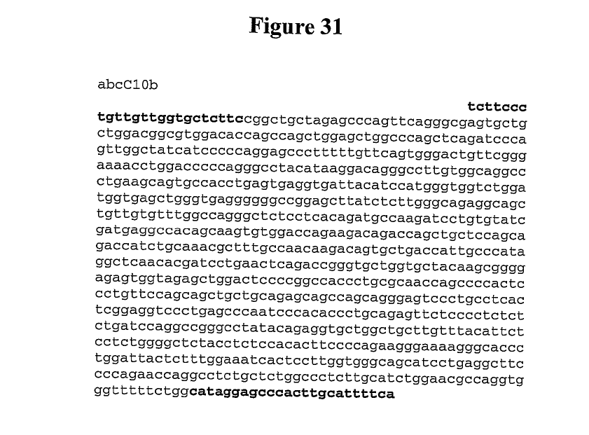

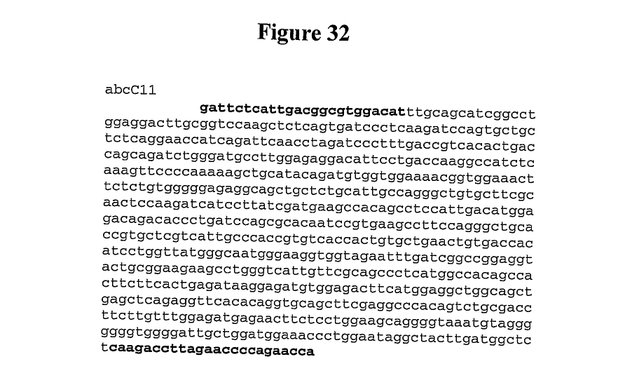

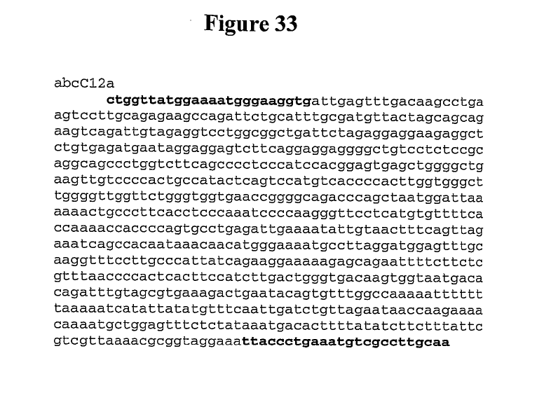

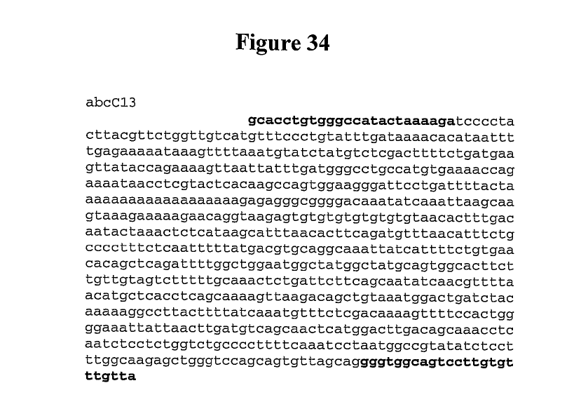

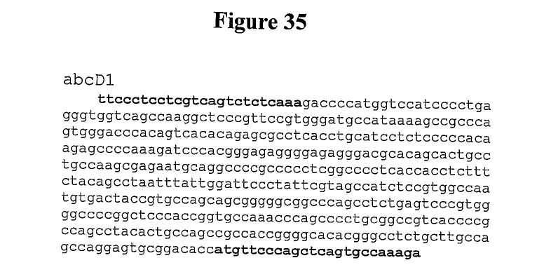

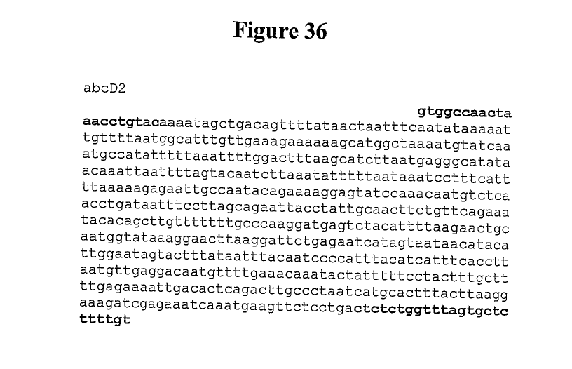

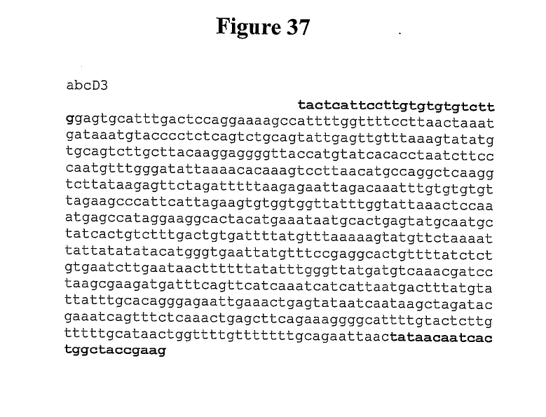

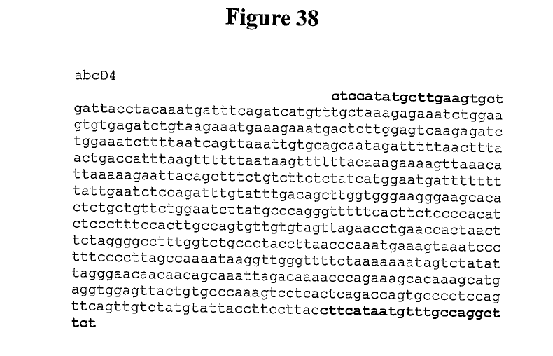

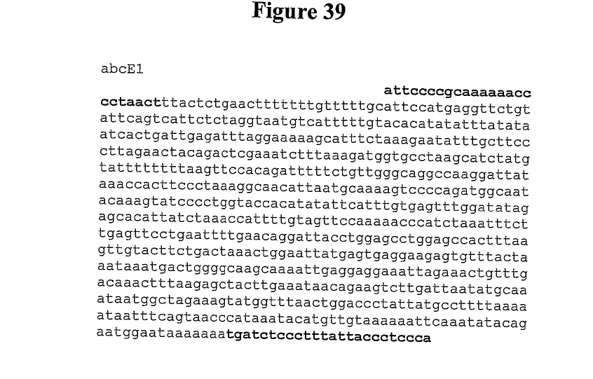

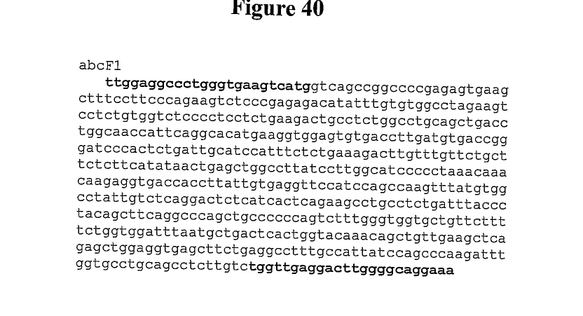

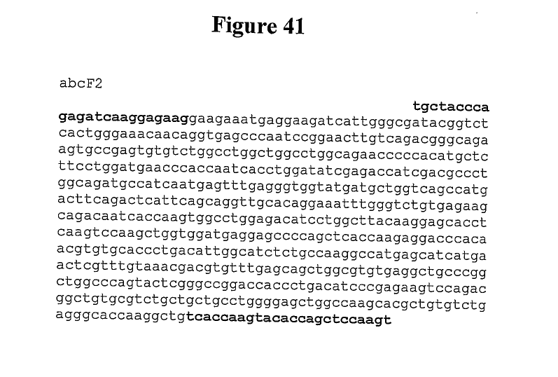

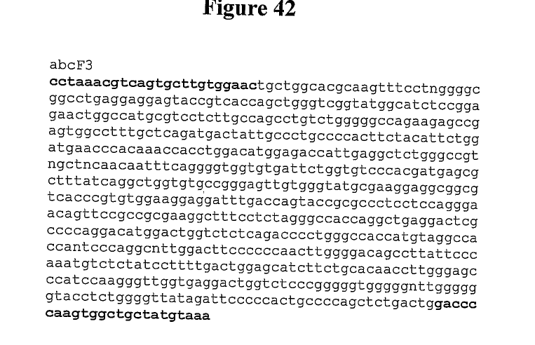

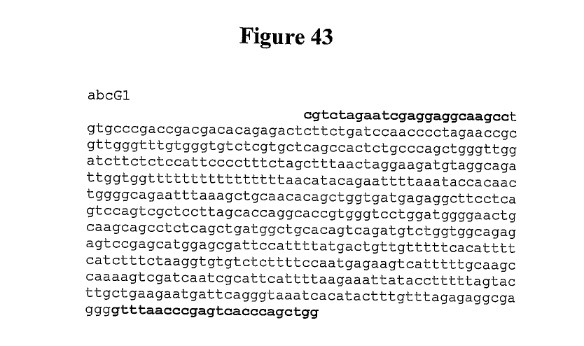

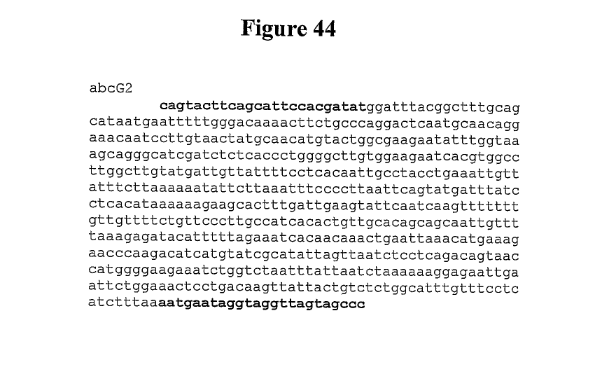

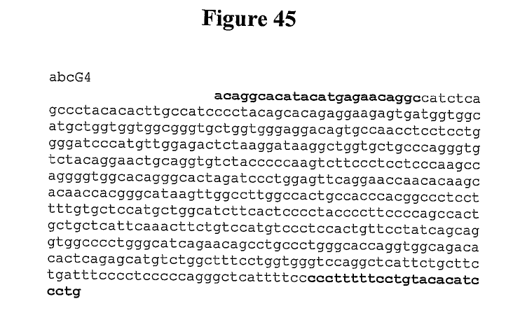

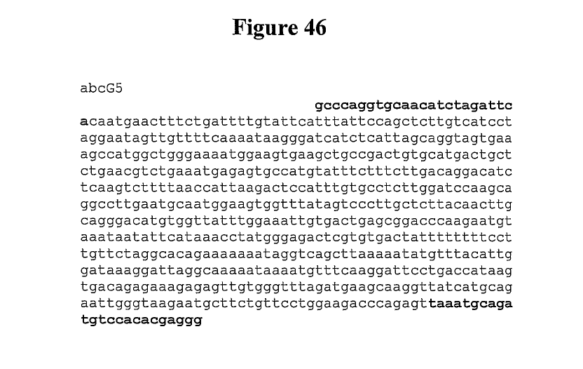

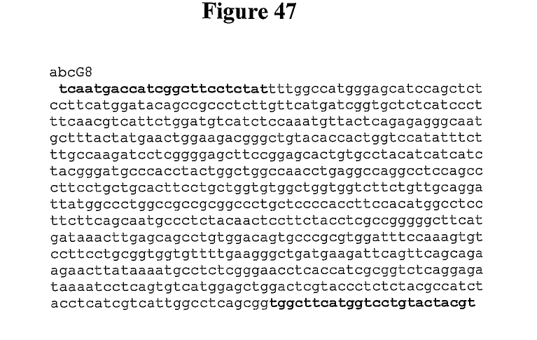

In an embodiment of the present invention, the one or more nucleic acid molecules comprise an isolated and purified nucleic acid sequence selected from those shown in FIGS. 1 to 47 and Sequence ID NOS: 1 to 47. In a further embodiment of the invention, the one or more nucleic acid molecules comprise an isolated and purified nucleic acid sequence selected from:
(a) the nucleic acid sequences as shown in SEQ ID NOS: 1 to 47 and FIGS. 1 to 47, wherein T can also be U;
(b) nucleic acid sequences complementary to (a);
(c) nucleic acid sequences which are homologous to (a) or (b); or
(d) a fragment of (a) to (c), which comprises a sequence that specifically hybridizes to one of the ABC transporter genes.

In an embodiment of the present invention the one or more nucleic acid molecules are prepared from one or more primer pairs using any known amplification method, for example the polymerase chain reaction (PCR). Accordingly, the present invention includes one or more pairs of primers for preparing one or more nucleic acid molecules, wherein each of the nucleic acid molecules comprises a sequence that specifically hybridizes to one ABC transporter gene. In an embodiment of the present invention, the one or more pairs of primers used to generate such nucleic acid molecules comprise a nucleic acid sequence selected from those listed in Table 1 or SEQ ID NOS: 49 to 144. In further embodiments of the invention, the primers comprise:
(a) the nucleic acid sequences as shown in SEQ ID NOS: 48 to 141 and Table 1, wherein T can also be U;
(b) nucleic acid sequences complementary to (a); or
(c) nucleic acid sequences which are homologous to (a) or (b).

In another embodiment of the invention, the primers comprise at least the 5 nucleotides at the 3′ end of the sequences as shown in Table 1 or SEQ ID NOS: 48 to 141.

In still further embodiments of the invention, the one or more primers pairs comprise a nucleic acid sequence selected from one or more of:
(a) one or more isolated and purified pairs of nucleic acid sequences selected from:
   SEQ ID NO: 48 and SEQ ID NO: 49;
   SEQ ID NO: 50 and SEQ ID NO: 51;
   SEQ ID NO: 52 and SEQ ID NO: 53;
   SEQ ID NO: 54 and SEQ ID NO: 55;
   SEQ ID NO: 56 and SEQ ID NO: 57;
   SEQ ID NO: 58 and SEQ ID NO: 59;
   SEQ ID NO: 60 and SEQ ID NO: 61;
   SEQ ID NO: 62 and SEQ ID NO: 63;
   SEQ ID NO: 64 and SEQ ID NO: 65;
   SEQ ID NO: 66 and SEQ ID NO: 67;
   SEQ ID NO: 68 and SEQ ID NO: 69;
   SEQ ID NO: 70 and SEQ ID NO: 71;
   SEQ ID NO: 72 and SEQ ID NO: 73;
   SEQ ID NO: 74 and SEQ ID NO: 75;
   SEQ ID NO: 76 and SEQ ID NO: 77;
   SEQ ID NO: 78 and SEQ ID NO: 79;
   SEQ ID NO: 80 and SEQ ID NO: 81;
   SEQ ID NO: 82 and SEQ ID NO: 83;
   SEQ ID NO: 84 and SEQ ID NO: 85;
   SEQ ID NO: 86 and SEQ ID NO: 87;
   SEQ ID NO: 88 and SEQ ID NO: 89;
   SEQ ID NO: 90 and SEQ ID NO: 91;
   SEQ ID NO: 92 and SEQ ID NO: 93;
   SEQ ID NO: 94 and SEQ ID NO: 95;
   SEQ ID NO: 96 and SEQ ID NO: 97;
   SEQ ID NO: 98 and SEQ ID NO: 99;
   SEQ ID NO: 100 and SEQ ID NO: 101;
   SEQ ID NO: 102 and SEQ ID NO: 103;
   SEQ ID NO: 104 and SEQ ID NO: 105;
   SEQ ID NO: 106 and SEQ ID NO: 107;
   SEQ ID NO: 108 and SEQ ID NO: 109;
   SEQ ID NO: 110 and SEQ ID NO: 111;
   SEQ ID NO: 112 and SEQ ID NO: 113;
   SEQ ID NO: 114 and SEQ ID NO: 115;
   SEQ ID NO: 116 and SEQ ID NO: 117;
   SEQ ID NO: 118 and SEQ ID NO: 119;
   SEQ ID NO: 120 and SEQ ID NO: 121;
   SEQ ID NO: 122 and SEQ ID NO: 123;
   SEQ ID NO: 124 and SEQ ID NO: 125;
   SEQ ID NO: 126 and SEQ ID NO: 127;
   SEQ ID NO: 128 and SEQ ID NO: 129;
   SEQ ID NO: 130 and SEQ ID NO: 131;
   SEQ ID NO: 132 and SEQ ID NO: 133;
   SEQ ID NO: 134 and SEQ ID NO: 135;
   SEQ ID NO: 136 and SEQ ID NO: 137;
   SEQ ID NO: 138 and SEQ ID NO: 139; and
   SEQ ID NO: 140 and SEQ ID NO: 141;
(b) the nucleic acid sequences in (a) wherein T can also be U;
(c) nucleic acid sequences complementary to (a) or (b); and
(d) nucleic acid sequences which are homologous to (a), (b) or (c).

The present invention also includes nucleic acid molecules prepared using PCR and one or more of the pairs of primers of the invention.

(IV) Method for Detecting ABC Transporter Gene Expression

Transcription of genes into RNA is a critical step in gene expression. Therefore gene expression can be monitored by monitoring various transcription indicators. There are a variety of techniques known in the art to analyze and quantify gene transcription. In an embodiment of the present invention, ABC transporter gene expression was detected by monitoring or detecting the hybridization of transcription indicators from a test sample with the one or more nucleic acid molecules of the present invention, wherein the one or more nucleic acid molecules comprise a sequence that specifically hybridizes to one ABC transporter gene. In an embodiment, ABC transporter gene expression was detected using reverse transcription. For example, RNA was extracted from a test sample using techniques known in the art. cDNA was then synthesized using known techniques, such as using either oligo(dT) or random primers. ABC transporter gene expression was then detected using the said cDNA by allowing the cDNA to hybridize to the one or more nucleic acid molecules, then detecting the amount of hybridization of said cDNA with the one or more nucleic acid molecules.

Accordingly, the present invention includes a method of detecting the expression of one or more ABC transporter genes comprising:
(a) providing one or more nucleic acid molecules, each comprising a sequence that specifically hybridizes to one ABC transporter gene;
(a) providing transcription indicators from a test sample;
(b) allowing the transcription indicators to hybridize with said one or more nucleic acid molecules; and
(c) detecting an amount of hybridization of said transcription indicators with said one or more nucleic acid sequences,
   wherein the amount of hybridization is indicative of the expression of one or more ABC transporter genes.
   (a) Transcription Indicators

One of skill in the art will appreciate that it is desirable to have transcription indicators from a test sample that contain suitable nucleic samples having target nucleic acid sequences that reflect the transcripts of interest. Therefore, suitable nucleic acid samples from the test sample may contain transcripts of interest. Suitable nucleic acid samples, however, may contain nucleic acids derived from the transcripts of interest. As used herein, a nucleic acid derived from a transcript refers to a nucleic acid for whose synthesis the mRNA transcript or a subsequence thereof has ultimately served as a template. Thus, a cDNA reverse transcribed from a transcript, an RNA transcribed from that cDNA, a DNA amplified from the cDNA, an RNA transcribed from the amplified DNA, etc., are all derived from the transcript and detection of such derived products is indicative of the presence and/or abundance of the original transcript in a sample. Thus, suitable transcription indicators include, but are not limited to, transcripts of the gene or genes, cDNA reverse transcribed from the transcript, cRNA transcribed from the cDNA, DNA amplified from the genes, RNA transcribed from amplified DNA, and the like. In an embodiment the transcription indicator is cDNA.

Transcripts, as used herein, may include, but not limited to pre-mRNA nascent transcript(s), transcript processing intermediates, mature mRNA(s) and degradation products. It is not necessary to monitor all types of transcripts to practice this invention. For example, one may choose to practice the invention to measure the mature mRNA levels only.

The term “test sample” refers to one or more cells, cell lines, tissues or organisms, or fragments thereof. In one embodiment, the test sample is from a human. In an embodiment of the present invention, the test sample is a homogenate of cells or tissues or other biological samples. For example, such sample can be a total RNA preparation of a biological sample or such a nucleic acid sample can be the total mRNA isolated from a biological sample. Those of skill in the art will appreciate that the total mRNA prepared with most methods includes not only the mature mRNA, but also the RNA processing intermediates and nascent pre-mRNA transcripts. For example, total mRNA purified with a poly (dT) column contains RNA molecules with poly (A) tails. Those polyA+RNA molecules could be mature mRNA, RNA processing intermediates, nascent transcripts or degradation intermediates.

In an embodiment of the present invention, the test sample is a clinical sample with is a sample derived from a patient. Typical clinical samples include, but are not limited to, sputum, blood, blood cells (e.g. white blood cells), tissue or fine needle biopsy samples, urine, peritoneal fluid and pleural fluid, or cells therefrom. In another embodiment of the present invention, the test sample is derived from a cell culture containing specific cell lines, for example, HepG2, CaCo2 or HEK 293.

One skilled in the art will appreciate that one can inhibit or destroy RNase present in any sample before they are used in the methods of the invention. Methods of inhibiting or destroying nucleases, including RNase, are well known in the art. For example, chaotropic agents may be used to inhibit nucleases or, alternatively, heat treatment followed by proteinase treatment may be used.

Methods of isolating total mRNA are also well known to those skilled in the art. For example, see Chapter 3 of Laboratory Techniques in Biochemistry and Molecular Biology: Hybridization with Nucleic Acid Probes, Part I: Theory and Nucleic Acid Preparation, Tijssen, ed. Elsevier Press (1993); Sambrook et al., Molecular Cloning: A Laboratory Manual (2^{nd} ed.), Vols. 1-3, Cold Spring Harbour Laboratory (1989); or Current Protocols in Molecular Biology, F. Ausubel et al., ed. Greene Publishing and Wiley-Interscience, New York (1987). In an embodiment, the total RNA is isolated from a given test sample, for example, using TRIzol reagent (Cat. No. 15596-018, Invitrogen Life Technologies) according to the manufacturer's instructions.

In embodiments of the present invention, the transcription indicator, whether it be cDNA or mRNA, may need to be amplified prior to performing the hybridization assay. Methods for amplification, including “quantitative amplification” are well known to those skilled in the art.

In an embodiment the transcription indicator is labeled with a detectable label. Methods for labeling nucleic acids are well known to those skilled in the art. In an embodiment of the invention, the label is simultaneously incorporated during an amplification step in the preparation of the transcription indicators. Thus for example, PCR with labeled primers or labeled nucleotides (for example fluorescein-labeled UTP and/or CTP) will provide a labeled amplification product. Alternatively, a label may be added directly to the original nucleic acid sample or to the amplification product after the amplification is completed using methods known to those skilled in the art (for example nick translation and end-labeling).

Detectable labels that are suitable for use in the methods of the present invention, include those that are detectable by spectroscopic, photochemical, biochemical, immunochemical, electrical, optical or other means. Some examples of useful labels include biotin staining with labeled streptavidin conjugate, magnetic beads, fluorescent dyes (e.g. fluorescein, rhodamine, green fluorescent protein and the like), radiolabels (e.g. ³H, ³²P, ¹⁴C, ²⁵S or ¹²⁵I), enzymes (e.g. horseradish peroxidase, alkaline phosphatase and others commonly used in ELISA) and calorimetric labels such as colloidal gold or colored glass or plastic (e.g. polystyrene, polypropylene, latex and the like) beads. Patents teaching the use of such labels include U.S. Pat. Nos. 3,817,837, 3,850,752, 3,939,350, 3,996,345, 4,277,437, 4,275,149 and 4,366,241, the contents of all of which are incorporated herein by reference.

(b) Assay Format

The method of detecting ABC transporter gene expression can be performed using any hybridization assay, including solution and solid phase. Typically a set containing two or more nucleic acid molecules of the invention, each of said nucleic acid molecules comprising a sequence that specifically hybridizes to one ABC transporter gene, are put together in a common container or on a common object. These may be on an array or in a kit together. They are typically separated, either spatially on a solid support such as an array, or in separate vessels, such as vials, tubes or wells in a microwell plate.

According to the present invention, at least 5% of the nucleic acid molecules or probes in a set comprise a sequence that specifically hybridizes to one ABC transporter gene. In an embodiment, more than 10%, 20%, 30%, 40%, 50%, 60%, 70%, 80%, 90%, or 95% of such nucleic acid molecules or probes in the set comprise a sequence that specifically hybridizes to one ABC transporter gene.

In an embodiment of the present invention the method of detecting ABC transported gene expression is performed in an array format. One of skill in the art will appreciate that an enormous number of array designs are suitable for the practice of this invention. The array will typically include a number of nucleic acid molecules or probes that specifically hybridize to the sequences of interest. In addition, in an embodiment, the array will include one or more control nucleic acid molecules or probes. The control probes may be, for example, expression level controls (e.g. positive controls and background negative controls).

Background controls are elements printed on the substrate that contain no nucleic acids and thus measure the amount of non-specific hybridization of the labelled cDNA to elements on the substrate.

Expression level controls are probes that hybridize specifically with constitutively expressed genes in the biological sample. Virtually any constitutively expressed gene provides a suitable target for expression level controls. Typically expression level control probes have sequences complementary to subsequences of constitutively expressed “housekeeping genes” including, but not limited to the beta-actin gene, the transferrin receptor gene, the glyceraldehyde-3-phosphate dehydrogenase (GAPDH) gene, and the like [Warrington J A et al., Physiol Genomics 2:143-147, 2000, Hsiao L L et al., Physiol Genomics 7:97-104, 2001, Whiffield M L et al., Mol Cell Biol 13:1977-2000, 2002].

In embodiments of the invention the method of detecting ABC transporter expression in a test sample is performed once or more, over a set period of time and at specified intervals, to monitor ABC transporter expression over that period of time.

DNA microarrays have the benefit of assaying gene expression in a high throughput fashion. These microarrays comprise short nucleic acid sequences that are immobilized on or directly chemically synthesized on a substrate, which can then be used in a hybridization reaction with nucleotides extracted from a test sample. Microarrays have the advantage of being able to measure the expression level of hundreds of genes simultaneously.

Accordingly, in an embodiment of the present invention there is provided a DNA microarray comprising one or more nucleic acid molecules arrayed on a substrate, wherein each of the one or more nucleic acid molecules comprise a sequence that specifically hybridizes to one ABC transporter gene. In an embodiment of the invention, the one or more nucleic acid molecules are selected from:
(a) the nucleic acid sequences as shown in SEQ ID NOS: 1 to 47 and FIGS. 1 to 47, wherein T can also be U;
(b) nucleic acid sequences complementary to (a);
(c) nucleic acid sequences which are homologous to (a) or (b); and
(d) a fragment of (a) to (c), which comprises a sequence that specifically hybridizes to one of the ABC transporter genes, or
one or more nucleic acids prepared using PCR and one or more primer pairs selected from:
(a) SEQ ID NO: 48 and SEQ ID NO: 49;
   SEQ ID NO: 50 and SEQ ID NO: 51;
   SEQ ID NO: 52 and SEQ ID NO: 53;
   SEQ ID NO: 54 and SEQ ID NO: 55;
   SEQ ID NO: 56 and SEQ ID NO: 57;
   SEQ ID NO: 58 and SEQ ID NO: 59;
   SEQ ID NO: 60 and SEQ ID NO: 61;
   SEQ ID NO: 62 and SEQ ID NO: 63;
   SEQ ID NO: 64 and SEQ ID NO: 65;
   SEQ ID NO: 66 and SEQ ID NO: 67;
   SEQ ID NO: 68 and SEQ ID NO: 69;
   SEQ ID NO: 70 and SEQ ID NO: 71;
   SEQ ID NO: 72 and SEQ ID NO: 73;
   SEQ ID NO: 74 and SEQ ID NO: 75;
   SEQ ID NO: 76 and SEQ ID NO: 77;
   SEQ ID NO: 78 and SEQ ID NO: 79;
   SEQ ID NO: 80 and SEQ ID NO: 81;
   SEQ ID NO: 82 and SEQ ID NO: 83;
   SEQ ID NO: 84 and SEQ ID NO: 85;
   SEQ ID NO: 86 and SEQ ID NO: 87;
   SEQ ID NO: 88 and SEQ ID NO: 89;
   SEQ ID NO: 90 and SEQ ID NO: 91;
   SEQ ID NO: 92 and SEQ ID NO: 93;
   SEQ ID NO: 94 and SEQ ID NO: 95;
   SEQ ID NO: 96 and SEQ ID NO: 97;
   SEQ ID NO: 98 and SEQ ID NO: 99;
   SEQ ID NO: 100 and SEQ ID NO: 101;
   SEQ ID NO: 102 and SEQ ID NO: 103;
   SEQ ID NO: 104 and SEQ ID NO: 105;
   SEQ ID NO: 106 and SEQ ID NO: 107;
   SEQ ID NO: 108 and SEQ ID NO: 109;
   SEQ ID NO: 110 and SEQ ID NO: 111;
   SEQ ID NO: 112 and SEQ ID NO: 113;
   SEQ ID NO: 114 and SEQ ID NO: 115;
   SEQ ID NO: 116 and SEQ ID NO: 117;
   SEQ ID NO: 118 and SEQ ID NO: 119;
   SEQ ID NO: 120 and SEQ ID NO: 121;
   SEQ ID NO: 122 and SEQ ID NO: 123;
   SEQ ID NO: 124 and SEQ ID NO: 125;
   SEQ ID NO: 126 and SEQ ID NO: 127;
   SEQ ID NO: 128 and SEQ ID NO: 129;
   SEQ ID NO: 130 and SEQ ID NO: 131;
   SEQ ID NO: 132 and SEQ ID NO: 133;
   SEQ ID NO: 134 and SEQ ID NO: 135;
   SEQ ID NO: 136 and SEQ ID NO: 137;
   SEQ ID NO: 138 and SEQ ID NO: 139; and
   SEQ ID NO: 140 and SEQ ID NO: 141;
(b) the nucleic acid sequences in (a) wherein T can also be U;
(c) nucleic acid sequences complementary to (a) or (b); and
(d) nucleic acid sequences which are homologous to (a), (b) or (c).

In embodiments of the invention, the one or more nucleic acid molecules are arranged in distinct spots that are known or determinable locations within the array on the substrate. A spot refers to a region of target DNA attached to the substrate as a result of contacting a solution comprising target DNA with the substrate. Each spot can be sufficiently separated from each other spot on the substrate such that they are distinguishable from each other during the hybridization analysis. In an embodiment, there are at least 48 spots on the DNA microarray; one spot for each of the 48 PCR products generated by the 48 sets of primers disclosed herein which are used as target DNA. In another embodiment, the DNA microarray includes at least one spot for an expression level control as described herein above.

The substrate may be any solid support to which nucleic acids can be immobilized, such as a membrane, a glass support, a filter, a tissue culture dish, a polymeric material, a bead or a silica support. For example, the substrate can be a NoAb BioDiscoveries Inc. activated covalent-binding epoxy slide [UAS0005E].

When the nucleic acid molecule is immobilized on the substrate, a conventionally known technique can be used. For example, the surface of the substrate can be treated with polycations such as polylysines to electrostatically bind the target molecules through their charges on the surface of the substrate, and techniques to covalently bind the 5′-end of the target DNA to the substrate may be used. Also, a substrate that has linkers on its surface can be produced, and functional groups that can form covalent bonds with the linkers can be introduced at the end of the DNA to be immmobilized. Then, by forming a covalent bond between the linker and the functional group, the DNA and such can be immobilized.

Other methods of forming arrays of oligonucleotides, peptides and other polymer sequences with a minimal number of synthetic steps are known and may be used in the present invention. These methods include, but are not limited to, light-directed chemical coupling and mechanically directed coupling. See Pirrung et al., U.S. Pat. No. 5,143,854 and PCT Application No. WO 90/15070, Fodor et al., PCT Publication Nos. WO 92/10092 and WO 93/09668, which disclose methods of forming vast arrays of peptides, oligonucleotides and other molecules using, for example, light-directed synthesis techniques. See also, Fodor et al., Science, 251, 767-77 (1991). These procedures for synthesis of polymer arrays are now referred to as VLSIPS™ procedures. Using the VLSIPS™ approach, one heterogeneous array of polymers is converted, through simultaneous coupling at a number of reaction sites, into a different heterogeneous array.

Transcription indicators (targets) from a test sample that have been subjected to particular stringency conditions hybridize to the nucleic acid molecules (probes) on the array. One of skill in the art will appreciate that hybridization conditions may be selected to provide any degree of stringency. In an embodiment, hybridization is performed at low stringency [15-18 hrs at 37° C. in 500 mM sodium Phosphate pH 6.0, 1% SDS, 1% BSA, 1 mM EDTA] to ensure hybridization and then subsequent washes are performed at higher stringency [0.1×SSC ;0.1% SDS then 0.1×SSC then water] to eliminate mismatched hybrid duplexes. Successive washes may be performed at increasingly higher stringency until a desired level of hybridization specificity is obtained. Stringency can also be increased by addition of agents such as formamide. Hybridization specificity may be evaluated by comparison of hybridization to the test nucleic acid sequences with hybridization to the various controls that can be present (e.g., expression level controls (positive and negative), etc.).

The nucleic acids that do not form hybrid duplexes are washed away leaving the hybridized nucleic acids to be detected, typically through detection of an attached detectable label. After hybridization, the arrays are inserted into a scanner that can detect patterns of hybridization. These patterns are detected by detecting the labeled transcription indicator now attached to the array, for e.g., if the transcription indicator is fluorescently labeled, the hybridization data are collected as light emitted from the labeled groups. Comparison of the absolute intensities of an array hybridized to nucleic acids from a test sample with intensities produced from the various control samples provides a measure of the relative expression of the nucleic acids represented by each of the probes.

If the transcription indicator, for example cDNA, is fluorescently labeled, the fluorescence is detected and acquired using a fluorescence scanner, for example, a GSI Lumonics ScanArray Lite Microarray Analysis System, and the fluorescence intensity analyzed with specific quantitation and data processing software on a dedicated computer, for example, QuantArray and GeneLinker Gold. In an embodiment, the intensity of fluorescence increases with increased ABC transporter gene expression. If the transcription indicator, for example cDNA, is radiolabelled, then detection can be carried out using an RU image scanner and such, and the intensity of the radiation can be analyzed with a computer. In an embodiment, the intensity of the radiation increases with increased ABC transporter gene expression.

In further embodiments of the present invention, the methods of the invention further comprise (a) generating a set of expression data from the detection of the amount of hybridization; (b) storing the data in a database; and (c) performing comparative analysis on the set of expression data, thereby analyzing ABC transporter gene expression. The present invention also relates to a computer system comprising (a) a database containing information identifying the expression level of a set of genes comprising at least two ABC transporter genes; and b) a user interface to view the information.

(V) Drug Screening Assays

In one embodiment, the method of the invention has been used in a drug screening analysis. For example, a test sample was exposed to a chemical compound or a drug, and then ABC transporter gene expression was detected in the test sample using the methods of the invention. In an embodiment of the invention, ABC transporter expression was detected at various time intervals after the test sample was exposed to a compound or drug, for example every 2 hours after exposure for 24 hours. In a further embodiment, after the test sample was exposed to the chemical or drug, mRNA was extracted from the test sample and then cDNA was produced using the extracted mRNA. The cDNA was labeled and allowed to hybridize with the one or more nucleic acid molecules, wherein each one of the one or more nucleic acid molecules comprised a sequence that specifically hybridizes to one ABC transporter gene. The amount of hybridization was detected and compared with the amount of hybridization obtained with the test sample treated under the same conditions except that it had not been exposed to the compound or drug (i.e. a control sample). By performing this comparison, the effect of the drug or compound on the expression of each of the ABC transporter genes (whether it be increased, decreased or the same) was determined.

Therefore, the nucleic acid molecules and methods of the present invention can be used to perform drug-associated ABC transporter gene expression profiling. Such profiling will identify potential modulators of ABC transporter gene expression. Accordingly, in yet another embodiment of the invention, there is provided a method for screening compounds for their effect on the expression of one or more ABC transporter genes comprising:
(a) exposing a test sample to one or more compounds;
(b) providing a transcription indicator from the test sample;
(c) providing one or more nucleic acid sequences, each comprising a sequence that specifically hybridizes to one ABC transporter gene;
(d) allowing said transcription inhibitor to hybridize with said one or more nucleic acid sequences; and
(e) detecting an amount of hybridization of said transcription indicator with said one or more nucleic acid sequences,
   wherein the amount of hybridization is indicative of expression of the one or more ABC transporter genes.

In further embodiments of the invention the method for screening compounds for their effect on the expression of one or more ABC transporter genes further comprises the steps of
(f) quantitatively or qualitatively comparing the amount of hybridization detected in step (e) with the amount of hybridization of transcription indicators from a control sample, thereby determining the effect of the one or more compounds on the expression of the one or more ABC transporter genes.

The term “control sample” as used herein means a sample that has been treated under the same conditions as the test sample except that it has not been exposed to one or more compounds, drugs or other conditions that may have an effect on ABC transporter gene expression.

The term “compound” as used herein means any agent, including drugs, which may have an effect of ABC transporter gene expression and includes, but is not limited to, small inorganic or organic molecules: peptides and proteins and fragments thereof; carbohydrates, and nucleic acid molecules and fragments thereof. The compound may be isolated from a natural source or be synthetic. The term compound also includes mixtures of compounds or agents such as, but not limited to, combinatorial libraries and extracts from an organism.

The term “exposed” as used herein means that the sample has been brought into contact with the compound(s) using any method known in the art. For example, cells lines may be exposed to a compound by adding the compound(s) to the media used for cell storage, growth and/or washing. In a further example, the exposure may be effected by administering the compound(s) to a test subject using any known methods for administration, and the test sample is obtained from the subject, again using any known means.

In a further embodiment of the present invention there is provided a method for screening compounds for their effect on the expression of one or more ABC transporter genes comprising:
(a) preparing an ABC transporter gene expression profile, using a method of the invention, of a test sample that has been exposed to the compound;
(b) preparing an ABC transporter gene expression profile, using a method of the invention, of a control sample; and
(c) quantitatively or qualitatively comparing the gene expression profile in (a) and (b),
   wherein differential expression in (a) and (b) is indicative of a compound having an effect on the expression of one or more ABC transporter genes.

In yet another embodiment of the invention, the expression of one or more ABC transporter genes in the test and/or control samples is monitored over a set period of time and at specified time intervals to determine the effect of the compound on the expression of one or more ABC transporter genes over that period of time.

In embodiments of the invention, the methods may be used to identify compounds or agents that stimulate, induce and/or up-regulate the transcription or expression of one or more ABC transporter genes, or to down-regulate, suppress and/or counteract the transcription or expression of one or more ABC transporter genes, or that have no effect on transcription or expression of one or more ABC transporter genes, in a given system. According to the present invention, one can also compare the specificity of a compound's effect by looking at the number of ABC transporter genes, the expression of which has been effected. More specific compounds will have fewer transcriptional targets. Further, similar sets of results for two different compounds indicates a similarity of effects for the two compounds.

The ABC expression data can be used to design or choose an effective drug or chemical for the treatment of disease, such as cancer. By knowing which of the ABC transporter genes are modulated in the presence of the drug or compound, one can determine a cell's or patient's predisposition to drug toxicity and/or response to drug treatment. For example, if the chemical or drug up-regulates or increases the expression of certain ABC transporters in a test sample that are known to be involved in transporting compounds out of cells, for example ABC B1 (MDR1), ABC C1 (MRP1), ABC C2 (MRP2), or ABC G2 (BCRP), then the efficacy of that compound may be lowered. Further, if the compound down-regulates or decreases the expression of certain ABC transporters in a test sample that are known to be involved in transporting compounds out of cells, for example ABC B1 (MDR1), ABC C1 (MRP1), ABC C2 (MRP2), or ABC G2 (BCRP), then the efficacy and/or toxicity of that compound may be increased.

Accordingly the present invention further relates to a method of assessing the toxicity and/or efficacy of a compound comprising:
(a) preparing an ABC transporter gene expression profile, using a method of the invention, of a test sample that has been exposed to the compound;
(b) preparing an ABC transporter gene expression profile, using a method of the invention, of a control sample; and
(c) quantitatively or qualitatively comparing the ABC transporter gene expression profile from (a) and (b),
   wherein a difference in the ABC transporter gene expression profiles in (a) and (b) is indicative of the toxicity and/or efficacy of the compound.

In an embodiment of the invention, if the expression of one or more of the ABC transporter genes in the test sample is increased or induced by the compound(s), then the efficacy of the compound(s) may be decreased. For example, if the compound(s) increase or induce the expression of ABC B1 (MDR1), ABC C1 (MRP1), ABC C2 (MRP2), or ABC G2 (BCRP), then the efficacy of that compound may be lowered due to increased transport out of the cell. Conversely, if the expression of one or more of the ABC transporter genes in the test sample is decreased or suppressed by the compound(s), then the efficacy and/or the toxicity of the compound(s) may be increased. For example, if the compound(s) decrease or suppress the expression of ABC B1 (MDR1), ABC C1 (MRP1), ABC C2 (MRP2), or ABC G2 (BCRP), then the efficacy and/or toxicity of that compound may be increased due decreased transport out of the cell. This information is particularly important when designing drug treatments, including dosing amounts, for a particular disease.

In an embodiment of the invention, the compound is administered to a subject and ABC transporter gene expression in profiled in a test sample from the subject before and/or after administration of the compounds. Changes in ABC transporter gene expression are indicative of the toxicity and/or efficacy of the compound in the subject. In a further embodiment, the subject is human.

In a further embodiment, the nucleic acids and methods of the present invention are used to determine drug/drug interactions and their concomitant effect of ABC transporter gene expression. When two or more drugs are administered together, for example in combination therapy, ABC transporter gene expression may be altered. This is particularly relevant if two or more drugs are transported by the same transporter. What might be a non-toxic dose of a drug when administered on its own, may turn into a toxic dose when that drug is administered along with another drug, for example if both drugs are substrates for the same transporter. Therefore it is important to determine a drug's effect on ABC transporter gene expression alone, as well as in the presence of one or more other drugs with which it may be co-administered. Accordingly, in a further embodiment of the present invention there is provided a method for determining a change in ABC transporter gene expression profile for a compound in the presence of one or more different compounds comprising:
(a) preparing an ABC transporter gene expression profile, using a method of the invention, of a test sample that has been exposed to the compound;
(b) preparing an ABC transporter gene expression profile, using a method of the invention, of a test sample that has been exposed to the compound and the one or more different compounds; and
(c) quantitatively or qualitatively comparing the gene expression profile in (a) and (b),
   wherein differential expression in (a) and (b) indicates that ABC transporter gene expression profile of the compound changes in the presence of the one or more different compounds.

In an embodiment of the invention, differential expression indicates the presence of drug-drug interactions. If drug-drug interactions are found, then caution would need to be taken when determining effective drug therapies, including dosing, when the drugs are to be present in the body or cell at the same time.

The methods of the present invention may also be used to monitor the changes in ABC transporter gene expression profile as a function of disease state. For example, an ABC transporter gene expression profile of a test sample from the subject may be obtained at one point in time and again at a later date. Changes in ABC transporter gene expression profile are indicative of changes in disease state, treatment response or treatment toxicity.

Another embodiment of the invention is the use of the ABC transporter gene expression information for population profiling. For example, the ABC transporter gene expression information can be used to pre-selected individuals for clinical trials into non-responder and responder groups to a particular drug or chemical before initiation of the clinical trial.

(VI) Databases

The present invention also includes relational databases containing ABC transporter gene expression profiles in various tissue samples and/or cell lines. The database may also contain sequence information as well as descriptive information about the gene associated with the sequence information, the clinical status of the test sample and/or its source. Methods of configuring and constructing such databases are known to those skilled in the art (see for example, Akerblom et al. U.S. Pat. No. 5,953,727).

The databases of the invention may be used in methods to identify the expression level in a test sample of the ABC transporter genes by comparing the expression level at least one of the ABC transporter genes in the test sample with the level of expression of the gene in the database. Such methods may be used to assess the physiological state or a given test sample by comparing the level of expression of an ABC transporter gene or genes in the sample with that found in samples from normal, untreated samples or samples treated with other agents.

(VII) Kits

The present invention further includes kits combining, in different combinations, nucleic acid arrays or microarrays, reagents for use with the arrays, signal detection and array-processing instruments, gene expression databases and analysis and database management software described above. The kits may be used, for example, to predict or model the toxic or therapeutic response of a test compound, to monitor the progression of disease states, to identify genes that show promise as new drug targets and to screen known and newly designed drugs as discussed above.

The databases packaged with the kits are a compilation of expression patterns from human or laboratory animal ABC transporter genes. Data is collected from a repository of both normal and diseased animal tissues and provides reproducible, quantitative results, i.e., the degree to which a gene is up-regulated or down-regulated under a given condition.

The kits may used in the pharmaceutical industry, where the need for early drug testing is strong due to the high costs associated with drug development, but where bioinformatics, in particular gene expression informatics, is still lacking. These kits will reduce the costs, time and risks associated with traditional new drug screening using cell cultures and laboratory animals. The results of large-scale drug screening of pre-grouped patient populations, pharmacogenomics testing, can also be applied to select drugs with greater efficacy and fewer side-effects. The kits may also be used by smaller biotechnology companies and research institutes who do not have the facilities for performing such large-scale testing themselves.

Databases and software designed for use with use with microarrays is discussed in Balaban et al., U.S. Pat. No. Nos. 6,229,911, a computer-implemented method for managing information, stored as indexed tables, collected from small or large numbers of microarrays, and U.S. Pat. No. 6,185,561, a computer-based method with data mining capability for collecting gene expression level data, adding additional attributes and reformatting the data to produce answers to various queries. Chee et al., U.S. Pat. No. 5,974,164, disclose a software-based method for identifying mutations in a nucleic acid sequence based on differences in probe fluorescence intensities between wild type and mutant sequences that hybridize to reference sequences.

(VIII) Methods of Conducting Drug Discovery Businesses

Yet another aspect of the present invention provides a method of conducting a target discovery business comprising:
(a) providing one or more assay systems for identifying agents by their ability to modulate ABC transporter gene expression, said assay systems using a method of the invention;
(b) (optionally) conducting therapeutic profiling of agents identified in step (a) for efficacy and toxicity in animals; and
(c) licensing, to a third party, the rights for further drug development and/or sales or agents identified in step (a), or analogs thereof.

By assay systems, it is meant, the equipment, reagents and methods involved in conducting a screen of compounds for the ability to modulate ABC transporter gene expression using the method of the invention.

The following non-limiting examples are illustrative of the present invention:

### EXAMPLES

### Example 1

### Sets of Primers and Resulting PCR Products for Each ABC Transporter Gene

The sets of primers were designed such that the amplification product is a PCR amplicon that is a unique portion of an ABC transporter gene (See table 1). FIGS. 1 to 47 show nucleic acid sequences for each PCR amplicon. The primers are shown in bold.

The NCBI (www.ncbi.nim.nig.gov) and BCM search launcher (www.searchlauncher.bcm.tme.edu) websites were used to verify PCR primer identity with the ABC transporter gene region of interest. BLAST sequence searches and alignment analyses were completed for each PCR primer pair and PCR amplicon to ensure minimum cross-hybridization with other known genes and other known ABC transporter genes.

Total RNA Preparation

Cell lines were grown as adherent monolayers following the ATCC guidelines in Falcon T175 flasks until semi-confluent. Culture medium was removed. The adherent cells were washed twice with PBS (phosphate buffered saline) pH7.4. 1.6 ml TriZol reagent (Cat. No. 15596-018, Invitrogen Life Technologies) was added to each flask to lyse the cells and liberate the nucleic acids. The total RNA component of the nucleic acid lysate was isolated according to the manufacturer's instructions. Total RNA was quantitated by spectrophotometric analysis and OD_{260 nm}:OD_{280 nm} ratios.

cDNA Synthesis

cDNA was prepared from 20 μg of total RNA in a total volume of 40 μl. 20 μg of total RNA was added to a 200 μl RNase-free microtube and placed on ice. 4 μl of a 300 ng/μl solution of random d(N)g primers (Cat. No. S1254S, New England BioLabs) was added to the tube containing the total RNA and the final volume made up to 22 μl with RNase-free dH₂O. The microtube was capped and then heated at 65° C. for 10 min in a thermal cycler (PTC200 DNA Engine, MJ Research). The microtube was then removed from the thermal cycler and placed on ice for 3min. The microtube was spun in a microfuge (C-1200, VWR Scientific Products) to collect the solution in the bottom of the microtube and placed on ice.

First-strand cDNA synthesis was accomplished with the SuperScript II RNase H-Reverse Transcriptase reagent set (Cat. No. 18064-014, Invitrogen Life Technologies). 8 ul 5× First-Strand Buffer [250 mM Tris-HCl pH 8.3, 375 mM KCl, 15 mM MgCl₂], 4 μl 100 mM DTT, 2 μl 10 mM dNTP Mix [10 mM each dATP, dCTP, dGTP, dTTP] were added to the microtube on ice. The microtube was capped and then heated at 25° C. for 10 min in a thermal cycler. The microtube was then heated at 42° C. for 2 min in a thermal cycler. The microtube was uncapped and left in the thermal cycler. 2 μl SuperScript II (200 U/μl) was added to the solution in the microtube and mixed with the micropipette tip. The microtube was recapped and incubated at 42° C. for 60 min in a thermal cycler. Subsequent to this incubation the microtube was heated at 70° C. for 15 min in a thermal cycler. The microtube was then removed from the thermal cycler and spun in a microfuge to collect the solution in the bottom of the microtube and then returned to the thermal cycler. 1 μl of RNase H (2 U/μl) was added to the cDNA synthesis reaction and incubated at 37° C. for 20 min in a thermal cycler. The first-strand cDNA synthesis reaction was then stored at −20° C. until required for RT-PCR.

RT-PCR

RT-PCR was performed in a final volume of 25 μl. 2 μl of the first-strand cDNA synthesis reaction was added to a 200 μl microtube and placed on ice. 2 μl of a specific ABC Drug Transporter (ABC-DT) primer pair mix [10 μM each forward PCR primer and reverse PCR primer], 2.5 μl 10×PCR Buffer [200 mM Tris-HCl pH 8.4, 500 mM KCl], 0.75 μl 50 mM MgCl₂, 0.5 μl 10 mM dNTP Mix [10 mM each dATP, dCTP, dGTP, dTTP], 16.25 μl dH₂O and 1 μl Taq polymerase (5U/ul) were added to the side of the microtube. The reagents were mixed and collected in the bottom of the microtube by spinning the capped microtube in a microfuge. The capped microtube was then placed in a thermal cycler block with a heated lid (PTC200 DNA Engine, MJ Research), both pre-heated to 95° C., and incubated at this temperature for 5 min. After this initial denaturation step 40 cycles of PCR amplification were performed as follows: Denature 95° C. for 30 s, Anneal 60° C. for 30 s, Extend 72° C. for 60 s. Following the final 72° C. Extend step the PCR was incubated for an additional 10 min at 72° C. The PCR was then maintained at a temperature of 15° C. PCR products were stored at −20° C. until needed.

PCR Amplicon Purification

ABC-DT RT-PCR amplification products (PCR amplicons) were analysed by electrophoresis at 150V for 20 min in 1×TAE running buffer in an agarose gel [0.8% agarose, 1×TAE, 0.5 μg/ml ethidium bromide] with 4 μl of a 250 bp DNA Ladder (Cat. No. 10596-013, Invitrogen Life Technologies) to permit size estimates of the PCR amplicons.

The ABC-DT RT-PCR amplification products (PCR amplicons) were visualised “in gel” with a UV transilluminator (UVP M-15, DiaMed Lab Supplies) and photographed with a photo-documentation camera and hood (FB-PDC-34, FB-PDH-1216, Fisher Biotech), a #15 Deep Yellow 40.5 mm screw-in optical glass filter (FB-PDF-15, Fisher Biotech) and Polaroid Polapan 667 film.

The ABC-DT RT-PCR amplification products (PCR amplicons) were isolated and purified from the ABC-DT RT-PCR using the QIAquick PCR purification kit (Cat. No. 28104, QIAGEN Inc.) according to the manufacturer's instructions. After purification, ABC-DT RT-PCR amplification products (PCR amplicons) were analysed by electrophoresis at 150V for 20 min in 1×TAE running buffer in an agarose gel [0.8% agarose, 1×TAE, 0.5 ug/ml ethidium bromide] with 4 μl of a Low DNA Mass Ladder (Cat. No. 10068-013, Invitrogen Life Technologies) to permit PCR amplicon sizing and quantitation.

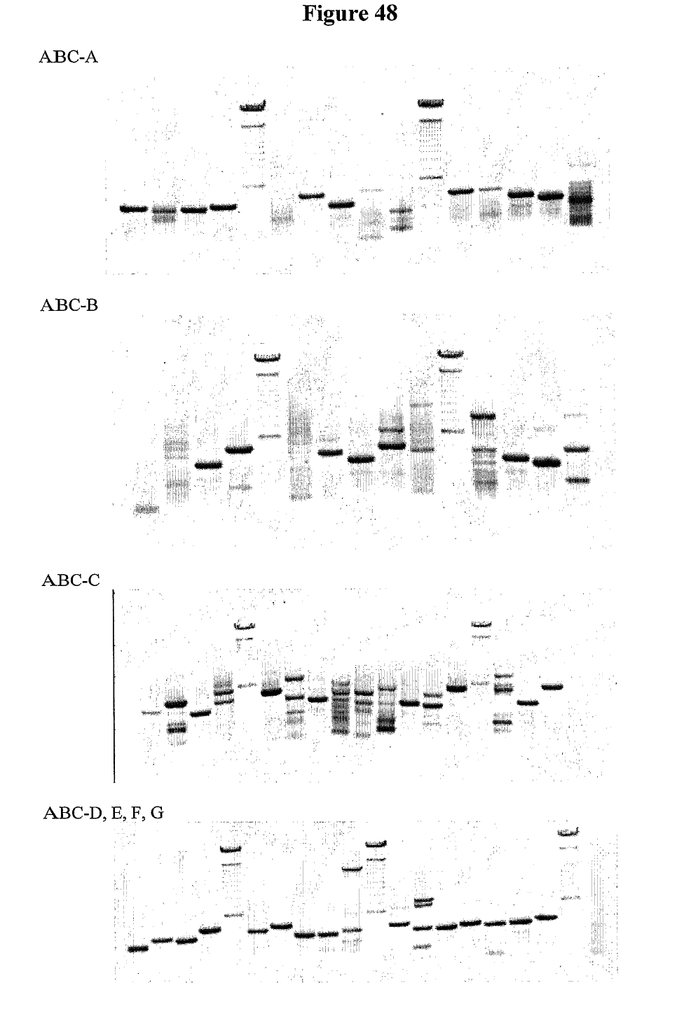

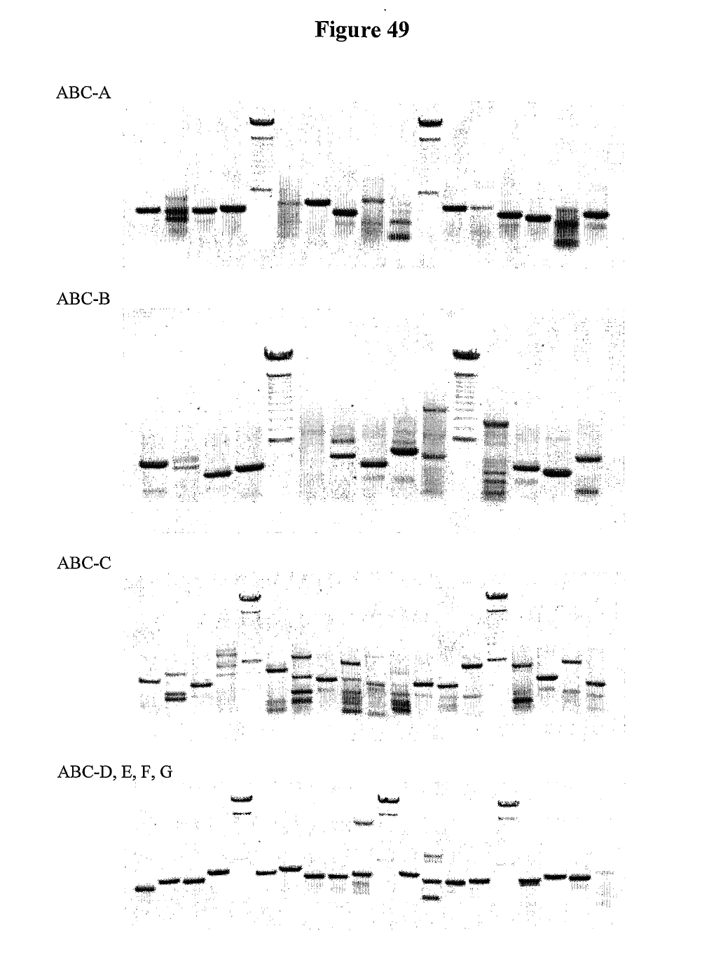

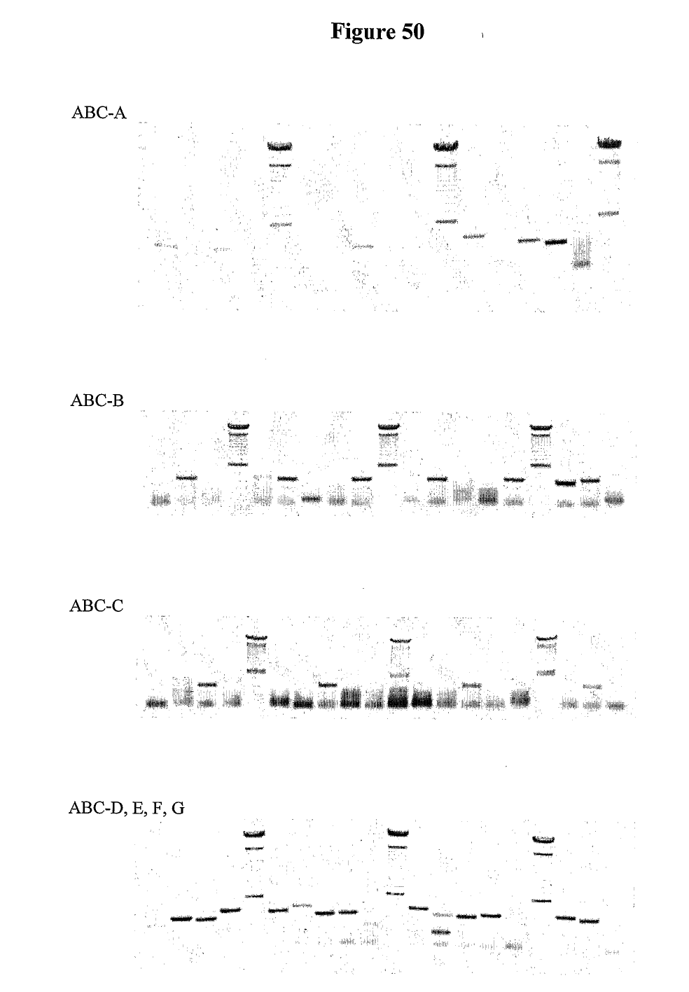

FIG. 48 shows the ABC transporter gene RT-PCR amplification products from the CaCo2 cell line. FIG. 49 shows the ABC transporter gene RT-PCR amplification products from the HEK293 cell line. FIG. 50 shows the ABC transporter gene RT-PCR amplification products from the HepG2 cell line.

### Example 2

### Sequencing

The sequences of the PCR amplicons, which are each unique portions of each of the known human ABC transporter genes, can be verified.

ABC-DT PCR Amplicon Cloning and Sequencing

A number of the purified ABC-DT RT-PCR amplification products (PCR amplicons) were cloned into pCR4-TOPO vectors using the TOPO TA Cloning Kit for Sequencing (Cat. No. K4575-40, Invitrogen Life Technologies) according to the manufacturer's instructions to verify the sequence of the purified ABC-DT PCR amplicon.

DNA sequence analysis was performed with Cy5.5-labelled M13 (−20) universal and M13 reverse primers, the Cy5/Cy5.5 Dye Primer Cycle Sequencing Kit (Cat. No. VG 30001, Visible Genetics Inc./Bayer Inc.) and the OpenGene automated DNA sequencing system (MGB-16, Visible Genetics Inc./Bayer Inc.) according to the manufacturer's instructions.

### Example 3

### DNA Microarray

ABC-DT Microarray (DT1 Microarray)

1-2 μg of each of the purified ABC-DT RT-PCR amplification products (PCR amplicons) and 5 purified positive control RT-PCR amplification products (PCR amplicons) were aliquoted into individual wells of a CoStar SeroCluster 96 well U-bottom polypropylene microwell plate (source plate). The source plate was placed in a Speed-Vac concentrator (SPD101B, Savant Instruments Inc.) and dried under vacuum for 1 hour at 45° C. The dry RT-PCR amplification products (PCR amplicons) in the source plate were resuspended in 20 μl 1×NoAb Print Buffer (150 mM sodium phosphate pH 8.5, Cat. No. UAS0001PB, NoAb BioDiscoveries Inc.), sealed with mylar sealing tape (Cat. No. T-2162, Sigma Chemical Company) and dissolved by shaking at 300 rpm for 1 hour at room temperature on a microplate shaker (EAS2/4, SLT Lab Instruments).

The source plate was then placed in a humidified (21-25° C., 45-60% RH) microarrayer cabinet (SDDC-2, ESI/Virtek Vision Corp./BioRad Laboratories Inc.). Each purified RT-PCR amplification product (PCR amplicon) was printed in quadruplicate on activated covalent-binding epoxy slides (Cat. No. UAS0005E, NoAb BioDiscoveries Inc.) using Stealth micro-spotting pins (Cat. No. SMP5, TeleChem International Inc.). The 384 element microarrays were air-dried in the microarrayer cabinet for at least 4 hours. Printed microarrays were stored in 20 slide racks under vacuum until needed.

### Example 4

### Method for Detecting ABC Transporter Gene Expression Using a DNA Microarray

The ABC transporter gene expression profile for 22 different cell lines was prepared using the DNA microarray.

Total RNA Preparation

All 22 cell lines (BT20, CaCo2, CaOv, Colo320, HBT161, HEK293, HepG2, HT75, HT177, LnCaP, MCF7, MDA453, MDA468, MFE29C, SKMES1, SKNAS, SKNBE, SKND2, SKNMC, T47D, ZR75, MDCK) were grown as adherent monolayers following the ATCC guidelines in tissue culture flasks until semi-confluent. Culture medium was removed. The adherent cells were washed twice with PBS (phosphate buffered saline) pH7.4. 1.6 ml TriZol reagent (Cat. No. 15596-018, Invitrogen Life Technologies) was added to each flask to lyse the cells and liberate the nucleic acids. The total RNA component of the nucleic acid lysate was isolated according to the manufacturer's instructions. Total RNA was quantitated by spectrophotometric analysis and OD_{260 nm}:OD₂₈₀ nm ratios.

Fluorescent cDNA Target Preparation

Fluorescently labelled cDNA targets were prepared from each of the 22 cell lines using 20 μg of total RNA in a total volume of 40 μl.

20 μg of total RNA was added to a 200 μl RNase-free microtube and placed on ice. 4 μl of a 300 ng/μl solution of random d(N)₉ primers (Cat. No. S1254S, New England BioLabs) was added to the tube containing the total RNA and the final volume made up to 22 μl with RNase-free dH₂O. The microtube was capped and then heated at 65° C. for 10 min in a thermal cycler (PTC200 DNA Engine, MJ Research). The microtube was then removed from the thermal cycler and placed on ice for 3 min. The microtube was spun in a microfuge (C-1200, VWR Scientific Products) to collect the solution in the bottom of the microtube and placed on ice.

First-strand cDNA synthesis was accomplished with the SuperScript II RNase H-Reverse Transcriptase reagent set (Cat. No. 18064-014, Invitrogen Life Technologies). 8 μl 5× First-Strand Buffer [250 mM Tris-HCl pH 8.3, 375 mM KCl, 15 mM MgCl₂], 4 μl 100 mM DTT, 2 μl T-dNTP Mix [2.3 mM dTTP, 5 mM each dATP, dCTP, dGTP], 2 μl ChromaTide Alexa 546-14-dUTP (1 mM in TE buffer, Cat. No. C-11401, Molecular Probes Inc.) were added to the microtube on ice. The microtube was capped and then heated at 25° C. for 10 min in a thermal cycler. The microtube was then heated at 42° C. for 2 min in a thermal cycler. The microtube was uncapped and left in the thermal cycler. 2 ul SuperScript II (200 U/μl) was added to the solution in the microtube and mixed with the micropipette tip. The microtube was recapped and incubated at 42° C. for 60 min in a thermal cycler. Subsequent to this incubation the microtube was heated at 70° C. for 15 min in a thermal cycler. The microtube was then removed from the thermal cycler and spun in a microfuge to collect the solution in the bottom of the microtube and then returned to the thermal cycler. 1 μl of RNase H (2 U/μl) was added to the cDNA synthesis reaction and incubated at 37° C. for 20 min in a thermal cycler. The fluorescently labelled cDNA targets were stored at −20° C. overnight before QIAquick column purification.

The fluorescently labelled cDNA targets were thawed and the total volume adjusted to 100 μl with dH₂O. Labelled cDNA targets were isolated and purified using the QIAquick PCR purification kit (Cat. No. 28104, QIAGEN Inc.) according to the manufacturer's instructions except that the final elution volume was adjusted to 150 μl. The purified cDNA target preparation was stored at −20° C. until required for microarray hybridisation.

DT1 Microarray Hybridisation

The printed DT1 microarray(s) was removed from storage under vacuum and placed in a 20 slide rack. The DT1 microarray was then denatured by dipping the microarray slide into “boiled” dH₂O for 30 s. The denatured DT1 microarray was then placed in a polypropylene 5 slide mailer (Cat. No. 240-3074-030, Evergreen Scientific) and blocked in 1×NoAb Pre-Hybridisation Blocking Buffer (Cat. No. UAS0001BB, NoAb BioDiscoveries Inc.) for 2 hours at room temperature. Pre-hybridised, blocked DT1 microarrays were removed from this solution and placed in a new polypropylene 5 slide mailer (Cat. No. 240-3074-030, Evergreen Scientific) containing a solution of denatured, labelled cDNA targets from a specific cell line.

The labelled cDNA target preparation was thawed and the 150 μl added to 850 μl hybridisation buffer (500 mM sodium Phosphate pH 6.0, 1% SDS, 1% BSA, 1 mM EDTA) in a 1.5 ml microtube and heated at 95° C. for 10 min. Following denaturation the microtube was spun briefly in a microcentrifuge to collect all the liquid. The denatured, labelled cDNA targets were then added to a polypropylene 5 slide mailer (Cat. No. 240-3074-030, Evergreen Scientific) that contained a pre-hybridised, blocked DT1 microarray placed “array-side” down in the bottom-most slot of the 5 slide mailer. In this orientation the entire surface of the microarray slide is bathed in the hybridisation buffer. 5 slide mailers containing the DT1 microarrays were incubated on their sides, “array-side” down, in a 37° C. incubator for 15-18 h.

Hybridised DT1 microarrays were removed from the 5 slide mailers with forceps and placed directly into a 20 slide rack in a slide wash box containing a 0.1×SSC, 0.1% SDS solution. DT1 microarrays were incubated in this solution at 37° C. for 15 min. The slide rack containing the DT1 microarrays was then transferred to a slide wash box containing 0.1×SSC and incubated in this solution at 37° C. for 15 min. Following this step the DT1 microarrays were rinsed in dH₂O and air-dried by centrifugation at 1200 rpm.

DT1 Microarray Image Acquisition and Data Analysis

Processed DT1 microarrays were scanned using ScanArray software in a ScanArray Lite MicroArray Analysis System (GSI Lumonics Inc.) at a scan resolution of 10 μm, a laser setting of 90 and a PMT gain of 80. Images were analysed using QuantArray software (GSI Lumonics Inc.). The data generated from QuantArray was exported to GeneLinker Gold (Molecular Mining Inc./Predictive Patterns Software) for bioinformatic analysis and data mining. Gene expression profiles and hierarchical clustering maps (“heat maps”) were also generated using GeneLinker Gold.

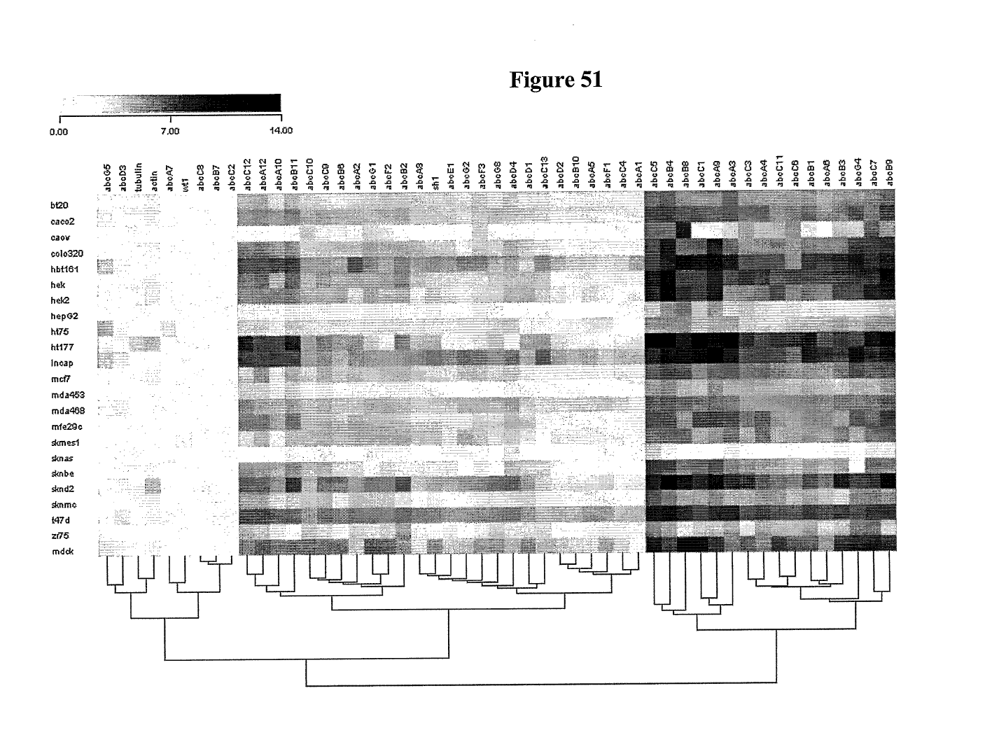

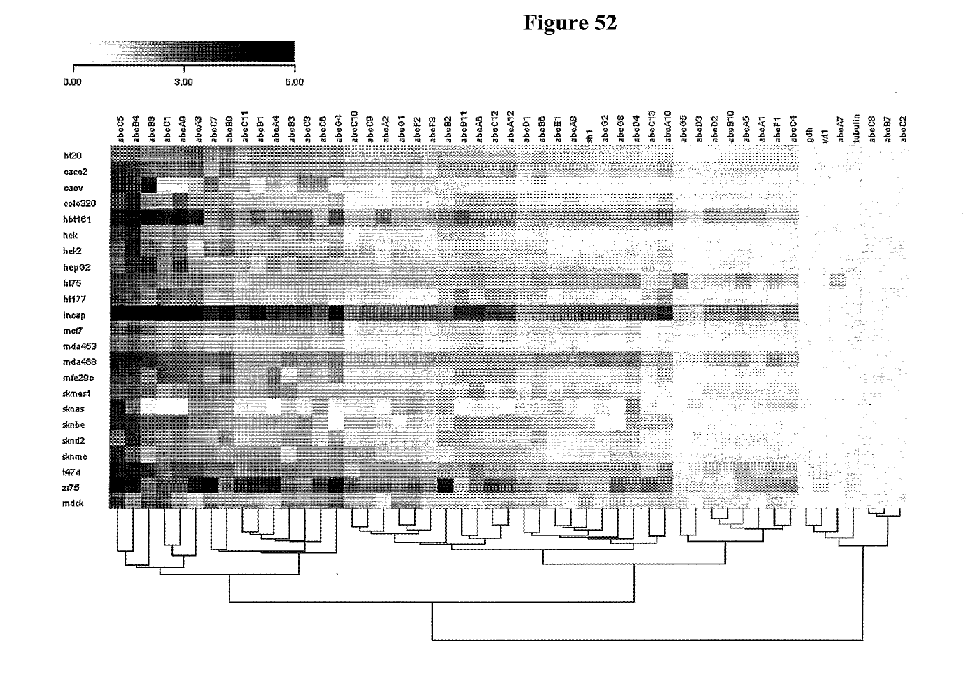

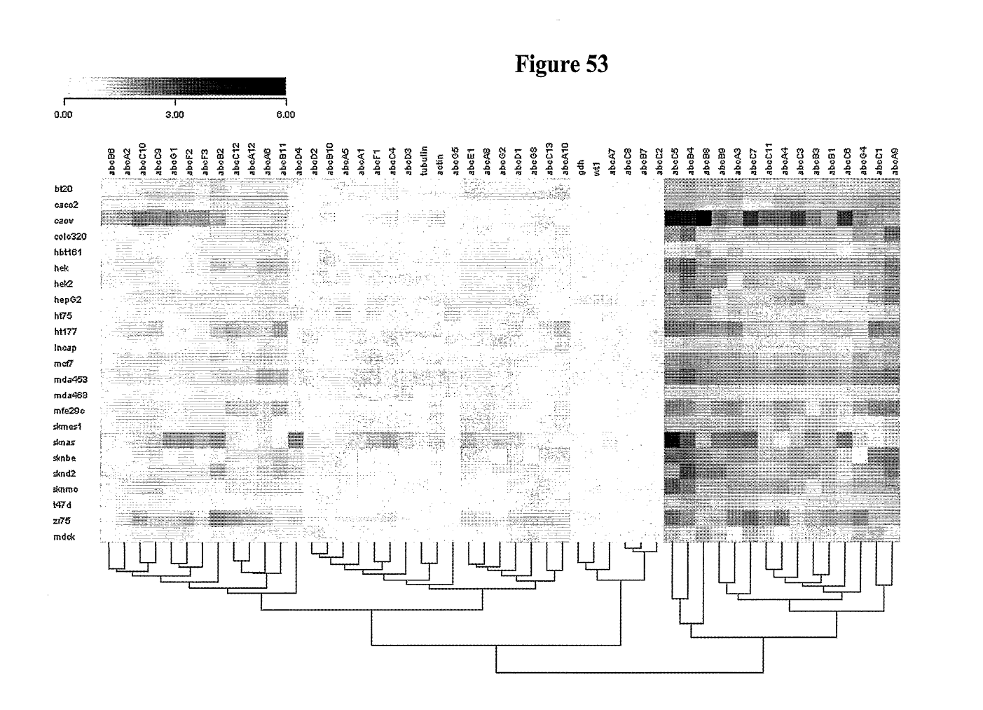

FIG. 51 shows the fluorescence intensity cluster plot for and Table 2 sets out the relative levels of ABC transporter gene expression in various cell lines normalized to GAPDH. FIG. 52 shows the fluorescence intensity cluster plot for and Table 3 sets out the relative levels of ABC transporter gene expression in various cell lines normalized to actin. FIG. 53 shows the fluorescence intensity cluster plot for and Table 4 sets out the relative levels of ABC transporter gene expression in various cell lines normalized to SH1.

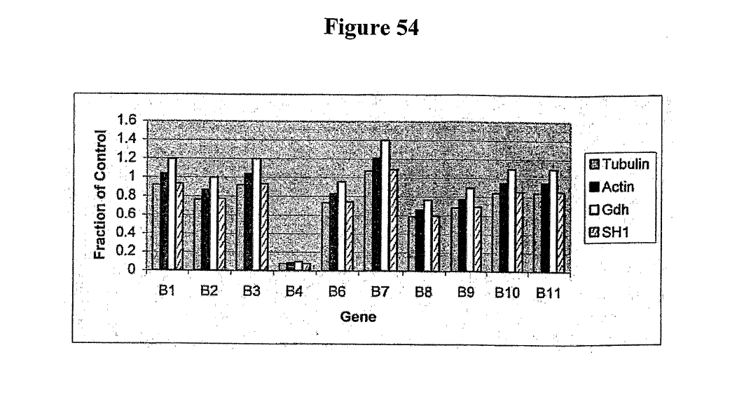

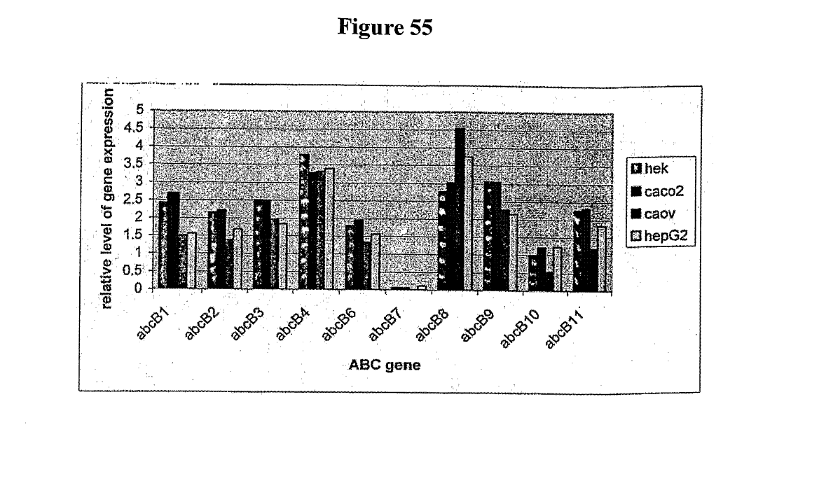

FIG. 54 shows the relative levels of gene expression for ABC B1 to B11 in HEK cells normalized to constitutively expressed control genes (tubulin, actin, GAPDH, and SH1). FIG. 55 shows the relative levels of gene expression for ABC B1 to B11 in various cell lines (HEK, CaCo2, CaOv and HepG2) normalized to the constitutively expressed actin control gene.

As shown in FIG. 55, the ABC transporter gene expression profile is different for different cell lines. Certain ABC transporter genes are over-expressed in some cell lines, while some are suppressed in other cell lines.

### Example 5

### Drug Screening Assay

Cell lines were treated with two chemotherapeutic agents, doxorubicin and vinblastine, at 2 hour intervals.

Total RNA Preparation From Drug-Treated HeDG2 Cell Line

The HepG2 cell line was grown as an adherent monolayer in 24 Falcon T175 flasks following the ATCC guidelines until semi-confluent. Tissue culture flasks were then divided into pairs for each of six timepoints (0 h, 2 h, 4 h, 8 h, 18 h, 24 h).

For vinblastine sulfate treatment, 5 μl of a 1000× (5 mM in DMSO) stock solution of vinblastine sulfate was added to 10 Falcon T175 flasks containing the HepG2 monolayer in 10 mls of culture medium (25 nM final concentration), mixed gently by rocking, returned to the CO₂ incubator and harvested for total RNA at the indicated times. The 0 h timepoint flasks were processed immediately after the addition of 5 μl DMSO.

For doxorubicin HCl treatment, 5 μl of a 1000× (5 mM in DMSO) stock solution of doxorubicin HCl was added to 10 Falcon T175 flasks containing the HepG2 monolayer in 10 mls of culture medium (25 nM final concentration), mixed gently by rocking, returned to the CO₂ incubator and harvested for total RNA at the indicated times. The 0 h timepoint flasks were processed immediately after the addition of 5 μl DMSO.

Prior to cell lysis the tissue culture medium was removed. The adherent cells were washed twice with PBS (phosphate buffered saline) pH7.4. 1.6 ml TriZol reagent (Cat. No. 15596-018, Invitrogen Life Technologies) was added to each flask to lyse the cells and liberate the nucleic acids. The total RNA component of the nucleic acid lysate was isolated according to the manufacturer's instructions. Total RNA was quantitated by spectrophotometric analysis and OD_{260 nm}:OD_{280 nm} ratios.

Fluorescent cDNA Target Preparation

Fluorescently labelled cDNA targets were prepared from each of the 12 timepoint samples for the drug-treated HepG2 cell line (6× vinblastine sulfate, 6× doxorubicin HCl) using 20 μg of total RNA in a total volume of 40 μl.

20 μg of total RNA was added to a 200 ul RNase-free microtube and placed on ice. 4 μl of a 300 ng/ul solution of random d(N)₉ primers (Cat. No. S1254S, New England BioLabs) was added to the tube containing the total RNA and the final volume made up to 22 μl with RNase-free dH₂O. The microtube was capped and then heated at 65° C. for 10 min in a thermal cycler (PTC200 DNA Engine, MJ Research). The microtube was then removed from the thermal cycler and placed on ice for 3 min. The microtube was spun in a microfuge (C-1200, VWR Scientific Products) to collect the solution in the bottom of the microtube and placed on ice.

First-strand cDNA synthesis was accomplished with the SuperScript II RNase H-Reverse Transcriptase reagent set (Cat. No. 18064-014, Invitrogen Life Technologies). 8 μl 5× First-Strand Buffer [250 mM Tris-HCl pH 8.3, 375 mM KCl, 15 mM MgCl₂], 4 μl 100 mM DTT, 2 ul T-dNTP Mix [2.3 mM dTTP, 5 mM each dATP, dCTP, dGTP], 2 μl ChromaTide Alexa 546-14-dUTP (1 mM in TE buffer, Cat. No. C-11401, Molecular Probes Inc.) were added to the microtube on ice. The microtube was capped and then heated at 25° C. for 10 min in a thermal cycler. The microtube was then heated at 42° C. for 2 min in a thermal cycler. The microtube was uncapped and left in the thermal cycler. 2 μl SuperScript II (200 U/μl) was added to the solution in the microtube and mixed with the micropipette tip. The microtube was recapped and incubated at 42° C. for 60 min in a thermal cycler. Subsequent to this incubation the microtube was heated at 70° C. for 15 min in a thermal cycler. The microtube was then removed from the thermal cycler and spun in a microfuge to collect the solution in the bottom of the microtube and then returned to the thermal cycler. 1 μl of RNase H (2 U/μl) was added to the cDNA synthesis reaction and incubated at 37° C. for 20 min in a thermal cycler. The fluorescently labelled cDNA targets were stored at −20° C. overnight before QIAquick column purification.

The fluorescently labelled cDNA targets were thawed and the total volume adjusted to 100 μl with dH₂O. Labelled cDNA targets were isolated and purified using the QIAquick PCR purification kit (Cat. No. 28104, QIAGEN Inc.) according to the manufacturer's instructions except that the final elution volume was adjusted to 150 μl. The purified cDNA target preparation was stored at −20° C. until required for microarray hybridisation.

DT1 Microarray Hybridisation

The printed DT1 microarray(s) was removed from storage under vacuum and placed in a 20 slide rack. The DT1 microarray was then denatured by dipping the microarray slide into “boiled” dH₂O for 30 s. The denatured DT1 microarray was then placed in a polypropylene 5 slide mailer (Cat. No. 240-3074-030, Evergreen Scientific) and blocked in 1×NoAb Pre-Hybridisation Blocking Buffer (Cat. No. UAS0001BB, NoAb BioDiscoveries Inc.) for 2 hours at room temperature. Pre-hybridised, blocked DT1 microarrays were removed from this solution and placed in a new polypropylene 5 slide mailer (Cat. No. 240-3074-030, Evergreen Scientific) containing a solution of denatured, labelled cDNA targets from a specific cell line.

The labelled cDNA target preparation was thawed and the 150 μl added to 850 ul hybridisation buffer (500 mM sodium Phosphate pH 6.0, 1% SDS, 1% BSA, 1 mM EDTA) in a 1.5 ml microtube and heated at 95° C. for 10 min. Following denaturation the microtube was spun briefly in a microcentrifuge to collect all the liquid. The denatured, labelled cDNA targets were then added to a polypropylene 5 slide mailer (Cat. No. 240-3074-030, Evergreen Scientific) that contained a pre-hybridised, blocked DT1 microarray placed “array-side” down in the bottom-most slot of the 5 slide mailer. In this orientation the entire surface of the microarray slide is bathed in the hybridisation buffer. 5 slide mailers containing the DT1 microarrays were incubated on their sides, “array-side” down, in a 37° C. incubator for 15-18 h.

Hybridised DT1 microarrays were removed from the 5 slide mailers with forceps and placed directly into a 20 slide rack in a slide wash box containing a 0.1×SSC, 0.1% SDS solution. DT1 microarrays were incubated in this solution at 37° C. for 15 min. The slide rack containing the DT1 microarrays was then transferred to a slide wash box containing 0.1×SSC and incubated in this solution at 37° C. for 15 min. Following this step the DT1 microarrays were rinsed in dH₂O and air-dried by centrifugation at 1200 rpm.

DT1 Microarray Image Acquisition and Data Analysis

Processed DT1 microarrays were scanned using ScanArray software in a ScanArray Lite MicroArray Analysis System (GSI Lumonics Inc.) at a scan resolution of 10 μm, a laser setting of 90 and a PMT gain of 80. Images were analyzed using QuantArray software (GSI Lumonics Inc.). The data generated from QuantArray was exported to GeneLinker Gold (Molecular Mining Inc./Predictive Patterns Software) for bioinformatic analysis and data mining. Gene expression profiles and hierarchical clustering maps for drug treatment-related changes in ABC-DT gene expression were also generated using GeneLinker Gold.

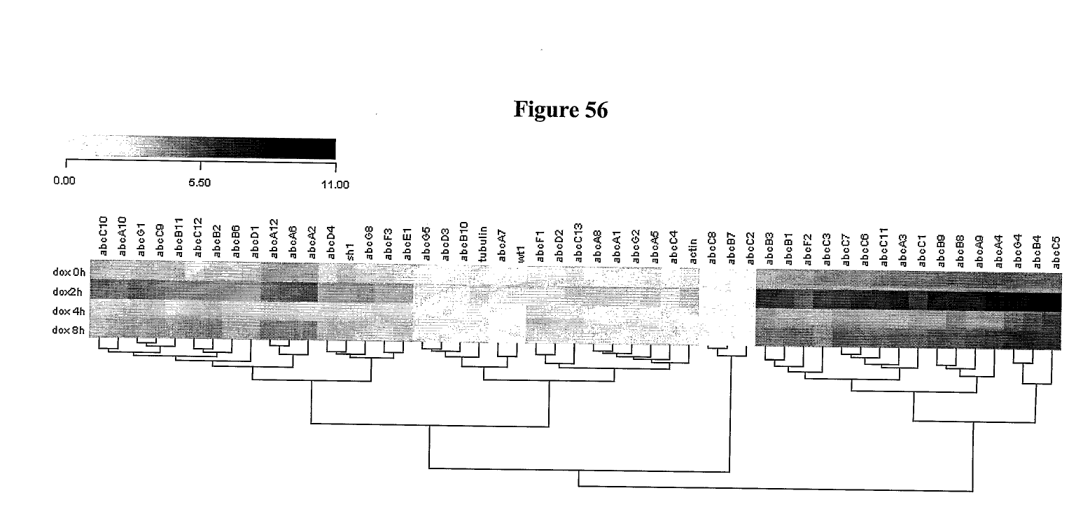

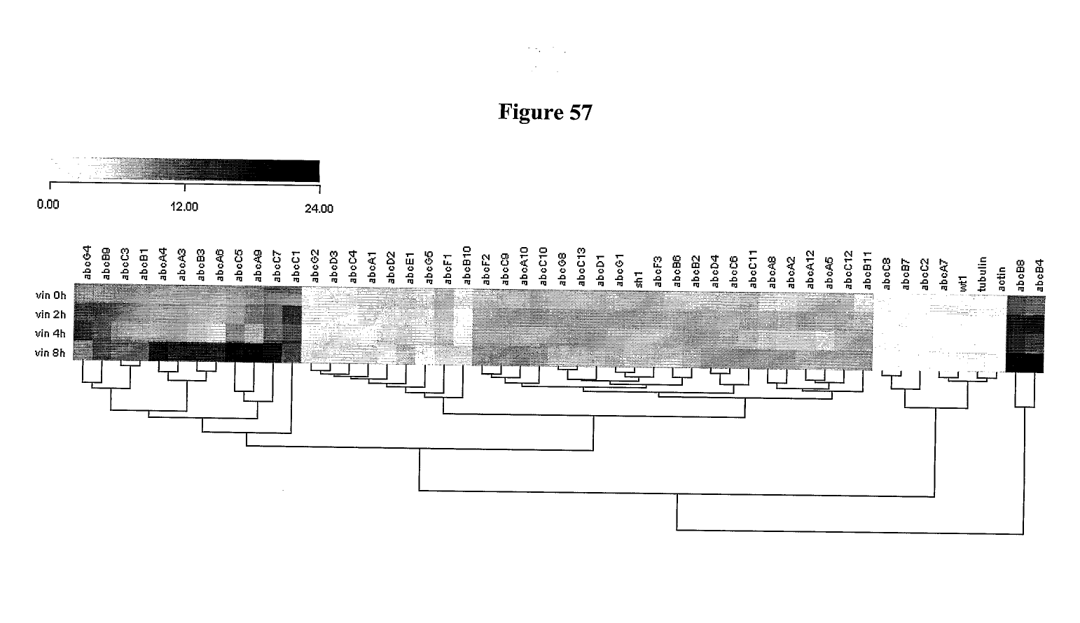

FIG. 56 shows the fluorescence intensity cluster plot for and Table 5 shows the relative levels of ABC transporter gene expression in cell lines treated with doxorubicin at various time intervals. FIG. 57 shows the fluorescence intensity cluster plot for and Table 6 shows the relative levels of ABC transporter gene expression in cell lines treated with vinblastine at various time intervals.

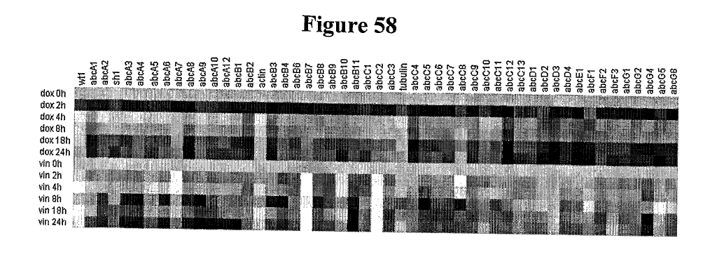

FIG. 58 shows a matrix plot of the relative levels of ABC transporter gene expression in a cell line [HepG2] treated with either doxorubicin [dox] or vinblastine [vin] at various time intervals.

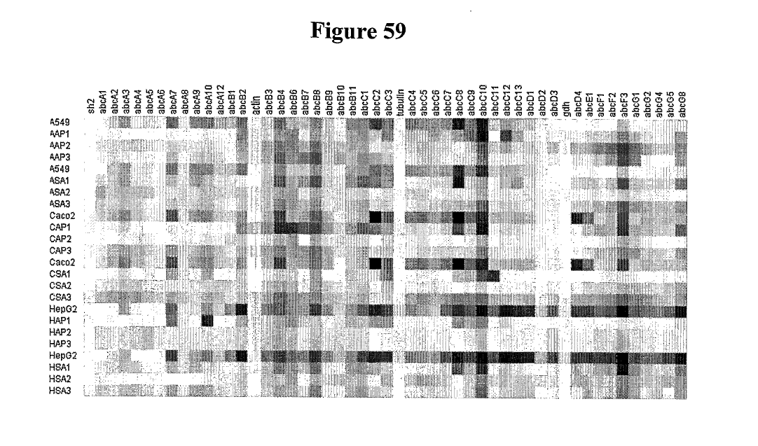

FIG. 59 shows a matrix plot of the relative levels of ABC transporter gene expression in several cell lines [A549, CaCo2, HepG2] treated with either acetaminophen [AP] or acetylsalicylic acid [SA].

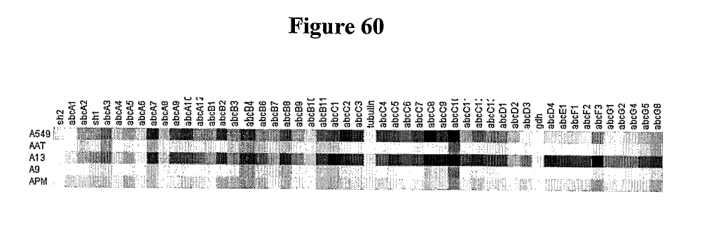

FIG. 60 shows a matrix plot of the relative levels of ABC transporter gene expression in a cell line [A549] treated with either all-trans retinoic acid [AAT], cis-13 retinoic acid [A13], cis-9 retinoic acid [A9] or phorbol-12-myristate-13-acetate [APM].

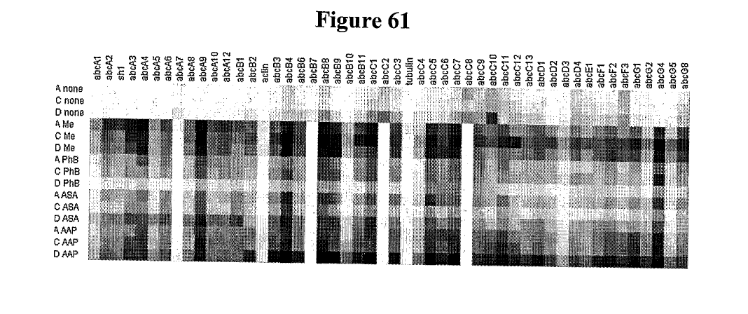

FIG. 61 shows a matrix plot of the relative levels of ABC transporter gene expression in cell lines HTB81 [A], CRL1740 [C] and CRL2505 [D] treated with either no drug [none], methanol [Me], phenobarbitol [PhB], acetylsalicylic acid [ASA] or acetaminophen [AAP].

While the present invention has been described with reference to what are presently considered to be examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1

Unique Portion
of ABC
Transporter

| | | |
|---|---|---|
| Gene | Upper Primer | Lower Primer |

| | | | | |
|---|---|---|---|---|
| ABCA1 | SEQ ID NO: 48 | 5′ CCC TGT GGA | SEQ ID NO: 49 | 5′ GCG TAA AGT |
| | | ATG TAC CTA | | GCT TGG AAT |
| | | TGT GAG 3′ | | GAG GGC 3′ |
| ABCA2 | SEQ ID NO: 50 | 5′ CCT TCA ACA | SEQ ID NO: 51 | 5′ AGC TTC TCC |
| | | CGG ACA CGC | | ATT CCT GCC |
| | | TCT GCT 3′ | | ACC TGC 3′ |
| ABCA3 | SEQ ID NO: 52 | 5′ AAG GAA AAG | SEQ ID NO: 53 | 5′ CTA AGA CCC |
| | | TAC GGC GTG | | CAG CAC CTA |
| | | GAC GAC 3′ | | ATC ACA 3′ |
| ABCA4 | SEQ ID NO: 54 | 5′ GAG CAT CAT | SEQ ID NO: 55 | 5′ GGG TTT CTA |
| | | CAG AAA AGG | | GTT CTG GGG |
| | | GAG GGC 3′ | | TCT GGA 3′ |
| ABCA5 | SEQ ID NO: 56 | 5′ AAT GCA AGC | SEQ ID NO: 57 | 5′ CTT ACA CTT |
| | | CGT CAG GAA | | CAG CTT TTA |
| | | AGT TTT 3′ | | CGG ATG 3′ |
| ABCA6 | SEQ ID NO: 58 | 5′ AGT TGT GTT | SEQ ID NO: 59 | 5′ GTG CCT GAC |
| | | TTG TGC TGA | | TCT TTG GGT |
| | | GCC TCC 3′ | | GAC TTT 3′ |
| ABCA7 | SEQ ID NO: 60 | 5′ ATA GCA TGG | SEQ ID NO: 61 | 5′ TTT CAC CAC |
| | | AGG AGT GTG | | CAC GGC TTC |
| | | AAG CGC 3′ | | TCT CCA 3′ |
| ABCA8 | SEQ ID NO: 62 | 5′ GCT GGG | SEQ ID NO: 63 | 5′ GAA AAT GGC |
| | | TGA TTT TGA | | ACA CAG TTG |
| | | GGA GGA TTT 3′ | | GCT TAC 3′ |
| ABCA9 | SEQ ID NO: 64 | 5′ TGT GCC AGC | SEQ ID NO: 65 | 5′ TTT CTC CTA |
| | | AAC CAA ATC | | ATG CTA TCC |
| | | CCA TGT 3′ | | CTC CCC 3′ |
| ABCA10 | SEQ ID NO: 66 | 5′ AGG AGC | SEQ ID NO: 67 | 5′ GCC ATT TCA |
| | | TGG GAA ATG | | TCA GTT TAT |
| | | TTG ATG ATA 3′ | | CAG ACC 3′ |
| ABCA12 | SEQ ID NO: 68 | 5′ CCT GCT GGA | SEQ ID NO: 69 | 5′ ATG TTT GCG |
| | | GAG TGT TTT | | ACT CCT CCT |
| | | GGG CTT 3′ | | GCT GTG 3′ |
| ABCB1 | SEQ ID NO: 70 | 5′ CAT CCT GTT | SEQ ID NO: 71 | 5′ GCA AGG CAG |
| | | TGA CTG CAG | | TCA GTT ACA |
| | | CAT TGC 3′ | | GTC CAA 3′ |
| ABCB2 | SEQ ID NO: 72 | 5′ ATA TTG CCT | SEQ ID NO: 73 | 5′ TTC TCA GTT |
| | | ATG GCC TGA | | TCA GAG TGC |
| | | CCC AGA 3′ | | TGG CCA 3′ |
| ABCB3 | SEQ ID NO: 74 | 5′ GGG AGT | SEQ ID NO: 75 | 5′ TGC TCA TGG |
| | | AGG AGC TAT | | TCT AGT GGA |
| | | GCT AAG TGT 3′ | | AGG TCA 3′ |
| ABCB4 | SEQ ID NO: 76 | 5′ TTG ACA GCT | SEQ ID NO: 77 | 5′ CAT AAG TTC |
| | | ACA GTG AAG | | TGT GTC CCA |
| | | AGG GGC 3′ | | GCC TGG 3′ |
| ABCB6 | SEQ ID NO: 78 | 5′ TTC GGT TCT | SEQ ID NO: 79 | 5′ GAC CAG GAT |
| | | ACG ACA TCA | | GAA ATA AGC |
| | | GCT CTG 3′ | | CAG GGA 3′ |
| ABCB7 | SEQ ID NO: 80 | 5′ CCC TGC | SEQ ID NO: 81 | 5′ CTT AGC ACG |
| | | AGG AAA GAA | | AAC AGT TTC |
| | | AGT GGC CAT 3′ | | CAC AGC 3′ |
| ABCB8 | SEQ ID NO: 82 | 5′ AGG TTG TCG | SEQ ID NO: 83 | 5′ TTT ATT GTG |
| | | GTT TCA TCA | | AGC AGG AGC |
| | | GCC AGG 3′ | | AGC CGC 3′ |
| ABCB9 | SEQ ID NO: 84 | 5′ TGG ATC ACC | SEQ ID NO: 85 | 5′ TGC CAC CAT |
| | | GCT TCC TGC | | CCC ATC CAC |
| | | ATC TTG 3′ | | CAA AGA 3′ |
| ABCB10 | SEQ ID NO: 86 | 5′ GCA AGG | SEQ ID NO: 87 | 5′ GGT TTC TTC |
| | | CAT GAA CTG | | TTC CAG TCT |
| | | CTA GGT ATT 3′ | | AAT CAG 3′ |
| ABCB11 | SEQ ID NO: 88 | 5′ TTG TCA TTG | SEQ ID NO: 89 | 5′ AGA GCA TCC |
| | | CCC ATC GCT | | ACC CTT TCC |
| | | TGT CCA 3′ | | CTA TCC 3′ |
| ABCC1 | SEQ ID NO: 90 | 5′ GCT CCC ATC | SEQ ID NO: 91 | 5′ TGA GCA GGT |
| | | ACC TCT AAC | | ACC ATG AGA |
| | | ATC CTT 3′ | | GGG AAA 3′ |
| ABCC2 | SEQ ID NO: 92 | 5′ GTA GCA | SEQ ID NO: 93 | 5′ GGG TAG TAG |
| | | TGG AGA AGA | | GTT CAT GGG |
| | | TTG GTG TGG 3′ | | TGT TCA 3′ |
| ABCC3 | SEQ ID NO: 94 | 5′ CAA GAG | SEQ ID NO: 95 | 5′ TTT AAT GGA |
| | | CCG CAT CCT | | TTC AGG CAG |
| | | GGT TTT AGA 3′ | | CAC CCC 3′ |
| ABCC4 | SEQ ID NO: 96 | 5′ TGG GAA | SEQ ID NO: 97 | 5′ AAT GCC TTC |
| | | GAA CCG GAG | | GGA ACG GAC |
| | | CTG GAA AAA 3′ | | TTG ACA 3′ |
| ABCC5 | SEQ ID NO: 98 | 5′ AAG GAA | SEQ ID NO: 99 | 5′ AAA CCA CAC |
| | | GAC GTG TGG | | AGC AAC CAG |
| | | CAA TAG TGG 3′ | | CAA CCT 3′ |
| ABCC6 | SEQ ID NO: 100 | 5′ TCG TGT CAG | SEQ ID NO: 101 | 5′ CTG CCA CCT |
| | | TGG AGC GGA | | GCC CCT TGT |
| | | TGC AGG 3′ | | CCA TGA 3′ |
| ABCC7 | SEQ ID NO: 102 | 5′ TCT TTC ACA | SEQ ID NO: 103 | 5′ CAG TTT GGA |
| | | GGG GAC AGG | | GTT GAG AAG |
| | | ATG GTT 3′ | | GCA GTG 3′ |
| ABCC8 | SEQ ID NO: 104 | 5′ AAA CCG | SEQ ID NO: 105 | 5′ TGG GCT CTG |
| | | AGG CAG AGA | | GCA GGT CAC |
| | | GCT ACG AGG | | TTG TCT 3′ |
| | | 3′ | | |
| ABCC9 | SEQ ID NO: 106 | 5′ TGG GTG | SEQ ID NO: 107 | 5′ GTG GGC GAA |
| | | CAG TGA AGA | | CAA ATT TGG |
| | | AGG TGA ACA 3′ | | GAC AGT 3′ |
| ABCC10b | SEQ ID NO: 108 | 5′ TCT TCC CTG | SEQ ID NO: 109 | 5′ TGA AAA TGC |
| | | TTG TTG GTG | | AAG TGG GCT |
| | | CTC TTC 3′ | | CCT ATG 3′ |
| ABCC11 | SEQ ID NO: 110 | 5′ GAT TCT CAT | SEQ ID NO: 111 | 5′ TGG TTC TGG |
| | | TGA CGG CGT | | GGT TCT AAG |
| | | GGA CAT 3′ | | GTC TTG 3′ |
| ABCC12a | SEQ ID NO: 112 | 5′ CTG GTT ATG | SEQ ID NO: 113 | 5′ TTG CAA GGC |
| | | GAA AAT GGG | | GAC ATT TCA |
| | | AAG GTG 3′ | | GGG TAA 3′ |
| ABCC13 | SEQ ID NO: 114 | 5′ GCA CCT | SEQ ID NO: 115 | 5′ TAA CAA ACA |
| | | GTG GGC CAT | | CAA GGA CTG |
| | | ACT AAA AGA 3′ | | CCA CCC 3′ |
| ABCD1 | SEQ ID NO: 116 | 5′ TTC CCT CCT | SEQ ID NO: 117 | 5′ TCT TTG GCA |
| | | CGT CAG TCT | | CTG AGC TGG |
| | | CTC AAA 3′ | | GAA CAT 3′ |
| ABCD2 | SEQ ID NO: 118 | 5′ GTG GCC | SEQ ID NO: 119 | 5′ ACA AAA GAG |
| | | AAC TAA ACC | | CAC TAA ACC |
| | | TGT ACA AAA 3′ | | AGA GAG 3′ |
| ABCD3 | SEQ ID NO: 120 | 5′ TAC TCA TTC | SEQ ID NO: 121 | 5′ CTT CGG TAG |
| | | CTT GTG TGT | | CCA GTG ATT |
| | | GTC TTG 3′ | | GTT ATA 3′ |
| ABCD4 | SEQ ID NO: 122 | 5′ CTC CAT ATG | SEQ ID NO: 123 | 5′ AGA AGC CTG |
| | | CTT GAA GTG | | GCA AAC ATT |
| | | CTG ATT 3′ | | ATG AAG 3′ |
| ABCE1 | SEQ ID NO: 124 | 5′ ATT CCC CGC | SEQ ID NO: 125 | 5′ TGG GAG GGT |
| | | AAA AAA CCC | | AAT AAA GGG |
| | | CTA ACT 3′ | | AGA TCA 3′ |
| ABCF1 | SEQ ID NO: 126 | 5′ TTG GAG | SEQ ID NO: 127 | 5′ TTT CCT GCC |
| | | GCC CTG GGT | | CCA AGT CCT |
| | | GAA GTC ATG 3′ | | CAA CCA 3′ |
| ABCF2 | SEQ ID NO: 128 | 5′ TGC TAC CCA | SEQ ID NO: 129 | 5′ ACT TGG AGC |
| | | GAG ATC AAG | | TGG TGT ACT |
| | | GAG AAG 3′ | | TGG TGA 3′ |
| ABCF3 | SEQ ID NO: 130 | 5′ CCT AAA CGT | SEQ ID NO: 131 | 5′ TTT ACA TAG |
| | | CAG TGC TTG | | CAG CCA CTT |
| | | TGG AAC 3′ | | GGG GTC 3′ |
| ABCG1 | SEQ ID NO: 132 | 5′ CGT CTA GAA | SEQ ID NO: 133 | 5′ CCA GCT GGG |
| | | TCG AGG AGG | | TGA CTC GGG |
| | | CAA GCC 3′ | | TTA AAC 3′ |
| ABCG2 | SEQ ID NO: 134 | 5′ CAG TAC TTC | SEQ ID NO: 135 | 5′ GGG CTA CTA |
| | | AGC ATT CCA | | ACC TAC CTA |
| | | CGA TAT 3′ | | TTC ATT 3′ |
| ABCG4 | SEQ ID NO: 136 | 5′ ACA GGC ACA | SEQ ID NO: 137 | 5′ CAG GGA TGT |
| | | TAC ATG AGA | | GTA CAG GAA |
| | | ACA GGC 3′ | | AAA GGG 3′ |
| ABCG5 | SEQ ID NO: 138 | 5′ GCC CAG | SEQ ID NO: 139 | 5′ CCC TCG TGT |
| | | GTG CAA CAT | | GGA CAT CTG |
| | | CTA GAT TCA 3′ | | CAT TTA 3′ |
| ABCG8 | SEQ ID NO: 140 | 5′ TCA ATG ACC | SEQ ID NO: 141 | 5′ ACG TAG TAC |
| | | ATC GGC TTC | | AGG ACC ATG |
| | | CTC TAT 3′ | | AAG CCA 3′ |

TABLE 2

| | bt20 | caco2 | caov | colo320 | hbt161 | hek | hek2 | hepG2 |
|---|---|---|---|---|---|---|---|---|
| wt1 | 1.760147 | 1.618089 | 1.424148 | 1.161262 | 1.543551 | 1.987004 | 1.269118 | 1.040183 |
| abcA1 | 3.004793 | 3.347099 | 0.828726 | 2.370672 | 5.264526 | 3.609649 | 2.229297 | 1.564597 |
| abcA2 | 4.624323 | 5.260656 | 2.909857 | 4.169548 | 7.839229 | 6.17035 | 4.606237 | 2.813327 |
| sh1 | 3.911999 | 4.197328 | 1.598497 | 3.81232 | 6.301507 | 4.454382 | 4.764967 | 2.685198 |
| abcA3 | 7.608692 | 8.546286 | 4.317059 | 7.303269 | 10.71203 | 8.577419 | 6.242746 | 4.433605 |
| abcA4 | 5.919356 | 7.41871 | 4.700107 | 6.432769 | 7.527029 | 8.965953 | 7.075436 | 4.358784 |
| abcA5 | 2.957649 | 3.448256 | 0.89617 | 2.699665 | 4.770988 | 3.656885 | 4.230468 | 2.073816 |
| abcA6 | 5.118411 | 5.992906 | 2.218216 | 6.488178 | 7.475392 | 7.827521 | 6.676626 | 3.399797 |
| abcA7 | 0.813024 | 1.451727 | 0.423628 | 0.756805 | 0.925006 | 1.414885 | 1.045833 | 1.075277 |
| abcA8 | 3.766634 | 4.410631 | 1.384986 | 4.646162 | 5.742969 | 4.997531 | 3.498515 | 2.963095 |
| abcA9 | 6.745635 | 7.854984 | 2.973699 | 10.55948 | 11.38119 | 10.56285 | 10.78415 | 6.514659 |
| abcA10 | 3.731862 | 4.922754 | 0.973826 | 5.048334 | 7.438836 | 4.674079 | 5.928152 | 2.811801 |
| abcA12 | 4.602116 | 6.046777 | 2.419136 | 6.102304 | 6.873125 | 6.533879 | 5.779279 | 3.000348 |
| abcB1 | 6.22968 | 6.922212 | 3.192804 | 6.876342 | 8.412035 | 7.777775 | 6.954351 | 2.889171 |
| abcB2 | 4.692122 | 5.67407 | 2.941921 | 4.82886 | 7.075056 | 6.906656 | 5.510521 | 3.107792 |
| actin | 2.813648 | 2.554335 | 2.130733 | 2.901062 | 2.465122 | 3.19905 | 3.166989 | 1.849321 |
| abcB3 | 5.815124 | 6.389326 | 4.205251 | 6.253255 | 8.191762 | 8.021734 | 8.274497 | 3.410876 |
| abcB4 | 7.6835 | 8.38654 | 7.058921 | 10.66992 | 10.24258 | 12.10061 | 12.24046 | 6.266877 |
| abcB6 | 4.084981 | 4.984172 | 2.846189 | 4.31622 | 5.588544 | 5.753428 | 5.929129 | 2.891158 |
| abcB7 | 0.074123 | 0.157225 | 0.018996 | 0.219296 | 0.1168 | 0.212074 | 0.239904 | 0.212343 |
| abcB8 | 6.664505 | 7.71405 | 9.678823 | 7.429662 | 11.50949 | 8.885061 | 9.780714 | 6.916769 |
| abcB9 | 6.842912 | 7.795226 | 4.86316 | 8.057427 | 8.533891 | 9.765656 | 10.25564 | 3.976807 |
| abcB10 | 2.825061 | 3.106933 | 1.136532 | 2.301583 | 4.752371 | 3.201264 | 3.287397 | 2.280606 |
| abcB11 | 4.92933 | 5.926065 | 2.530428 | 6.016988 | 8.145644 | 7.257995 | 6.36416 | 3.383772 |
| abcC1 | 6.359446 | 8.127404 | 3.298729 | 7.453735 | 10.77491 | 8.893629 | 8.964621 | 4.780864 |
| abcC2 | 0.346905 | 0.577156 | 0.270287 | 0.384025 | 0.448287 | 0.650925 | 0.459208 | 0.268697 |
| abcC3 | 6.013665 | 7.439689 | 5.945232 | 6.209607 | 8.332764 | 9.269004 | 6.845003 | 4.983321 |
| tubulin | 2.298514 | 2.087163 | 1.772524 | 2.349229 | 2.136879 | 2.939272 | 2.398394 | 1.276152 |
| abcC4 | 3.220286 | 3.368696 | 1.862558 | 2.59325 | 4.503437 | 3.730238 | 3.052793 | 2.10763 |
| abcC5 | 7.291887 | 8.497259 | 7.300674 | 8.495691 | 8.914714 | 10.28172 | 9.36289 | 4.897317 |
| abcC6 | 5.308255 | 6.250884 | 5.862711 | 5.894473 | 6.01132 | 7.891434 | 6.363759 | 3.901863 |
| abcC7 | 6.225003 | 6.01575 | 6.515959 | 7.435329 | 7.629163 | 8.378556 | 9.526473 | 3.635269 |
| abcC8 | 0.20927 | 0.312892 | 0.104774 | 0.399283 | 0.316207 | 0.492878 | 0.391981 | 0.090541 |
| abcC9 | 4.64518 | 5.215398 | 3.603154 | 4.558374 | 6.240997 | 6.430868 | 5.816828 | 3.462583 |
| abcC10 | 4.421718 | 4.946461 | 4.121841 | 4.067659 | 5.83334 | 5.416718 | 5.460851 | 3.449476 |
| abcC11 | 5.377921 | 6.014355 | 4.830902 | 6.52645 | 7.497958 | 7.718198 | 7.957897 | 3.78826 |
| abcC12 | 4.350023 | 5.097199 | 2.083206 | 5.287654 | 6.740772 | 5.947075 | 5.545201 | 2.982741 |
| abcC13 | 3.289754 | 3.800043 | 1.178961 | 3.625486 | 6.344843 | 4.852034 | 4.386753 | 2.696062 |
| abcD1 | 3.324885 | 3.888023 | 1.527821 | 4.02903 | 4.896376 | 5.135681 | 5.520253 | 2.482413 |
| abcD2 | 2.691791 | 2.953712 | 0.973682 | 2.255953 | 5.322177 | 3.067491 | 3.760776 | 1.751205 |
| abcD3 | 2.140153 | 2.205829 | 0.720351 | 1.921719 | 3.083791 | 2.584265 | 2.338868 | 1.419681 |
| abcD4 | 4.14984 | 3.88578 | 1.643377 | 4.797625 | 5.881954 | 5.275499 | 5.142964 | 2.056191 |
| abcE1 | 3.861133 | 4.032411 | 1.123749 | 3.908748 | 5.426583 | 4.427617 | 4.13424 | 2.162943 |
| abcF1 | 3.006927 | 3.174756 | 1.434718 | 2.585275 | 4.162581 | 3.438596 | 3.736274 | 2.009307 |
| abcF2 | 4.790131 | 5.441651 | 3.842271 | 4.674228 | 5.87547 | 6.234563 | 5.246463 | 3.260485 |
| abcF3 | 4.204277 | 4.031992 | 3.819556 | 4.154403 | 6.220227 | 4.68503 | 4.375861 | 3.108019 |
| abcG1 | 4.681105 | 5.047226 | 4.150557 | 4.956645 | 6.502208 | 6.122183 | 5.994359 | 3.498153 |
| abcG2 | 3.651149 | 3.601846 | 1.405222 | 3.235066 | 6.296545 | 4.418258 | 3.541676 | 2.341017 |
| abcG4 | 5.790807 | 7.229536 | 3.666519 | 7.763176 | 9.084905 | 8.305757 | 8.253085 | 3.405108 |
| abcG5 | 1.849521 | 2.966887 | 0.447623 | 1.853678 | 4.376194 | 2.754429 | 2.842199 | 1.776374 |
| abcG8 | 3.423202 | 4.417621 | 1.874501 | 4.016039 | 5.100572 | 4.995688 | 4.445849 | 2.438934 |
| | ht75 | ht177 | lncap | mcf7 | mda453 | mda468 | mfe29c | skmes1 |
| wt1 | 1.176464 | 1.602791 | 1.511799 | 1.655671 | 1.172517 | 1.364591 | 1.257281 | 2.173767 |
| abcA1 | 2.908955 | 4.401204 | 5.121514 | 3.141233 | 1.70476 | 3.274642 | 3.10138 | 2.974131 |
| abcA2 | 3.969959 | 6.672095 | 6.680252 | 5.119029 | 3.48243 | 4.697005 | 4.960736 | 4.745097 |
| sh1 | 3.817439 | 5.271368 | 6.99939 | 4.016223 | 2.307139 | 4.61252 | 3.669887 | 3.949323 |
| abcA3 | 6.165651 | 12.94566 | 11.60684 | 7.93316 | 5.148105 | 6.795211 | 7.890175 | 7.28488 |
| abcA4 | 4.721274 | 8.165924 | 8.144223 | 6.643413 | 4.827968 | 5.660874 | 7.521435 | 7.213923 |
| abcA5 | 4.231976 | 3.708588 | 5.477282 | 3.097772 | 1.716878 | 4.339422 | 2.885842 | 3.511156 |
| abcA6 | 4.716604 | 7.380911 | 8.603165 | 5.66999 | 4.405 | 5.3359 | 5.705216 | 6.032574 |
| abcA7 | 3.307867 | 1.28646 | 1.089256 | 0.780969 | 0.415919 | 1.64963 | 0.951143 | 1.189434 |
| abcA8 | 3.956494 | 5.261313 | 6.470901 | 4.355213 | 2.630278 | 4.403083 | 3.568671 | 4.097393 |
| abcA9 | 6.197568 | 12.38926 | 13.07479 | 7.775826 | 6.282562 | 7.08791 | 8.729474 | 6.184259 |
| abcA10 | 3.495781 | 8.054075 | 8.295051 | 3.573241 | 2.815333 | 4.373692 | 4.944166 | 2.483748 |
| abcA12 | 3.891084 | 7.761779 | 7.458326 | 5.199048 | 3.419758 | 4.652229 | 5.750435 | 4.411436 |
| abcB1 | 4.556779 | 9.130438 | 9.47086 | 6.403695 | 4.676444 | 5.728989 | 6.192011 | 6.088017 |
| abcB2 | 4.274465 | 7.468082 | 6.484062 | 5.198367 | 3.292553 | 5.056685 | 4.284439 | 4.855056 |
| actin | 2.14277 | 4.099607 | 2.0519 | 2.663159 | 2.310214 | 2.052918 | 2.548381 | 2.572973 |
| abcB3 | 5.028789 | 8.114998 | 7.876121 | 6.406229 | 4.147808 | 6.190622 | 4.899937 | 6.742927 |
| abcB4 | 6.656844 | 12.67506 | 11.05904 | 8.330893 | 6.309777 | 7.778177 | 7.964991 | 7.427915 |
| abcB6 | 4.190264 | 6.217372 | 5.877825 | 4.392595 | 2.776184 | 4.565543 | 4.09943 | 4.31774 |
| abcB7 | 0.734458 | 0.136103 | 0.192291 | 0.110247 | 0.109597 | 0.281218 | 0.195551 | 0.104149 |
| abcB8 | 6.525109 | 10.77551 | 11.0921 | 8.146399 | 5.277253 | 7.358466 | 6.259928 | 7.008684 |
| abcB9 | 5.274958 | 11.30368 | 9.473696 | 7.5665 | 5.272494 | 6.691976 | 7.747736 | 6.527339 |
| abcB10 | 3.301957 | 3.55259 | 4.940379 | 2.939483 | 1.635918 | 4.142617 | 2.380918 | 3.384194 |
| abcB11 | 4.245162 | 10.16901 | 8.433979 | 4.948483 | 3.954381 | 5.200344 | 6.509304 | 4.331175 |
| abcC1 | 5.50021 | 13.61978 | 12.0042 | 6.806182 | 4.932777 | 6.809653 | 8.57131 | 5.022411 |
| abcC2 | 1.332171 | 0.572141 | 0.508614 | 0.350252 | 0.256199 | 0.385886 | 0.326415 | 0.386984 |
| abcC3 | 4.872408 | 9.18837 | 8.199831 | 6.753851 | 5.106647 | 5.919573 | 6.765984 | 5.417606 |
| tubulin | 1.406254 | 3.644242 | 2.058773 | 2.057926 | 1.787842 | 1.659775 | 2.06705 | 2.045376 |
| abcC4 | 3.121299 | 4.304639 | 5.198264 | 3.352392 | 2.042043 | 4.142815 | 2.622131 | 3.34671 |
| abcC5 | 6.726237 | 12.91419 | 9.846679 | 7.445036 | 5.63811 | 7.616253 | 8.361338 | 7.356883 |
| abcC6 | 4.506416 | 8.766663 | 7.077777 | 5.604559 | 4.04964 | 5.496995 | 5.453474 | 5.151823 |
| abcC7 | 5.335326 | 9.472832 | 8.788647 | 7.166225 | 4.766767 | 6.51443 | 6.271785 | 6.575034 |
| abcC8 | 0.810137 | 0.344879 | 0.512981 | 0.254042 | 0.166017 | 0.493377 | 0.347002 | 0.266 |
| abcC9 | 4.003031 | 7.085351 | 6.89536 | 5.163141 | 3.255498 | 5.129568 | 4.606734 | 4.810717 |
| abcC10 | 4.179319 | 6.005754 | 6.226844 | 4.169301 | 2.88363 | 4.755964 | 4.331723 | 4.556479 |
| abcC11 | 3.902178 | 8.996234 | 8.125819 | 6.068471 | 4.413025 | 5.404135 | 6.297336 | 5.365717 |
| abcC12 | 3.771503 | 8.863798 | 7.793826 | 4.225222 | 3.43731 | 5.073856 | 5.931341 | 3.568024 |
| abcC13 | 3.18993 | 5.974607 | 7.17708 | 2.957792 | 2.417687 | 3.972703 | 3.082481 | 1.722248 |
| abcD1 | 3.418455 | 5.989764 | 5.104864 | 3.723436 | 2.563685 | 4.31724 | 3.968746 | 3.12896 |
| abcD2 | 3.294883 | 3.966396 | 5.309153 | 2.865471 | 1.680424 | 4.019171 | 2.339185 | 3.28914 |
| abcD3 | 2.747711 | 2.203203 | 3.483447 | 2.123141 | 1.126501 | 3.274346 | 1.543806 | 2.419758 |
| abcD4 | 4.663416 | 3.004363 | 6.999542 | 4.275728 | 2.857245 | 5.54243 | 4.592518 | 4.056683 |
| abcE1 | 3.777744 | 3.46213 | 6.370534 | 3.811598 | 2.286778 | 4.590051 | 3.491907 | 3.120769 |
| abcF1 | 3.978386 | 3.141324 | 4.572986 | 2.925329 | 1.656583 | 4.641542 | 2.397631 | 3.24873 |
| abcF2 | 4.377511 | 3.836889 | 6.585178 | 4.966859 | 3.338441 | 5.314146 | 4.688036 | 4.673684 |
| abcF3 | 4.057537 | 4.08325 | 6.796362 | 4.424878 | 2.649993 | 5.247922 | 4.126422 | 4.173554 |
| abcG1 | 4.096599 | 4.883075 | 6.594222 | 4.918009 | 3.110898 | 5.169477 | 4.696716 | 4.616634 |
| abcG2 | 4.123002 | 3.674573 | 6.905581 | 4.337574 | 1.97057 | 5.424299 | 3.38139 | 4.576475 |
| abcG4 | 4.574626 | 7.435952 | 9.835062 | 7.730029 | 4.750124 | 6.335067 | 7.198438 | 4.975459 |
| abcG5 | 4.73147 | 3.294423 | 4.270417 | 2.125876 | 0.96839 | 2.808186 | 1.864413 | 1.149096 |
| abcG8 | 4.338727 | 4.759615 | 6.102491 | 3.924575 | 1.699189 | 5.148777 | 3.706877 | 3.627643 |

| | sknas | sknbe | sknd2 | sknmc | t47d | zr75 | mdck |
|---|---|---|---|---|---|---|---|
| wt1 | 0.626002 | 1.296755 | 1.626099 | 1.447571 | 1.850718 | 1.613761 | 1.412566 |
| abcA1 | 1.551803 | 2.454523 | 3.391316 | 2.096912 | 4.58395 | 2.551371 | 2.453901 |
| abcA2 | 1.381523 | 3.811958 | 5.770854 | 4.222122 | 6.998368 | 3.750876 | 3.993493 |
| sh1 | 1.082382 | 3.328175 | 4.988276 | 3.187279 | 5.914972 | 2.348161 | 4.96644 |
| abcA3 | 2.992319 | 6.880617 | 9.331218 | 6.042775 | 9.776199 | 5.348274 | 5.13229 |
| abcA4 | 1.237927 | 5.505667 | 7.847185 | 6.343008 | 8.173683 | 5.36549 | 5.248557 |
| abcA5 | 1.228945 | 2.558133 | 3.774496 | 2.166136 | 5.289771 | 2.315449 | 3.184366 |
| abcA6 | 1.482493 | 4.932076 | 6.965578 | 4.76329 | 7.771708 | 3.71513 | 4.6072 |
| abcA7 | 1.169245 | 0.678497 | 0.942581 | 0.687287 | 1.060294 | 0.627354 | 1.031147 |
| abcA8 | 1.461138 | 3.412184 | 4.423174 | 2.700437 | 6.046554 | 2.900982 | 3.329838 |
| abcA9 | 1.466374 | 8.409215 | 11.61034 | 6.872109 | 10.10557 | 4.556159 | 7.380374 |
| abcA10 | 0.745729 | 4.228018 | 4.854904 | 3.338708 | 6.187728 | 3.136154 | 5.292145 |
| abcA12 | 1.072138 | 4.430771 | 5.938261 | 4.437994 | 6.614576 | 4.085293 | 5.922869 |
| abcB1 | 1.692989 | 5.584571 | 8.058913 | 5.604806 | 8.288023 | 5.028871 | 6.296814 |
| abcB2 | 2.530281 | 3.636367 | 7.826283 | 3.515015 | 6.949947 | 5.583017 | 5.0383 |
| actin | 1.296333 | 2.524045 | 4.114827 | 2.527953 | 2.729459 | 1.244456 | 2.843669 |
| abcB3 | 2.140021 | 5.433807 | 9.335644 | 3.940163 | 8.353189 | 4.062514 | 7.246606 |
| abcB4 | 3.12579 | 8.712582 | 14.48269 | 7.130204 | 10.49239 | 5.045277 | 7.209005 |
| abcB6 | 1.576024 | 3.79744 | 6.837115 | 3.318637 | 6.187565 | 2.415473 | 5.259997 |
| abcB7 | 0.041012 | 0.174013 | 0.296479 | 0.234935 | 0.208883 | 0.055861 | 0.204321 |
| abcB8 | 1.860419 | 7.115313 | 11.42079 | 5.129343 | 9.811637 | 3.712953 | 9.161328 |
| abcB9 | 2.833434 | 6.032784 | 11.33989 | 5.270131 | 8.599939 | 3.976051 | 7.62494 |
| abcB10 | 0.973833 | 2.546677 | 3.908612 | 1.864896 | 4.447705 | 1.538376 | 3.513867 |
| abcB11 | 1.016901 | 5.457363 | 7.892148 | 4.303962 | 6.55196 | 2.457971 | 5.433564 |
| abcC1 | 1.160266 | 7.348775 | 9.434584 | 6.013765 | 8.637754 | 4.302263 | 9.586915 |
| abcC2 | 0.388017 | 0.422901 | 0.510708 | 0.291655 | 0.276086 | 0.118947 | 0.375365 |
| abcC3 | 1.678748 | 6.148746 | 8.915554 | 5.596885 | 7.878307 | 3.581028 | 6.422337 |
| tubulin | 1.004109 | 1.982293 | 2.084784 | 2.470466 | 2.11105 | 1.445386 | 2.455287 |
| abcC4 | 2.057933 | 2.277096 | 3.143111 | 2.232597 | 4.564203 | 2.913778 | 3.438326 |
| abcC5 | 4.697307 | 8.860292 | 10.07361 | 8.428701 | 10.03339 | 6.205289 | 9.101034 |
| abcC6 | 2.60295 | 4.688247 | 6.461338 | 4.944702 | 6.983595 | 4.43756 | 6.433565 |
| abcC7 | 3.144826 | 6.454929 | 8.549991 | 6.111629 | 8.626731 | 6.350593 | 8.475225 |
| abcC8 | 0.196555 | 0.365982 | 0.329052 | 0.330411 | 0.409944 | 0.03408 | 0.161494 |
| abcC9 | 1.717879 | 4.781281 | 6.920567 | 5.053801 | 6.745872 | 3.654303 | 5.490752 |
| abcC10 | 1.645101 | 4.423686 | 5.026144 | 4.751168 | 5.529159 | 4.168599 | 5.561204 |
| abcC11 | 1.602617 | 5.326979 | 7.10816 | 5.700667 | 7.456445 | 4.850691 | 7.009039 |
| abcC12 | 1.343014 | 4.877474 | 5.826324 | 4.41935 | 6.384966 | 4.666285 | 5.233725 |
| abcC13 | 0.80427 | 4.023579 | 3.746475 | 3.086382 | 4.387412 | 3.643549 | 4.041307 |
| abcD1 | 1.575417 | 4.202042 | 5.104484 | 3.949438 | 5.473419 | 3.450191 | 5.11301 |
| abcD2 | 1.101583 | 2.982265 | 3.360074 | 2.355337 | 4.726608 | 1.887609 | 3.95424 |
| abcD3 | 1.485437 | 2.021632 | 2.86093 | 1.343676 | 3.745222 | 1.837798 | 2.626086 |
| abcD4 | 2.764205 | 4.550051 | 6.468077 | 3.853675 | 6.30511 | 3.196361 | 4.226271 |
| abcE1 | 1.952447 | 3.538173 | 4.758896 | 2.653818 | 5.231657 | 3.201301 | 3.925311 |
| abcF1 | 1.802607 | 3.19475 | 4.550298 | 2.413411 | 5.138317 | 2.691841 | 4.668573 |
| abcF2 | 2.540565 | 3.953334 | 6.320643 | 4.08181 | 6.460594 | 3.856447 | 6.072975 |
| abcF3 | 2.113369 | 2.789391 | 5.566595 | 3.58488 | 5.853848 | 3.048415 | 4.769793 |
| abcG1 | 2.266889 | 1.886996 | 6.717191 | 4.143091 | 6.73207 | 3.486553 | 6.323139 |
| abcG2 | 1.676385 | 1.62062 | 5.419817 | 2.920977 | 5.7438 | 2.41731 | 3.931884 |
| abcG4 | 1.482581 | 1.996986 | 8.376657 | 5.91003 | 7.746507 | 5.923334 | 8.298024 |
| abcG5 | 0.533571 | 0.697843 | 2.381646 | 1.480494 | 2.896487 | 1.586755 | 3.564191 |
| abcG8 | 1.124542 | 0.889562 | 6.172538 | 3.63882 | 6.247417 | 3.856546 | 5.077863 |

TABLE 3

| | bt20 | caco2 | caov | colo320 | hbt161 | hek | hek2 | hepG2 |
|---|---|---|---|---|---|---|---|---|
| wt1 | 0.625575 | 0.633468 | 0.668384 | 0.400289 | 0.626156 | 0.621123 | 0.400733 | 0.562468 |
| abcA1 | 1.067935 | 1.31036 | 0.38894 | 0.817174 | 2.135605 | 1.12835 | 0.703917 | 0.846039 |
| abcA2 | 1.643533 | 2.059501 | 1.36566 | 1.437249 | 3.180057 | 1.928807 | 1.454453 | 1.521275 |
| sh1 | 1.390365 | 1.643217 | 0.75021 | 1.314112 | 2.556266 | 1.392408 | 1.504573 | 1.451991 |
| abcA3 | 2.704209 | 3.345796 | 2.026091 | 2.517446 | 4.345434 | 2.68124 | 1.971193 | 2.397423 |
| abcA4 | 2.103801 | 2.90436 | 2.205864 | 2.217384 | 3.053411 | 2.802692 | 2.23412 | 2.356964 |
| abcA5 | 1.051179 | 1.349962 | 0.420592 | 0.930578 | 1.935396 | 1.143116 | 1.335801 | 1.121393 |
| abcA6 | 1.819137 | 2.34617 | 1.041058 | 2.236483 | 3.032463 | 2.446827 | 2.108193 | 1.838403 |
| abcA7 | 0.288957 | 0.568339 | 0.198818 | 0.260872 | 0.375237 | 0.442283 | 0.330229 | 0.581444 |
| abcA8 | 1.338701 | 1.726724 | 0.650005 | 1.601538 | 2.32969 | 1.562192 | 1.104682 | 1.602261 |
| abcA9 | 2.397469 | 3.075158 | 1.395623 | 3.639867 | 4.616888 | 3.301872 | 3.405173 | 3.52273 |
| abcA10 | 1.326343 | 1.927215 | 0.457038 | 1.740167 | 3.017634 | 1.461084 | 1.871857 | 1.52045 |
| abcA12 | 1.63564 | 2.36726 | 1.135354 | 2.103472 | 2.788148 | 2.042444 | 1.82485 | 1.622405 |
| abcB1 | 2.214094 | 2.709986 | 1.498453 | 2.370284 | 3.412421 | 2.431277 | 2.195887 | 1.562287 |
| abcB2 | 1.667629 | 2.221349 | 1.380709 | 1.664514 | 2.870063 | 2.158971 | 1.739987 | 1.680504 |
| abcB3 | 2.066756 | 2.501365 | 1.973617 | 2.155505 | 3.323066 | 2.507536 | 2.612733 | 1.844393 |
| abcB4 | 2.730797 | 3.283257 | 3.312908 | 3.677934 | 4.154998 | 3.782563 | 3.865015 | 3.388745 |
| abcB6 | 1.451845 | 1.95126 | 1.335779 | 1.487807 | 2.267046 | 1.79848 | 1.872166 | 1.563362 |
| abcB7 | 0.026344 | 0.061552 | 0.008915 | 0.075592 | 0.047381 | 0.066293 | 0.075751 | 0.114822 |
| abcB8 | 2.368635 | 3.019983 | 4.542486 | 2.561014 | 4.668934 | 2.777406 | 3.088332 | 3.740167 |
| abcB9 | 2.432043 | 3.051763 | 2.282388 | 2.777405 | 3.461854 | 3.052674 | 3.238294 | 2.150415 |
| abcB10 | 1.004056 | 1.216337 | 0.5334 | 0.793359 | 1.927844 | 1.000692 | 1.038019 | 1.233213 |
| abcB11 | 1.751936 | 2.320003 | 1.187586 | 2.074063 | 3.304357 | 2.268797 | 2.00953 | 1.829737 |
| abcC1 | 2.260214 | 3.181808 | 1.548167 | 2.569312 | 4.370943 | 2.780085 | 2.830645 | 2.585199 |
| abcC2 | 0.123294 | 0.225951 | 0.126852 | 0.132374 | 0.181852 | 0.203474 | 0.144998 | 0.145295 |
| abcC3 | 2.13732 | 2.912573 | 2.790229 | 2.14046 | 3.380264 | 2.897424 | 2.16136 | 2.694676 |
| tubulin | 0.816916 | 0.817106 | 0.831885 | 0.809782 | 0.866845 | 0.918795 | 0.75731 | 0.690065 |
| abcC4 | 1.144524 | 1.318815 | 0.87414 | 0.893897 | 1.826862 | 1.166046 | 0.963942 | 1.139678 |
| abcC5 | 2.591613 | 3.326603 | 3.426368 | 2.928476 | 3.616338 | 3.213992 | 2.956401 | 2.64817 |
| abcC6 | 1.88661 | 2.447166 | 2.7515 | 2.031832 | 2.438549 | 2.466806 | 2.009403 | 2.10989 |
| abcC7 | 2.212431 | 2.355114 | 3.058084 | 2.562967 | 3.094842 | 2.619076 | 3.008053 | 1.965731 |
| abcC8 | 0.074377 | 0.122495 | 0.049173 | 0.137633 | 0.128273 | 0.15407 | 0.123771 | 0.048959 |
| abcC9 | 1.650946 | 2.041783 | 1.69104 | 1.571277 | 2.531719 | 2.010243 | 1.836706 | 1.872353 |
| abcC10 | 1.571525 | 1.936496 | 1.934471 | 1.402127 | 2.366349 | 1.693227 | 1.724303 | 1.865266 |
| abcC11 | 1.91137 | 2.354568 | 2.267249 | 2.249676 | 3.041617 | 2.412653 | 2.512764 | 2.04846 |
| abcC12 | 1.546044 | 1.995509 | 0.977695 | 1.822661 | 2.734458 | 1.859013 | 1.750938 | 1.612884 |
| abcC13 | 1.169213 | 1.487684 | 0.553312 | 1.24971 | 2.573845 | 1.516711 | 1.385149 | 1.457866 |
| abcD1 | 1.181699 | 1.522127 | 0.71704 | 1.388812 | 1.986261 | 1.605377 | 1.74306 | 1.342337 |
| abcD2 | 0.956691 | 1.156353 | 0.45697 | 0.77763 | 2.158991 | 0.958876 | 1.187492 | 0.946945 |
| abcD3 | 0.760633 | 0.863563 | 0.338077 | 0.662419 | 1.250969 | 0.807823 | 0.738515 | 0.767677 |
| gdh | 0.355411 | 0.391491 | 0.469322 | 0.344701 | 0.405659 | 0.312593 | 0.315757 | 0.540739 |
| abcD4 | 1.474897 | 1.521249 | 0.771273 | 1.653748 | 2.38607 | 1.649083 | 1.623929 | 1.111863 |
| abcE1 | 1.372287 | 1.578654 | 0.5274 | 1.34735 | 2.201345 | 1.384041 | 1.305416 | 1.169587 |
| abcF1 | 1.068693 | 1.242889 | 0.673345 | 0.891148 | 1.68859 | 1.07488 | 1.179756 | 1.086511 |
| abcF2 | 1.702463 | 2.130359 | 1.803263 | 1.611212 | 2.38344 | 1.94888 | 1.656609 | 1.763072 |
| abcF3 | 1.494244 | 1.57849 | 1.792602 | 1.432028 | 2.523294 | 1.464507 | 1.38171 | 1.680627 |
| abcG1 | 1.663714 | 1.975945 | 1.947948 | 1.708562 | 2.637682 | 1.913751 | 1.892763 | 1.891588 |
| abcG2 | 1.297657 | 1.410091 | 0.659502 | 1.115132 | 2.554253 | 1.381116 | 1.11831 | 1.265879 |
| abcG4 | 2.058114 | 2.8303 | 1.720778 | 2.675977 | 3.685378 | 2.59632 | 2.605972 | 1.841274 |
| abcG5 | 0.657339 | 1.16151 | 0.210079 | 0.638965 | 1.775244 | 0.861015 | 0.897445 | 0.960555 |
| abcG8 | 1.216642 | 1.72946 | 0.879745 | 1.384334 | 2.069095 | 1.561616 | 1.403809 | 1.318827 |
| | ht75 | ht177 | lncap | mcf7 | mda453 | mda468 | mfe29c | skmes1 |
| wt1 | 0.549039 | 0.390962 | 0.73678 | 0.621694 | 0.507536 | 0.664708 | 0.493364 | 0.844846 |
| abcA1 | 1.357568 | 1.073567 | 2.495985 | 1.179514 | 0.737923 | 1.595116 | 1.217 | 1.155912 |
| abcA2 | 1.852723 | 1.627496 | 3.255641 | 1.922165 | 1.507406 | 2.287965 | 1.946622 | 1.844208 |
| sh1 | 1.781544 | 1.285823 | 3.411175 | 1.508067 | 0.998669 | 2.246812 | 1.440085 | 1.534926 |
| abcA3 | 2.877421 | 3.157781 | 5.656632 | 2.978854 | 2.228411 | 3.310026 | 3.096152 | 2.831308 |
| abcA4 | 2.203351 | 1.99188 | 3.969112 | 2.494561 | 2.089836 | 2.757477 | 2.951456 | 2.80373 |
| abcA5 | 1.975003 | 0.90462 | 2.66937 | 1.163195 | 0.743168 | 2.113783 | 1.132422 | 1.36463 |
| abcA6 | 2.201172 | 1.800395 | 4.192779 | 2.129047 | 1.90675 | 2.599178 | 2.238761 | 2.344593 |
| abcA7 | 1.543734 | 0.313801 | 0.530852 | 0.293249 | 0.180035 | 0.803554 | 0.373234 | 0.46228 |
| abcA8 | 1.846439 | 1.28337 | 3.153613 | 1.635356 | 1.138543 | 2.144792 | 1.400368 | 1.592474 |
| abcA9 | 2.892316 | 3.02206 | 6.372039 | 2.919776 | 2.719472 | 3.452603 | 3.425497 | 2.403546 |
| abcA10 | 1.631431 | 1.964597 | 4.042618 | 1.34173 | 1.218646 | 2.130476 | 1.94012 | 0.965322 |
| abcA12 | 1.815913 | 1.893298 | 3.634838 | 1.952211 | 1.480277 | 2.266154 | 2.256505 | 1.714529 |
| abcB1 | 2.126583 | 2.227149 | 4.615653 | 2.404548 | 2.024247 | 2.790657 | 2.429782 | 2.366141 |
| abcB2 | 1.994832 | 1.821658 | 3.160028 | 1.951955 | 1.425216 | 2.46317 | 1.681239 | 1.886944 |
| abcB3 | 2.346864 | 1.979457 | 3.838452 | 2.4055 | 1.795421 | 3.015524 | 1.922764 | 2.620675 |
| abcB4 | 3.106654 | 3.091775 | 5.389659 | 3.1282 | 2.731252 | 3.78884 | 3.12551 | 2.8869 |
| abcB6 | 1.955536 | 1.516577 | 2.864576 | 1.649393 | 1.2017 | 2.223929 | 1.608641 | 1.678113 |
| abcB7 | 0.342761 | 0.033199 | 0.093714 | 0.041397 | 0.04744 | 0.136985 | 0.076735 | 0.040478 |
| abcB8 | 3.045175 | 2.628425 | 5.405767 | 3.058923 | 2.284313 | 3.584394 | 2.456433 | 2.723963 |
| abcB9 | 2.461747 | 2.757258 | 4.617035 | 2.841175 | 2.282253 | 3.259739 | 3.040258 | 2.536886 |
| abcB10 | 1.540976 | 0.866568 | 2.407709 | 1.103758 | 0.708124 | 2.017916 | 0.934286 | 1.315285 |
| abcB11 | 1.981156 | 2.480483 | 4.110326 | 1.858125 | 1.711694 | 2.533148 | 2.554289 | 1.683335 |
| abcC1 | 2.566869 | 3.322217 | 5.850286 | 2.55568 | 2.135203 | 3.317061 | 3.363433 | 1.951987 |
| abcC2 | 0.621705 | 0.13956 | 0.247874 | 0.131517 | 0.110898 | 0.18797 | 0.128087 | 0.150404 |
| abcC3 | 2.273883 | 2.241281 | 3.996213 | 2.53603 | 2.210465 | 2.883492 | 2.655012 | 2.105582 |
| tubulin | 0.656278 | 0.888925 | 1.003349 | 0.772739 | 0.773886 | 0.808495 | 0.811123 | 0.794946 |
| abcC4 | 1.456665 | 1.050013 | 2.53339 | 1.258803 | 0.883919 | 2.018013 | 1.02894 | 1.300717 |
| abcC5 | 3.139039 | 3.150105 | 4.798809 | 2.795566 | 2.440514 | 3.709965 | 3.281039 | 2.859293 |
| abcC6 | 2.10308 | 2.138415 | 3.449377 | 2.104478 | 1.752928 | 2.67765 | 2.139976 | 2.002284 |
| abcC7 | 2.48992 | 2.310668 | 4.283174 | 2.690874 | 2.063344 | 3.173254 | 2.461086 | 2.555423 |
| abcC8 | 0.378079 | 0.084125 | 0.250003 | 0.095391 | 0.071862 | 0.240329 | 0.136166 | 0.103383 |
| abcC9 | 1.868157 | 1.7283 | 3.360475 | 1.938728 | 1.409176 | 2.498672 | 1.80771 | 1.869711 |
| abcC10 | 1.950428 | 1.464958 | 3.034672 | 1.565547 | 1.248209 | 2.316685 | 1.699794 | 1.7709 |
| abcC11 | 1.821091 | 2.194414 | 3.960143 | 2.278674 | 1.910223 | 2.632417 | 2.471112 | 2.085415 |
| abcC12 | 1.760107 | 2.162109 | 3.798345 | 1.586545 | 1.487875 | 2.471534 | 2.327494 | 1.386732 |
| abcC13 | 1.488695 | 1.457361 | 3.497772 | 1.110633 | 1.046521 | 1.93515 | 1.209584 | 0.669361 |
| abcD1 | 1.595344 | 1.461058 | 2.487871 | 1.398128 | 1.109717 | 2.102977 | 1.557359 | 1.216087 |
| abcD2 | 1.537675 | 0.967506 | 2.587432 | 1.075967 | 0.727389 | 1.957784 | 0.91791 | 1.278342 |
| abcD3 | 1.282318 | 0.537418 | 1.697669 | 0.797227 | 0.487618 | 1.594972 | 0.605799 | 0.940452 |
| gdh | 0.466686 | 0.243926 | 0.487353 | 0.375494 | 0.43286 | 0.487112 | 0.392406 | 0.388655 |
| abcD4 | 2.176349 | 0.732842 | 3.411248 | 1.60551 | 1.236788 | 2.699782 | 1.802131 | 1.576652 |
| abcE1 | 1.763019 | 0.844503 | 3.104699 | 1.431232 | 0.989855 | 2.235867 | 1.370245 | 1.212904 |
| abcF1 | 1.856656 | 0.76625 | 2.228659 | 1.098443 | 0.717069 | 2.260949 | 0.940845 | 1.262637 |
| abcF2 | 2.042922 | 0.935916 | 3.209307 | 1.865025 | 1.445079 | 2.588582 | 1.839613 | 1.816453 |
| abcF3 | 1.893594 | 0.99601 | 3.312228 | 1.661515 | 1.147077 | 2.556323 | 1.619232 | 1.622074 |
| abcG1 | 1.911824 | 1.191108 | 3.213714 | 1.846683 | 1.346584 | 2.518112 | 1.843019 | 1.79428 |
| abcG2 | 1.924146 | 0.896323 | 3.365456 | 1.628733 | 0.852981 | 2.642239 | 1.326877 | 1.778672 |
| abcG4 | 2.134913 | 1.813821 | 4.793148 | 2.902579 | 2.05614 | 3.085885 | 2.82471 | 1.933739 |
| abcG5 | 2.208109 | 0.803595 | 2.081201 | 0.798254 | 0.419177 | 1.3679 | 0.731607 | 0.446602 |
| abcG8 | 2.024822 | 1.160993 | 2.974068 | 1.473654 | 0.735512 | 2.508029 | 1.454601 | 1.409903 |

| | sknas | sknbe | sknd2 | sknmc | t47d | zr75 | mdck |
|---|---|---|---|---|---|---|---|
| wt1 | 0.482902 | 0.513761 | 0.39518 | 0.572626 | 0.678053 | 1.29676 | 0.496741 |
| abcA1 | 1.197071 | 0.972456 | 0.82417 | 0.82949 | 1.679435 | 2.050189 | 0.862935 |
| abcA2 | 1.065716 | 1.510258 | 1.402453 | 1.670174 | 2.564013 | 3.014068 | 1.404345 |
| sh1 | 0.834957 | 1.318588 | 1.212269 | 1.260814 | 2.167086 | 1.886898 | 1.74649 |
| abcA3 | 2.308296 | 2.726028 | 2.267706 | 2.390383 | 3.581735 | 4.297679 | 1.804813 |
| abcA4 | 0.954946 | 2.181287 | 1.907051 | 2.509148 | 2.994617 | 4.311513 | 1.845699 |
| abcA5 | 0.948017 | 1.013505 | 0.917291 | 0.856874 | 1.938029 | 1.860611 | 1.119809 |
| abcA6 | 1.143606 | 1.954037 | 1.6928 | 1.884248 | 2.847344 | 2.985344 | 1.62016 |
| abcA7 | 0.901964 | 0.268814 | 0.229069 | 0.271875 | 0.388463 | 0.504119 | 0.362611 |
| abcA8 | 1.127132 | 1.351872 | 1.074935 | 1.068231 | 2.215294 | 2.331124 | 1.170965 |
| abcA9 | 1.131171 | 3.331643 | 2.821586 | 2.718448 | 3.702407 | 3.661164 | 2.595371 |
| abcA10 | 0.575261 | 1.675096 | 1.179856 | 1.320716 | 2.267016 | 2.5201 | 1.861027 |
| abcA12 | 0.827054 | 1.755425 | 1.443137 | 1.755568 | 2.423402 | 3.282793 | 2.082827 |
| abcB1 | 1.305983 | 2.212548 | 1.958506 | 2.217132 | 3.036508 | 4.041019 | 2.214327 |
| abcB2 | 1.951877 | 1.44069 | 1.901971 | 1.390459 | 2.546273 | 4.48631 | 1.771761 |
| abcB3 | 1.650827 | 2.152817 | 2.268781 | 1.558638 | 3.060383 | 3.264489 | 2.54833 |
| abcB4 | 2.411256 | 3.451834 | 3.519634 | 2.820545 | 3.844128 | 4.054202 | 2.535107 |
| abcB6 | 1.215756 | 1.504506 | 1.66158 | 1.312776 | 2.266957 | 1.940987 | 1.849722 |
| abcB7 | 0.031637 | 0.068942 | 0.072051 | 0.092935 | 0.076529 | 0.044888 | 0.071851 |
| abcB8 | 1.43514 | 2.819012 | 2.77552 | 2.02905 | 3.594719 | 2.983594 | 3.221658 |
| abcB9 | 2.185731 | 2.390126 | 2.755862 | 2.084743 | 3.150786 | 3.195011 | 2.681374 |
| abcB10 | 0.751222 | 1.008967 | 0.949885 | 0.73771 | 1.629519 | 1.236184 | 1.235681 |
| abcB11 | 0.784444 | 2.16215 | 1.917978 | 1.702549 | 2.400461 | 1.975136 | 1.910758 |
| abcC1 | 0.895037 | 2.911508 | 2.292826 | 2.378907 | 3.16464 | 3.457143 | 3.371319 |
| abcC2 | 0.299319 | 0.167549 | 0.124114 | 0.115372 | 0.10115 | 0.095581 | 0.132 |
| abcC3 | 1.294998 | 2.436068 | 2.16669 | 2.213999 | 2.886399 | 2.877585 | 2.258469 |
| tubulin | 0.774576 | 0.785364 | 0.506652 | 0.97726 | 0.773432 | 1.16146 | 0.863422 |
| abcC4 | 1.587504 | 0.902162 | 0.76385 | 0.883164 | 1.672201 | 2.341406 | 1.209116 |
| abcC5 | 3.623535 | 3.510354 | 2.448125 | 3.3342 | 3.675962 | 4.986346 | 3.200455 |
| abcC6 | 2.007934 | 1.857434 | 1.570257 | 1.956011 | 2.558601 | 3.565862 | 2.262417 |
| abcC7 | 2.425941 | 2.557375 | 2.077849 | 2.41762 | 3.160601 | 5.103106 | 2.980384 |
| abcC8 | 0.151624 | 0.144998 | 0.079967 | 0.130703 | 0.150193 | 0.027386 | 0.056791 |
| abcC9 | 1.325183 | 1.894293 | 1.681861 | 1.999168 | 2.471505 | 2.936466 | 1.930869 |
| abcC10 | 1.269043 | 1.752618 | 1.221471 | 1.879453 | 2.025734 | 3.349735 | 1.955644 |
| abcC11 | 1.23627 | 2.110493 | 1.72745 | 2.255053 | 2.73184 | 3.89784 | 2.464787 |
| abcC12 | 1.03601 | 1.932404 | 1.415934 | 1.748193 | 2.339279 | 3.749658 | 1.840483 |
| abcC13 | 0.620419 | 1.5941 | 0.910482 | 1.220902 | 1.607429 | 2.927824 | 1.42116 |
| abcD1 | 1.215288 | 1.664805 | 1.24051 | 1.562307 | 2.005313 | 2.772448 | 1.798033 |
| abcD2 | 0.849769 | 1.181542 | 0.816577 | 0.931717 | 1.731701 | 1.516814 | 1.390542 |
| abcD3 | 1.145877 | 0.800949 | 0.695273 | 0.531527 | 1.372148 | 1.476788 | 0.923485 |
| gdh | 0.771407 | 0.396189 | 0.243024 | 0.395577 | 0.366373 | 0.803564 | 0.351658 |
| abcD4 | 2.132327 | 1.802682 | 1.571895 | 1.524425 | 2.310022 | 2.56848 | 1.486204 |
| abcE1 | 1.506131 | 1.401787 | 1.156524 | 1.049789 | 1.916738 | 2.572449 | 1.380368 |
| abcF1 | 1.390543 | 1.265726 | 1.10583 | 0.95469 | 1.882541 | 2.163066 | 1.641743 |
| abcF2 | 1.959809 | 1.566269 | 1.536065 | 1.61467 | 2.366987 | 3.098901 | 2.135612 |
| abcF3 | 1.630267 | 1.105128 | 1.352814 | 1.418096 | 2.144692 | 2.449596 | 1.677338 |
| abcG1 | 1.748694 | 0.747608 | 1.632436 | 1.638912 | 2.466449 | 2.801668 | 2.223585 |
| abcG2 | 1.293175 | 0.642073 | 1.317143 | 1.155471 | 2.104373 | 1.942463 | 1.38268 |
| abcG4 | 1.143673 | 0.791185 | 2.035725 | 2.337872 | 2.838111 | 4.759776 | 2.91807 |
| abcG5 | 0.411601 | 0.276478 | 0.578796 | 0.58565 | 1.061195 | 1.275059 | 1.253378 |
| abcG8 | 0.86748 | 0.352435 | 1.500072 | 1.439434 | 2.288885 | 3.098981 | 1.785673 |

TABLE 4

| | bt20 | caco2 | caov | colo320 | hbt161 | hek | hek2 | hepG2 |
|---|---|---|---|---|---|---|---|---|
| wt1 | 0.449935 | 0.385505 | 0.890929 | 0.304608 | 0.24495 | 0.446078 | 0.266343 | 0.387377 |
| abcA1 | 0.768097 | 0.797436 | 0.518441 | 0.621845 | 0.835439 | 0.810359 | 0.467852 | 0.582675 |
| abcA2 | 1.182087 | 1.253334 | 1.820371 | 1.093703 | 1.244025 | 1.385231 | 0.966688 | 1.047716 |
| abcA3 | 1.944963 | 2.036125 | 2.700699 | 1.915702 | 1.699915 | 1.925614 | 1.310134 | 1.651127 |
| abcA4 | 1.513128 | 1.767484 | 2.940329 | 1.687363 | 1.194481 | 2.012839 | 1.484887 | 1.623263 |
| abcA5 | 0.756045 | 0.821536 | 0.560633 | 0.708142 | 0.757119 | 0.820963 | 0.887827 | 0.772314 |
| abcA6 | 1.308388 | 1.427791 | 1.387689 | 1.701897 | 1.186286 | 1.757263 | 1.40119 | 1.266125 |
| abcA7 | 0.207828 | 0.345869 | 0.265016 | 0.198515 | 0.146791 | 0.317639 | 0.219484 | 0.400446 |
| abcA8 | 0.962841 | 1.050819 | 0.86643 | 1.218723 | 0.911364 | 1.121936 | 0.734216 | 1.103492 |
| abcA9 | 1.724345 | 1.871425 | 1.860309 | 2.769831 | 1.806107 | 2.371339 | 2.263215 | 2.426137 |
| abcA10 | 0.953953 | 1.17283 | 0.609213 | 1.324216 | 1.180485 | 1.049321 | 1.244112 | 1.047148 |
| abcA12 | 1.17641 | 1.440625 | 1.513381 | 1.60068 | 1.090711 | 1.466843 | 1.212869 | 1.117365 |
| abcB1 | 1.592455 | 1.649195 | 1.997379 | 1.803716 | 1.334924 | 1.746095 | 1.459475 | 1.075962 |
| abcB2 | 1.199418 | 1.351829 | 1.84043 | 1.266646 | 1.122756 | 1.550531 | 1.156466 | 1.157379 |
| actin | 0.719235 | 0.608562 | 1.33296 | 0.76097 | 0.391196 | 0.71818 | 0.66464 | 0.688709 |
| abcB3 | 1.486484 | 1.522236 | 2.630753 | 1.640275 | 1.299969 | 1.800863 | 1.736528 | 1.270251 |
| abcB4 | 1.964086 | 1.998066 | 4.415974 | 2.798798 | 1.625417 | 2.716562 | 2.568845 | 2.33386 |
| abcB6 | 1.044218 | 1.187463 | 1.780541 | 1.132177 | 0.886858 | 1.291633 | 1.244317 | 1.076702 |
| abcB7 | 0.018948 | 0.037458 | 0.011884 | 0.057523 | 0.018535 | 0.04761 | 0.050347 | 0.079079 |
| abcB8 | 1.703606 | 1.837848 | 6.054952 | 1.948856 | 1.826467 | 1.994679 | 2.05263 | 2.575888 |
| abcB9 | 1.749211 | 1.857188 | 3.042333 | 2.113523 | 1.354262 | 2.19237 | 2.152301 | 1.481011 |
| abcB10 | 0.722153 | 0.740217 | 0.711001 | 0.603722 | 0.754164 | 0.718677 | 0.68991 | 0.849325 |
| abcB11 | 1.260054 | 1.411866 | 1.583004 | 1.578301 | 1.29265 | 1.629405 | 1.335615 | 1.260157 |
| abcC1 | 1.625626 | 1.936328 | 2.063644 | 1.95517 | 1.709894 | 1.996602 | 1.881361 | 1.780451 |
| abcC2 | 0.088677 | 0.137505 | 0.169088 | 0.100733 | 0.07114 | 0.146131 | 0.096372 | 0.100066 |
| abcC3 | 1.537236 | 1.772482 | 3.719264 | 1.628826 | 1.322345 | 2.080873 | 1.436527 | 1.855849 |
| tubulin | 0.587555 | 0.49726 | 1.108869 | 0.61622 | 0.339106 | 0.659861 | 0.503339 | 0.475254 |
| abcC4 | 0.823182 | 0.802581 | 1.165193 | 0.680229 | 0.71466 | 0.837431 | 0.640674 | 0.784907 |
| abcC5 | 1.86398 | 2.024445 | 4.567212 | 2.228483 | 1.414696 | 2.308225 | 1.964943 | 1.82382 |
| abcC6 | 1.356916 | 1.489253 | 3.66764 | 1.546164 | 0.95395 | 1.771611 | 1.335531 | 1.453101 |
| abcC7 | 1.591259 | 1.433233 | 4.076304 | 1.950342 | 1.210689 | 1.880969 | 1.999273 | 1.353818 |
| abcC8 | 0.053494 | 0.074546 | 0.065545 | 0.104735 | 0.05018 | 0.11065 | 0.082263 | 0.033719 |
| abcC9 | 1.187419 | 1.242552 | 2.254089 | 1.195695 | 0.990398 | 1.443717 | 1.220749 | 1.289507 |
| abcC10 | 1.130296 | 1.178478 | 2.578573 | 1.066977 | 0.925706 | 1.216042 | 1.146042 | 1.284626 |
| abcC11 | 1.374725 | 1.432901 | 3.022153 | 1.711936 | 1.189867 | 1.73272 | 1.670084 | 1.410793 |
| abcC12 | 1.111969 | 1.214391 | 1.303228 | 1.386991 | 1.069708 | 1.335106 | 1.163744 | 1.110809 |
| abcC13 | 0.840939 | 0.905348 | 0.737543 | 0.950992 | 1.006877 | 1.089272 | 0.920626 | 1.004046 |
| abcD1 | 0.84992 | 0.926309 | 0.955786 | 1.056844 | 0.777017 | 1.15295 | 1.158508 | 0.92448 |
| abcD2 | 0.688086 | 0.703713 | 0.609123 | 0.591753 | 0.844588 | 0.688646 | 0.789255 | 0.65217 |
| abcD3 | 0.547074 | 0.525532 | 0.450643 | 0.504081 | 0.489374 | 0.580162 | 0.490847 | 0.528706 |
| gdh | 0.255624 | 0.238247 | 0.625588 | 0.262307 | 0.158692 | 0.224498 | 0.209865 | 0.372412 |
| abcD4 | 1.060798 | 0.925775 | 1.028076 | 1.258453 | 0.93342 | 1.184339 | 1.079328 | 0.76575 |
| abcE1 | 0.986998 | 0.960709 | 0.703003 | 1.025294 | 0.861156 | 0.993991 | 0.867632 | 0.805506 |
| abcF1 | 0.768642 | 0.756375 | 0.897542 | 0.678137 | 0.660569 | 0.771958 | 0.784113 | 0.74829 |
| abcF2 | 1.224471 | 1.296456 | 2.403678 | 1.226085 | 0.932391 | 1.399647 | 1.101049 | 1.214244 |
| abcF3 | 1.074713 | 0.960609 | 2.389467 | 1.089731 | 0.987102 | 1.05178 | 0.91834 | 1.157463 |
| abcG1 | 1.196602 | 1.202485 | 2.596537 | 1.300165 | 1.03185 | 1.374418 | 1.258006 | 1.302754 |
| abcG2 | 0.933321 | 0.858128 | 0.87909 | 0.848582 | 0.999213 | 0.99189 | 0.743274 | 0.871823 |
| abcG4 | 1.480268 | 1.722414 | 2.293729 | 2.036339 | 1.441704 | 1.864626 | 1.732034 | 1.268103 |
| abcG5 | 0.472781 | 0.706851 | 0.280027 | 0.486234 | 0.694468 | 0.618364 | 0.596478 | 0.661543 |
| abcG8 | 0.875052 | 1.052484 | 1.172665 | 1.053437 | 0.809421 | 1.121522 | 0.933028 | 0.908288 |
| | ht75 | ht177 | lncap | mcf7 | mda453 | mda468 | mfe29c | skmes1 |
| wt1 | 0.308181 | 0.304056 | 0.21599 | 0.412246 | 0.508213 | 0.295845 | 0.342594 | 0.550415 |
| abcA1 | 0.762017 | 0.834926 | 0.731709 | 0.782136 | 0.738906 | 0.709946 | 0.845089 | 0.753074 |
| abcA2 | 1.039953 | 1.265724 | 0.954405 | 1.274588 | 1.509415 | 1.018316 | 1.351741 | 1.201496 |
| abcA3 | 1.615127 | 2.455845 | 1.658265 | 1.975279 | 2.23138 | 1.47321 | 2.149978 | 1.84459 |
| abcA4 | 1.236765 | 1.549109 | 1.163562 | 1.654144 | 2.092621 | 1.227284 | 2.049501 | 1.826623 |
| abcA5 | 1.10859 | 0.703534 | 0.782537 | 0.771315 | 0.744159 | 0.940792 | 0.786357 | 0.889053 |
| abcA6 | 1.235541 | 1.400189 | 1.229131 | 1.411772 | 1.909291 | 1.156829 | 1.554603 | 1.527496 |
| abcA7 | 0.866515 | 0.244047 | 0.155622 | 0.194454 | 0.180275 | 0.357642 | 0.259175 | 0.301174 |
| abcA8 | 1.036426 | 0.998093 | 0.924495 | 1.084405 | 1.14006 | 0.954594 | 0.97242 | 1.037492 |
| abcA9 | 1.623488 | 2.350293 | 1.86799 | 1.936104 | 2.723096 | 1.536668 | 2.378677 | 1.565904 |
| abcA10 | 0.91574 | 1.527891 | 1.18511 | 0.889702 | 1.22027 | 0.948222 | 1.347226 | 0.628905 |
| abcA12 | 1.019292 | 1.472441 | 1.065568 | 1.294512 | 1.48225 | 1.008609 | 1.566924 | 1.117011 |
| abcB1 | 1.193674 | 1.732081 | 1.353098 | 1.594457 | 2.026945 | 1.242052 | 1.687249 | 1.541535 |
| abcB2 | 1.11972 | 1.416726 | 0.926375 | 1.294342 | 1.427115 | 1.096295 | 1.167458 | 1.229339 |
| actin | 0.561311 | 0.777712 | 0.293154 | 0.6631 | 1.001333 | 0.445075 | 0.694403 | 0.651497 |
| abcB3 | 1.31732 | 1.539448 | 1.125258 | 1.595088 | 1.797814 | 1.342134 | 1.335174 | 1.707363 |
| abcB4 | 1.743798 | 2.404511 | 1.580001 | 2.07431 | 2.734892 | 1.686318 | 2.170364 | 1.880807 |
| abcB6 | 1.097663 | 1.179461 | 0.839762 | 1.093713 | 1.203301 | 0.989815 | 1.117045 | 1.093286 |
| abcB7 | 0.192395 | 0.025819 | 0.027473 | 0.02745 | 0.047503 | 0.060968 | 0.053285 | 0.026371 |
| abcB8 | 1.70929 | 2.044158 | 1.584723 | 2.028373 | 2.287358 | 1.595324 | 1.705755 | 1.774655 |
| abcB9 | 1.381805 | 2.144353 | 1.353503 | 1.883984 | 2.285295 | 1.450828 | 2.111165 | 1.652774 |
| abcB10 | 0.864966 | 0.673941 | 0.70583 | 0.731902 | 0.709068 | 0.898124 | 0.648771 | 0.856905 |
| abcB11 | 1.112044 | 1.929102 | 1.204959 | 1.232124 | 1.713976 | 1.127441 | 1.773707 | 1.096688 |
| abcC1 | 1.440811 | 2.583729 | 1.715036 | 1.694672 | 2.138049 | 1.476341 | 2.335579 | 1.271714 |
| abcC2 | 0.34897 | 0.108538 | 0.072665 | 0.087209 | 0.111046 | 0.083661 | 0.088944 | 0.097987 |
| abcC3 | 1.276355 | 1.743071 | 1.171506 | 1.681642 | 2.213411 | 1.28337 | 1.843649 | 1.371781 |
| tubulin | 0.368376 | 0.691328 | 0.294136 | 0.512403 | 0.774917 | 0.359841 | 0.563246 | 0.517905 |
| abcC4 | 0.817642 | 0.816608 | 0.742674 | 0.834712 | 0.885097 | 0.898167 | 0.714499 | 0.847414 |
| abcC5 | 1.761976 | 2.449876 | 1.406791 | 1.853741 | 2.443766 | 1.651213 | 2.278364 | 1.862821 |
| abcC6 | 1.180481 | 1.663072 | 1.011199 | 1.39548 | 1.755264 | 1.191755 | 1.486006 | 1.304483 |
| abcC7 | 1.397619 | 1.797035 | 1.25563 | 1.784319 | 2.066094 | 1.412336 | 1.708986 | 1.664851 |
| abcC8 | 0.21222 | 0.065425 | 0.073289 | 0.063254 | 0.071958 | 0.106965 | 0.094554 | 0.067353 |
| abcC9 | 1.048617 | 1.34412 | 0.985137 | 1.285571 | 1.411054 | 1.112097 | 1.255279 | 1.218112 |
| abcC10 | 1.094796 | 1.139316 | 0.889627 | 1.038115 | 1.249872 | 1.031099 | 1.180342 | 1.153737 |
| abcC11 | 1.022198 | 1.706622 | 1.160932 | 1.510989 | 1.912769 | 1.171623 | 1.715948 | 1.358642 |
| abcC12 | 0.987967 | 1.681499 | 1.113501 | 1.052039 | 1.489858 | 1.100018 | 1.616219 | 0.903452 |
| abcC13 | 0.83562 | 1.133407 | 1.025386 | 0.736461 | 1.047915 | 0.861287 | 0.839939 | 0.436087 |
| abcD1 | 0.895484 | 1.136283 | 0.72933 | 0.927099 | 1.111196 | 0.935983 | 1.081435 | 0.792278 |
| abcD2 | 0.863113 | 0.752442 | 0.758516 | 0.713474 | 0.728358 | 0.871361 | 0.6374 | 0.832836 |
| abcD3 | 0.719779 | 0.417957 | 0.497679 | 0.528641 | 0.488268 | 0.709882 | 0.420669 | 0.612702 |
| gdh | 0.261956 | 0.189704 | 0.14287 | 0.24899 | 0.433437 | 0.216801 | 0.272488 | 0.253208 |
| abcD4 | 1.221608 | 0.56994 | 1.000022 | 1.064614 | 1.238436 | 1.201605 | 1.251406 | 1.027184 |
| abcE1 | 0.989601 | 0.65678 | 0.910156 | 0.94905 | 0.991174 | 0.995129 | 0.951503 | 0.790204 |
| abcF1 | 1.042161 | 0.595922 | 0.653341 | 0.728378 | 0.718025 | 1.006292 | 0.653326 | 0.822604 |
| abcF2 | 1.146714 | 0.727873 | 0.940822 | 1.236699 | 1.447004 | 1.152113 | 1.277433 | 1.183414 |
| abcF3 | 1.062895 | 0.774609 | 0.970993 | 1.101751 | 1.148605 | 1.137756 | 1.1244 | 1.056777 |
| abcG1 | 1.073128 | 0.926339 | 0.942114 | 1.224536 | 1.348379 | 1.120749 | 1.279799 | 1.168968 |
| abcG2 | 1.080044 | 0.697082 | 0.986597 | 1.080013 | 0.854118 | 1.175995 | 0.921388 | 1.1588 |
| abcG4 | 1.198349 | 1.410631 | 1.405131 | 1.924701 | 2.05888 | 1.37345 | 1.961488 | 1.259826 |
| abcG5 | 1.239435 | 0.624966 | 0.610113 | 0.529322 | 0.419736 | 0.608818 | 0.50803 | 0.29096 |
| abcG8 | 1.136554 | 0.902918 | 0.87186 | 0.97718 | 0.736492 | 1.116261 | 1.010079 | 0.918548 |

| | sknas | sknbe | sknd2 | sknmc | t47d | zr75 | mdck |
|---|---|---|---|---|---|---|---|
| wt1 | 0.578356 | 0.389629 | 0.325984 | 0.454171 | 0.312887 | 0.687244 | 0.284422 |
| abcA1 | 1.433692 | 0.737498 | 0.679857 | 0.6579 | 0.774974 | 1.08654 | 0.494096 |
| abcA2 | 1.276373 | 1.14536 | 1.156883 | 1.324679 | 1.183162 | 1.597367 | 0.804096 |
| abcA3 | 2.764568 | 2.067384 | 1.87063 | 1.895904 | 1.652789 | 2.277643 | 1.033394 |
| abcA4 | 1.143706 | 1.65426 | 1.573126 | 1.990101 | 1.381863 | 2.284975 | 1.056805 |
| abcA5 | 1.135408 | 0.768629 | 0.756673 | 0.679619 | 0.894302 | 0.986069 | 0.641177 |
| abcA6 | 1.369658 | 1.481916 | 1.39639 | 1.494469 | 1.313904 | 1.582144 | 0.927666 |
| abcA7 | 1.080252 | 0.203865 | 0.188959 | 0.215634 | 0.179256 | 0.267168 | 0.207623 |
| abcA8 | 1.349928 | 1.025242 | 0.886714 | 0.847255 | 1.022246 | 1.235427 | 0.670468 |
| abcA9 | 1.354766 | 2.526675 | 2.327525 | 2.156105 | 1.708472 | 1.940309 | 1.486049 |
| abcA10 | 0.688971 | 1.270371 | 0.973263 | 1.04751 | 1.046113 | 1.335579 | 1.065581 |
| abcA12 | 0.990535 | 1.331292 | 1.190444 | 1.392408 | 1.118277 | 1.739784 | 1.192578 |
| abcB1 | 1.564133 | 1.677968 | 1.615571 | 1.758492 | 1.401194 | 2.141621 | 1.267873 |
| abcB2 | 2.337697 | 1.092601 | 1.568935 | 1.102826 | 1.174975 | 2.377612 | 1.014469 |
| actin | 1.197666 | 0.758387 | 0.8249 | 0.793138 | 0.461449 | 0.52997 | 0.572577 |
| abcB3 | 1.97714 | 1.632669 | 1.871517 | 1.236215 | 1.412211 | 1.730083 | 1.459115 |
| abcB4 | 2.88788 | 2.617826 | 2.903345 | 2.237082 | 1.77387 | 2.148607 | 1.451544 |
| abcB6 | 1.45607 | 1.140998 | 1.370637 | 1.041213 | 1.046085 | 1.028666 | 1.059108 |
| abcB7 | 0.03789 | 0.052285 | 0.059435 | 0.07371 | 0.035314 | 0.023789 | 0.04114 |
| abcB8 | 1.718819 | 2.137902 | 2.289526 | 1.609317 | 1.65878 | 1.581217 | 1.844647 |
| abcB9 | 2.617776 | 1.81264 | 2.273309 | 1.653489 | 1.453927 | 1.693261 | 1.535293 |
| abcB10 | 0.899713 | 0.765187 | 0.78356 | 0.585106 | 0.75194 | 0.655141 | 0.707522 |
| abcB11 | 0.939503 | 1.639746 | 1.582139 | 1.350356 | 1.107691 | 1.046764 | 1.094056 |
| abcC1 | 1.071956 | 2.20805 | 1.891352 | 1.886802 | 1.46032 | 1.832184 | 1.930339 |
| abcC2 | 0.358484 | 0.127067 | 0.102382 | 0.091506 | 0.046676 | 0.050655 | 0.07558 |
| abcC3 | 1.550976 | 1.847483 | 1.787302 | 1.756007 | 1.331926 | 1.525035 | 1.293147 |
| tubulin | 0.927684 | 0.59561 | 0.417937 | 0.775102 | 0.356899 | 0.61554 | 0.494376 |
| abcC4 | 1.9013 | 0.684188 | 0.6301 | 0.700471 | 0.771636 | 1.240876 | 0.692312 |
| abcC5 | 4.339787 | 2.662207 | 2.019458 | 2.644482 | 1.696269 | 2.642616 | 1.832506 |
| abcC6 | 2.404835 | 1.408654 | 1.295305 | 1.551387 | 1.180664 | 1.889802 | 1.295408 |
| abcC7 | 2.905468 | 1.93948 | 1.714017 | 1.917507 | 1.458457 | 2.704496 | 1.706499 |
| abcC8 | 0.181595 | 0.109965 | 0.065965 | 0.103665 | 0.069306 | 0.014514 | 0.032517 |
| abcC9 | 1.587128 | 1.436607 | 1.387367 | 1.585616 | 1.140474 | 1.55624 | 1.105571 |
| abcC10 | 1.51989 | 1.329163 | 1.007591 | 1.490666 | 0.934773 | 1.775261 | 1.119757 |
| abcC11 | 1.480639 | 1.600571 | 1.424973 | 1.788569 | 1.260605 | 2.06574 | 1.41128 |
| abcC12 | 1.240795 | 1.46551 | 1.168004 | 1.386559 | 1.079458 | 1.987208 | 1.053818 |
| abcC13 | 0.743055 | 1.208944 | 0.751056 | 0.968344 | 0.741747 | 1.55166 | 0.813723 |
| abcD1 | 1.455509 | 1.262566 | 1.023296 | 1.239125 | 0.92535 | 1.469316 | 1.029512 |
| abcD2 | 1.01774 | 0.896066 | 0.673594 | 0.73898 | 0.799092 | 0.803867 | 0.796192 |
| abcD3 | 1.372378 | 0.60743 | 0.573531 | 0.421575 | 0.633177 | 0.782654 | 0.528766 |
| gdh | 0.923888 | 0.300465 | 0.20047 | 0.313747 | 0.169063 | 0.425865 | 0.201351 |
| abcD4 | 2.553816 | 1.367131 | 1.296656 | 1.20908 | 1.065958 | 1.361219 | 0.850966 |
| abcE1 | 1.803843 | 1.063097 | 0.954016 | 0.832628 | 0.884477 | 1.363322 | 0.790367 |
| abcF1 | 1.665407 | 0.959911 | 0.912198 | 0.757201 | 0.868697 | 1.146361 | 0.940024 |
| abcF2 | 2.347198 | 1.187838 | 1.2671 | 1.280657 | 1.092244 | 1.642326 | 1.222802 |
| abcF3 | 1.952517 | 0.838114 | 1.115936 | 1.124746 | 0.989666 | 1.298214 | 0.960405 |
| abcG1 | 2.094352 | 0.566976 | 1.346596 | 1.299883 | 1.138141 | 1.484801 | 1.273173 |
| abcG2 | 1.548793 | 0.48694 | 1.086511 | 0.916448 | 0.971061 | 1.029448 | 0.791691 |
| abcG4 | 1.369739 | 0.600024 | 1.679269 | 1.854256 | 1.309644 | 2.522541 | 1.670819 |
| abcG5 | 0.49296 | 0.209677 | 0.477449 | 0.464501 | 0.489687 | 0.675744 | 0.717655 |
| abcG8 | 1.038951 | 0.267282 | 1.237409 | 1.14167 | 1.056204 | 1.642369 | 1.022435 |

TABLE 5

| | dox 0 h | dox 2 h | dox 4 h | dox 8 h |
|---|---|---|---|---|
| abcA1 | 1.793411 | 3.052731 | 1.865644 | 2.34586 |
| abcA2 | 3.394744 | 6.223801 | 2.94659 | 4.02209 |
| abcA3 | 4.445693 | 8.071446 | 4.290698 | 5.179128 |
| abcA4 | 5.098287 | 8.764862 | 4.534571 | 6.09907 |
| abcA5 | 2.006987 | 3.30202 | 2.020768 | 2.451236 |
| abcA6 | 3.567858 | 6.044507 | 3.366697 | 4.295772 |
| abcA7 | 0.906336 | 1.841564 | 0.932998 | 0.935086 |
| abcA8 | 1.575163 | 3.035544 | 1.785517 | 2.17441 |
| abcA9 | 5.12988 | 7.825115 | 4.816535 | 5.72013 |
| abcA10 | 3.225933 | 4.820089 | 3.418986 | 3.792907 |
| abcA12 | 3.485887 | 5.828746 | 3.418674 | 4.195394 |
| abcB1 | 3.658465 | 6.734501 | 3.865342 | 4.758501 |
| abcB2 | 2.792672 | 5.067235 | 3.714749 | 4.008349 |
| abcB3 | 3.312315 | 6.838271 | 4.325461 | 4.812997 |
| abcB4 | 5.149497 | 9.148426 | 5.624165 | 6.417042 |
| abcB6 | 2.795918 | 5.173665 | 3.283246 | 3.61177 |
| abcB7 | 0.143706 | 0.262199 | 0.161948 | 0.176608 |
| abcB8 | 4.688411 | 8.003626 | 5.437681 | 5.35345 |
| abcB9 | 4.532227 | 8.387881 | 4.897126 | 5.522502 |
| abcB10 | 1.264095 | 2.115507 | 1.484423 | 1.542031 |
| abcB11 | 3.285622 | 5.310097 | 3.404054 | 3.888131 |
| abcC1 | 4.397451 | 7.004924 | 4.767338 | 5.055766 |
| abcC2 | 0.340701 | 0.614144 | 0.360272 | 0.354806 |
| abcC3 | 4.024623 | 7.155717 | 4.013536 | 4.199702 |
| abcC4 | 1.480616 | 2.612061 | 2.089878 | 2.285607 |
| abcC5 | 5.251928 | 10.50642 | 6.290367 | 6.521707 |
| abcC6 | 3.94515 | 7.696336 | 4.515506 | 4.899487 |
| abcC7 | 3.904822 | 7.480766 | 4.5794 | 5.093014 |
| abcC8 | 0.210057 | 0.322881 | 0.243749 | 0.22546 |
| abcC9 | 3.239867 | 5.598434 | 3.67832 | 3.981505 |
| abcC10 | 3.504958 | 5.15091 | 3.334564 | 3.632591 |
| abcC11 | 4.300962 | 7.608052 | 4.383947 | 5.056108 |
| abcC12 | 2.421183 | 5.226012 | 3.53205 | 3.976487 |
| abcC13 | 2.231485 | 3.20307 | 2.54815 | 2.593022 |
| abcD1 | 2.923938 | 4.476831 | 3.385873 | 3.516307 |
| abcD2 | 1.810003 | 2.503156 | 2.516228 | 2.409319 |
| abcD3 | 1.143253 | 2.09855 | 1.78719 | 1.733079 |
| abcD4 | 2.411452 | 4.360857 | 3.102722 | 3.194107 |
| abcE1 | 2.060757 | 4.155317 | 2.79372 | 3.087661 |
| abcF1 | 1.969904 | 2.485367 | 2.869902 | 2.661525 |
| abcF2 | 3.671255 | 5.978677 | 4.068172 | 4.806913 |
| abcF3 | 2.398669 | 3.920654 | 2.794743 | 2.793001 |
| abcG1 | 3.224847 | 5.471919 | 3.555021 | 3.838933 |
| abcG2 | 1.711538 | 2.988958 | 2.136826 | 2.080252 |
| abcG4 | 5.107502 | 9.589581 | 5.308586 | 6.270866 |
| abcG5 | 1.427298 | 2.200836 | 1.76435 | 1.751627 |
| abcG8 | 2.379986 | 4.696989 | 2.811059 | 2.9413 |
| standard | 11 | 11 | 11 | 11 |

TABLE 6

| | vin0 h | vin2 h | vin4 h | vin 8 h |
|---|---|---|---|---|
| abcA1 | 6.033981 | 6.833133 | 5.063992 | 6.364167 |
| abcA2 | 6.092914 | 7.398232 | 6.087334 | 9.54274 |
| abcA3 | 8.389483 | 10.83098 | 9.241369 | 14.93551 |
| abcA4 | 8.853516 | 11.00906 | 9.361 | 15.90913 |
| abcA5 | 7.368576 | 7.894724 | 6.136832 | 8.641923 |
| abcA6 | 8.249337 | 9.900094 | 7.850423 | 14.54007 |
| abcA7 | 3.030993 | 2.265104 | 1.807985 | 3.13492 |
| abcA8 | 6.552532 | 8.424365 | 6.723552 | 10.25917 |
| abcA9 | 10.06712 | 12.25496 | 10.20161 | 18.62193 |
| abcA10 | 7.746441 | 10.27696 | 8.658689 | 10.49502 |
| abcA12 | 6.787256 | 8.473897 | 6.774483 | 8.088676 |
| abcB1 | 9.188582 | 12.11622 | 9.658148 | 11.52253 |
| abcB2 | 6.87262 | 8.667108 | 8.938251 | 8.490746 |
| abcB3 | 8.053858 | 9.144091 | 8.399529 | 14.78834 |
| abcB4 | 11.54316 | 16.19463 | 13.24367 | 23.08427 |
| abcB6 | 6.745944 | 8.654504 | 8.468344 | 7.559171 |
| abcB7 | 0.969163 | 0.461908 | 0.346696 | 0.695811 |
| abcB8 | 12.78564 | 16.52333 | 15.3408 | 20.22378 |
| abcB9 | 9.119206 | 12.61687 | 12.27524 | 12.23283 |
| abcB10 | 4.908251 | 4.258718 | 3.989921 | 5.497747 |
| abcB11 | 5.160003 | 7.105947 | 7.457957 | 9.052987 |
| abcC1 | 11.60798 | 16.22045 | 15.1016 | 12.68866 |
| abcC2 | 2.150775 | 1.429625 | 1.196853 | 1.419261 |
| abcC3 | 9.195576 | 12.14918 | 10.16914 | 11.28798 |
| abcC4 | 5.334611 | 5.277331 | 5.742927 | 6.273209 |
| abcC5 | 9.43692 | 10.94563 | 12.64342 | 17.32233 |
| abcC6 | 7.241802 | 8.103238 | 8.42442 | 9.917254 |
| abcC7 | 11.61626 | 13.77896 | 13.42656 | 16.12312 |
| abcC8 | 0.527266 | 0.353293 | 0.453532 | 0.880544 |
| abcC9 | 7.904892 | 9.880234 | 9.013857 | 9.735132 |
| abcC10 | 8.724349 | 10.91895 | 8.808216 | 9.006965 |
| abcC11 | 8.108411 | 9.016067 | 7.820571 | 11.10364 |
| abcC12 | 7.448083 | 8.04745 | 7.567471 | 8.523092 |
| abcC13 | 7.931162 | 9.550546 | 8.963819 | 7.491041 |
| abcD1 | 8.371099 | 10.01996 | 9.667713 | 8.253821 |
| abcD2 | 6.641271 | 6.669278 | 6.511982 | 5.888302 |
| abcD3 | 5.873059 | 5.918177 | 5.802474 | 5.647165 |
| abcD4 | 7.456156 | 8.051998 | 8.68655 | 10.27714 |
| abcE1 | 6.417 | 6.427651 | 6.814744 | 7.53135 |
| abcF1 | 7.793391 | 7.85194 | 7.234306 | 5.758539 |
| abcF2 | 7.663223 | 9.20984 | 8.64578 | 9.39799 |
| abcF3 | 8.043509 | 8.975626 | 7.819995 | 8.132801 |
| abcG1 | 7.649376 | 9.753252 | 8.36374 | 8.726646 |
| abcG2 | 5.486492 | 6.441959 | 5.659027 | 6.036638 |
| abcG4 | 9.481624 | 12.50021 | 13.2242 | 9.999301 |
| abcG5 | 5.764325 | 6.024204 | 6.084062 | 4.098926 |
| abcG8 | 7.991649 | 9.698668 | 8.750869 | 7.315558 |
| standard | 24 | 24 | 24 | 24 |

1-46. (canceled)

47. An array comprising two or more nucleic acid molecules immobilized on a substrate, wherein the two or more nucleic acid molecules each comprise a sequence that hybridizes to one ATP-binding cassette (ABC) transporter gene.

48. The array according to claim 47, wherein the two or more nucleic acid molecules each comprise a portion of the 3′ untranslated region of the ABC transporter gene.

49. The array according to claim 47, wherein the two or more nucleic acid molecules each comprise a nucleic acid sequence selected from:
(a) the nucleic acid sequences as shown in SEQ ID NOS: 1 to 47 and FIGS. 1 to 47, wherein T can also be U;
(b) nucleic acid sequences complementary to (a);
(c) nucleic acid sequences which are homologous to (a) or (b); or
(d) a fragment of (a) to (c), which comprises a sequence that hybridizes to one of the ABC transporter genes.

50. The array according to claim 47, wherein the array is a microarray.

51. A method of detecting the expression of two or more ATP-binding cassette (ABC) transporter genes, comprising the steps:
(a) providing two or more nucleic acid molecules, each comprising a sequence that hybridizes to one ABC transporter gene;
(b) providing transcription indicators from a test sample;
(c) allowing the transcription indicators to hybridize with said two or more nucleic acid molecules; and
(d) detecting hybridization of said transcription indicators with said two or more nucleic acid molecules.
wherein hybridization is indicative of the expression of the ABC transporter genes.

52. The method according to claim 51, wherein the two or more nucleic acid molecules each comprise a portion of the 3′ untranslated region of the ABC transporter gene.

53. The method according to claim 51, wherein the two or more nucleic acid molecules each comprise a nucleic acid sequence selected from:
(a) the nucleic acid sequences as shown in SEQ ID NOS: 1 to 47 and FIGS. 1 to 47, wherein T can also be U;
(b) nucleic acid sequences complementary to (a);
(c) nucleic acid sequences which are homologous to (a) or (b); or
(d) a fragment of (a) to (c), which comprises a sequence that hybridizes to one of the ABC transporter genes.

54. The method according to claim 51, wherein the two or more nucleic acid molecules that each comprise a sequence that hybridizes to one ABC transporter gene, are prepared using PCR and primer pairs, wherein the primer pairs comprise a nucleic acid sequence selected from one or more of the group comprising:
(a) one or more isolated and purified pairs of nucleic acid sequences selected from:
SEQ ID NO: 48 and SEQ ID NO: 49;
SEQ ID NO: 50 and SEQ ID NO: 51;
SEQ ID NO: 52 and SEQ ID NO: 53;
SEQ ID NO: 54 and SEQ ID NO: 55;
SEQ ID NO: 56 and SEQ ID NO: 57;
SEQ ID NO: 58 and SEQ ID NO: 59;
SEQ ID NO: 60 and SEQ ID NO: 61;
SEQ ID NO: 62 and SEQ ID NO: 63;
SEQ ID NO: 64 and SEQ ID NO: 65;
SEQ ID NO: 66 and SEQ ID NO: 67;
SEQ ID NO: 68 and SEQ ID NO: 69;
SEQ ID NO: 70 and SEQ ID NO: 71;
SEQ ID NO: 72 and SEQ ID NO: 73;
SEQ ID NO: 74 and SEQ ID NO: 75;
SEQ ID NO: 76 and SEQ ID NO: 77;
SEQ ID NO: 78 and SEQ ID NO: 79;
SEQ ID NO: 80 and SEQ ID NO: 81;
SEQ ID NO: 82 and SEQ ID NO: 83;
SEQ ID NO: 84 and SEQ ID NO: 85;
SEQ ID NO: 86 and SEQ ID NO: 87;
SEQ ID NO: 88 and SEQ ID NO: 89;
SEQ ID NO: 90 and SEQ ID NO: 91;
SEQ ID NO: 92 and SEQ ID NO: 93;
SEQ ID NO: 94 and SEQ ID NO: 95;
SEQ ID NO: 96 and SEQ ID NO: 97;
SEQ ID NO: 98 and SEQ ID NO: 99;
SEQ ID NO: 100 and SEQ ID NO: 101;
SEQ ID NO: 102 and SEQ ID NO: 103;
SEQ ID NO: 104 and SEQ ID NO: 105;
SEQ ID NO: 106 and SEQ ID NO: 107;
SEQ ID NO: 108 and SEQ ID NO: 109;
SEQ ID NO: 110 and SEQ ID NO: 111;
SEQ ID NO: 112 and SEQ ID NO: 113;
SEQ ID NO: 114 and SEQ ID NO: 115;
SEQ ID NO: 116 and SEQ ID NO: 117;
SEQ ID NO: 118 and SEQ ID NO: 119;
SEQ ID NO: 120 and SEQ ID NO: 121;
SEQ ID NO: 122 and SEQ ID NO: 123;
SEQ ID NO: 124 and SEQ ID NO: 125;
SEQ ID NO: 126 and SEQ ID NO: 127;
SEQ ID NO: 128 and SEQ ID NO: 129;
SEQ ID NO: 130 and SEQ ID NO: 131;
SEQ ID NO: 132 and SEQ ID NO: 133;
SEQ ID NO: 134 and SEQ ID NO: 135;
SEQ ID NO: 136 and SEQ ID NO: 137;
SEQ ID NO: 138 and SEQ ID NO: 139; and
SEQ ID NO: 140 and SEQ ID NO: 141;
(b) the nucleic acid sequences in (a) wherein T can also be U;
(c) nucleic acid sequences complementary to (a) or (b); and
(d) nucleic acid sequences which are homologous to (a), (b) or (c).

55. The method according to claim 51 wherein the transcription indicators are selected from the group consisting of transcripts of the gene or genes, cDNA reverse transcribed from the transcript, cRNA transcribed from the cDNA, DNA amplified from the genes, and RNA transcribed from amplified DNA.

56. The method according to claim 51 performed in microarray format.

57. The method according to claim 51, further comprising the steps of:
(a) generating a set of expression data;
(b) storing the data in a database; and
(c) performing comparative analysis on the set of expression data, thereby analyzing ABC transporter gene expression.

58. A method for screening compounds for their effect on the expression of one or more ATP-binding cassette (ABC) transporter genes comprising:
(a) exposing a test sample to one or more compounds;
(b) providing a transcription indicator from the test sample;
(c) providing one or more nucleic acid sequences, each comprising a sequence that hybridizes to one ABC transporter gene;
(d) allowing said transcription inhibitor to hybridize with said one or more nucleic acid sequences; and
(e) detecting hybridization of said transcription indicator with said one or more nucleic acid sequences,
wherein hybridization is indicative of expression of the one or more ABC transporter gene expression.

59. The method according to claim 58 further comprising the step of quantitatively or qualitatively comparing the hybridization detected in step (e) with the hybridization of transcription indicators from a control sample, thereby determining the effect of the one or more compounds on the expression of the one or more ABC transporter genes.

60. A method for screening compounds for their effect on the expression of one or more ATP-binding cassette (ABC) transporter genes comprising:
(a) preparing an ABC transporter gene expression profile, using a method according to claim 51, of a test sample that has been exposed to one or more compounds;
(b) preparing an ABC transporter gene expression profile, using a method according to claim 51, of a control sample; and
(c) quantitatively or qualitatively comparing the gene expression profiles from (a) and (b),
wherein differential expression profiles in (a) and (b) is indicative of a compound having an effect on the expression of one or more ABC transporter genes.

61. The method according to claim 60, wherein if the expression of one or more of the ABC transporter genes in the test sample is increased compared to the control sample, then the efficacy of the one or more compounds may be decreased.

62. The method according to claim 60, wherein if the expression of one or more of ABC B1 (MDR1), ABC C1 (MRP1), ABC C2 (MRP2), and ABC G2 (BCRP) in the test sample is increased compared to the control sample, then the efficacy of the one or more compounds may be decreased.

63. The method according to claim 60, wherein if the expression of one or more of the ABC transporter genes in the test sample is decreased compared to the control sample, then the efficacy or toxicity of the one or more compounds may be increased.

64. The method according to claim 60, wherein if the expression of one or more of ABC B1 (MDR1), ABC C1 (MRP1), ABC C2 (MRP2), and ABC G2 (BCRP) in the test sample is decreased compared to the control sample, then the efficacy and/or toxicity of the one or more compounds may be increased.

65. A method of assessing the toxicity and/or efficacy of a compound in a subject comprising:
(a) preparing an ATP-binding cassette (ABC) transporter gene expression profile, using a method according to claim 51, of a test sample that has been exposed to the compound;
(b) preparing an ABC transporter gene expression profile, using a method according to claim 51, of a control sample; and
(c) quantitatively or qualitatively comparing the gene expression profiles from (a) and (b),
wherein a difference in the ABC transporter gene expression profiles in (a) and (b) is indicative of the toxicity and/or efficacy of the compound.

66. A method for determining a change in ATP-binding cassette (ABC) transporter gene expression profile for a compound in the presence of one or more different compounds comprising:
(a) preparing an ABC transporter gene expression profile, using a method according to claim 51, of a test sample that has been exposed to the compound;
(b) preparing an ABC transporter gene expression profile, using a method according to claim 51, of a test sample that has been exposed to the compound and the one or more different compounds; and
(c) quantitatively or qualitatively comparing the gene expression profile in (a) and (b),
wherein differential expression in (a) and (b) indicates that the ABC transporter gene expression profile of the compound changes in the presence of the one or more different compounds.

67. The method according to claim 66, wherein changes in the ABC transporter gene expression profile indicate the presence of drug-drug interactions.

68. The method according to claim 58 wherein the hybridization is detected over a period of time at specified time intervals.

69. A kit, comprising one or more of the following: a nucleic acid array according to claim 47, reagents for use with the arrays, signal detection and array-processing instruments, gene expression databases or analysis and database management software.

70. A relational database comprising ATP-binding cassette (ABC) transporter gene expression profiles obtained using the method according to claim 51.

71. Two or more isolated nucleic acid molecules, wherein each of the nucleic acid molecules comprises a sequence that hybridizes to one ATP-binding cassette (ABC) transporter gene.

72. The two or more isolated nucleic acid molecules according to claim 71, wherein each of the nucleic acid molecules comprise a portion of the 3′ untranslated region of the ABC transporter gene.

73. The two or more isolated nucleic acid molecules according to claim 71, wherein each of the nucleic acid molecules comprise a nucleic acid sequence selected from:
(a) the nucleic acid sequences as shown in SEQ ID NOS: 1 to 47 and FIGS. 1 to 47, wherein T can also be U;
(b) nucleic acid sequences complementary to (a);
(c) nucleic acid sequences which are homologous to (a) or (b); or
(d) a fragment of (a) to (c), which comprises a sequence that hybridizes to one of the ABC transporter genes.

74. Two or more pairs of primers for preparing the two or more nucleic acid molecules according to claim 71.

75. Two or more pairs of primers according to claim 74, wherein the primers comprise a nucleic acid sequence selected from the group consisting of:
(a) a nucleic acid sequence as shown in SEQ ID NOS: 48 to 141 and Table 1, wherein T can also be U;
(b) nucleic acid sequences complementary to (a); and
(c) nucleic acid sequences which are homologous to (a) or (b).

76. Two or more pairs of primers, wherein the primer pairs comprise a nucleic acid sequence selected from one or more of:
(a) one or more isolated and purified pairs of nucleic acid sequences selected from:
SEQ ID NO: 48 and SEQ ID NO: 49;
SEQ ID NO: 50 and SEQ ID NO: 51;
SEQ ID NO: 52 and SEQ ID NO: 53;
SEQ ID NO: 54 and SEQ ID NO: 55;
SEQ ID NO: 56 and SEQ ID NO: 57;
SEQ ID NO: 58 and SEQ ID NO: 59;
SEQ ID NO: 60 and SEQ ID NO: 61;
SEQ ID NO: 62 and SEQ ID NO: 63;
SEQ ID NO: 64 and SEQ ID NO: 65;
SEQ ID NO: 66 and SEQ ID NO: 67;
SEQ ID NO: 68 and SEQ ID NO: 69;
SEQ ID NO: 70 and SEQ ID NO: 71;
SEQ ID NO: 72 and SEQ ID NO: 73;
SEQ ID NO: 74 and SEQ ID NO: 75;
SEQ ID NO: 76 and SEQ ID NO: 77;
SEQ ID NO: 78 and SEQ ID NO: 79;
SEQ ID NO: 80 and SEQ ID NO: 81;
SEQ ID NO: 82 and SEQ ID NO: 83;
SEQ ID NO: 84 and SEQ ID NO: 85;
SEQ ID NO: 86 and SEQ ID NO: 87;
SEQ ID NO: 88 and SEQ ID NO: 89;
SEQ ID NO: 90 and SEQ ID NO: 91;
SEQ ID NO: 92 and SEQ ID NO: 93;
SEQ ID NO: 94 and SEQ ID NO: 95;
SEQ ID NO: 96 and SEQ ID NO: 97;
SEQ ID NO: 98 and SEQ ID NO: 99;
SEQ ID NO: 100 and SEQ ID NO: 101;
SEQ ID NO: 102 and SEQ ID NO: 103;
SEQ ID NO: 104 and SEQ ID NO: 105;
SEQ ID NO: 106 and SEQ ID NO: 107;
SEQ ID NO: 108 and SEQ ID NO: 109;
SEQ ID NO: 110 and SEQ ID NO: 111;
SEQ ID NO: 112 and SEQ ID NO: 113;
SEQ ID NO: 114 and SEQ ID NO: 115;
SEQ ID NO: 116 and SEQ ID NO: 117;
SEQ ID NO: 118 and SEQ ID NO: 119;
SEQ ID NO: 120 and SEQ ID NO: 121;
SEQ ID NO: 122 and SEQ ID NO: 123;
SEQ ID NO: 124 and SEQ ID NO: 125;
SEQ ID NO: 126 and SEQ ID NO: 127;
SEQ ID NO: 128 and SEQ ID NO: 129;
SEQ ID NO: 130 and SEQ ID NO: 131;
SEQ ID NO: 132 and SEQ ID NO: 133;
SEQ ID NO: 134 and SEQ ID NO: 135;
SEQ ID NO: 136 and SEQ ID NO: 137;
SEQ ID NO: 138 and SEQ ID NO: 139; and
SEQ ID NO: 140 and SEQ ID NO: 141;
(b) the nucleic acid sequences in (a) wherein T can also be U;
(c) nucleic acid sequences complementary to (a) or (b); and
(d) nucleic acid sequences which are homologous to (a), (b) or (c).

77. Isolated nucleic acid molecules prepared using PCR and the pairs of primers according to claim 76.


SEQUENCE LISTING (141 sequences)

SEQ ID NO: 1 - DNA, 598 bp, Homo sapiens
ccctgtggaatgtacctatgtgagtttcagaaattctcaaaatacgtgttcaaaaatttctgcttttgcatctttgggacacctcagaaaacttattaacaactgtgaatatgagaaatacagaagaaaataataagccctctatacataaatgcccagcacaattcattgttaaaaaacaaccaaacctcacactactgtatttcattatctgtactgaaagcaaatgctttgtgactattaaatgttgcacatcattcattcactgtatagtaatcattgactaaagccatttgtctgtgttttcttcttgtggttgtatatatcaggtaaaatattttccaaagagccatgtgtcatgtaatactgaaccactttgatattgagacattaatttgtacccttgttattatctactagtaataatgtaatactgtagaaatattgctctaattcttttcaaaattgttgcatcccccttagaatgtttctatttccataaggatttaggtatgctattatcccttcttataccctaagatgaagctgtttttgtgctctttgttcatcattggccctcattccaagcactttacgc

SEQ ID NO: 2 - DNA, 568 bp, Homo sapiens
ccttcaacacggacacgctctgctgaccacccagagctgggccagggaggacacgctccactgaccacccagagctgggccagggactcaacaatggggacagaagtcccccagtgcctgccagggcctggagtggaggttcaggaccaaggggcttctggtcctccagcccctgtactcggccatgccctgcggtcactgcggttgccgcccctaattgtgccaaaggctgacccggcccgggctgcgtacacccttgccctgctttgccttaaagcctcggggtctgcccggcccctcgcccctgcctggcactgctcaccgcccaaggcgacgccggctggaccaggcactgctggcctttctcctgcccggcctcggaaccagcttttctctcttacgatgaaggctgatgccgagagcgggctgtgggcggagctgggtcagtcccgtatttattttgctttgagaagaggctcctctggccctgctctcctgcagggaggtggctgtcccgcgggaagccatcagcttgggccagctggcaggtggcaggaatggagaagct

SEQ ID NO: 3 - DNA, 628 bp, Homo sapiens
aaggaaaagtacggcgtggacgactactccgtgagccagatctcgctggaacaggtcttcctgagcttcgcccacctgcagccgcccaccgcagaggaggggcgatgaggggtggcggctgtctcgccatcaggcagggacaggacgggcaagcagggcccatcttacatcctctctctccaagtttatctcatcctttatttttaatcacttttttctatgatggatatgaaaaattcaaggcagtatgcacagaatggacgagtgcagcccagccctcatgcccaggatcagcatgcgcatctccatgtctgcatactctggagttcactttcccagagctggggcaggccgggcagtctgcgggcaagctccggggtctctgggtggagagctgacccaggaagggctgcagctgagctgggggttgaatttctccaggcactccctggagagaggacccagtgacttgtccaagtttacacacgacactaatctcccctggggaggaagcgggaagccagccaggttgaactgtagcgaggcccccaggccgccaggaatggaccatgcagatcactgtcagtggagggaagctgctgactgtgattaggtgctggggtcttag

SEQ ID NO: 4 - DNA, 745 bp, Homo sapiens
gagcatcatcagaaaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactgatctttcacaccgctcgttcctgcagccagaaaggaactctgggcagctggaggcgcaggagcctgtgcccatatggtcatccaaatggactggcccagcgtaaatgaccccactgcagcagaaaacaaacacacgaggagcatgcagcgaattcagaaagaggtctttcagaaggaaaccgaaactgacttgctcacctggaacacctgatggtgaaaccaaacaaatacaaaatccttctccagaccccagaactagaaaccc

SEQ ID NO: 5 - DNA, 772 bp, Homo sapiens
aatgcaagccgtcaggaaagtttttcttctattttggcttataaaattcctaaggaagatgttcagtccctttcacaatctttttttaagctggaagaagctaaacatgcttttgccattgaagaatatagcttttctcaagcaacattggaacaggtttttgtagaactcactaaagaacaagaggaggaagataatagttgtggaactttaaacagcacactttggtgggaacgaacacaagaagatagagtagtattttgaatttgtattgttcggtctgcttactgggacttctttctttttcacttaattttaactttggtttaaaaagttttttattggaatggtaactggagaaccaagaacgcacttgaaatttttctaagctccttaattgaaatgctgtggttgtgtgttttgcttttctttaaataaaacgtatgtataattaagtgaagctgcatgtttgtattgaagtatattgaactatatagtttgtatgtcatctttttcaccattcagaaacagtgcttctgaatttgtgatttaaaggaattgtaatagaatagttttatttttaagttatctttaagtttatgccatcttcttaaataagtacgtaatgttccaatctaaataaaaaactaatacataactaatgcatagaaaagatacataaagcaatgtgaaagtttcttgcttctcctttttaatttctaaaaaagccactttgaatggaagttgtcatccgtaaaagctgaagtgtaag

SEQ ID NO: 6 - DNA, 831 bp, Homo sapiens
agttgtgttttgtgctgagcctcctgggaaactcacctgtcttgctcctggatgaaccatctacgggcatagaccccacagggcagcagcaaatgtggcaggcaatccaggcagtcgttaaaaacacagagagaggtgtcctcctgaccacccataacctggctgaggcggaagccttgtgtgaccgtgtggccatcatggtgtctggaaggcttagatgcattggctccatccaacacctgaaaaacaaacttggcaaggattacattctagagctaaaagtgaaggaaacgtctcaagtgactttggtccacactgagattctgaagcttttcccacaggctgcagggcaggaaaggtattcctctttgttaacctataagctgcccgtggcagacgtttaccctctatcacagacctttcacaaattagaagcagtgaagcataactttaacctggaagaatacagcctttctcagtgcacactggagaaggtattcttagagctttctaaagaacaggaagtaggaaattttgatgaagaaattgatacaacaatgagatggaaactcctccctcattcagatgaaccttaaaacctcaaacctagtaattttttgttgatctcctataaacttatgttttatgtaataattaatagtatgtttaattttaaagatcatttaaaattaacatcaggtatattttgtaaatttagttaacaaatacataaattttaaaattattcttcctctcaaacataggggtgatagcaaacctgtgataaaggcaatacaaaatattagtaaagtcacccaaagagtcaggcac

SEQ ID NO: 7 - DNA, 641 bp, Homo sapiens
atagcatggaggagtgtgaagcgctctgctcgcgcctagccatcatggtgaatgggcggttccgctgcctgggcagcccgcaacatctcaagggcagattcgcggcgggtcacacactgaccctgcgggtgcccgccgcaaggtcccagccggcagcggccttcgtggcggccgagttccctgggtcggagctgcgcgaggcacatggaggccgcctgcgcttccagctgccgccgggagggcgctgcgccctggcgcgcgtctttggagagctggcggtgcacggcgcagagcacggcgtggaggacttttccgtgagccagacgatgctggaggaggtattcttgtacttctccaaggaccaggggaaggacgaggacaccgaagagcagaaggaggcaggagtgggagtggaccccgcgccaggcctgcagcaccccaaacgcgtcagccagttcctcgatgaccctagcactgccgagactgtgctctgagcctccctcccctgcggggccgcggggaggccctgggaatggcaagggcaaggtagagtgcctaggagccctggactcaggctggcagaggggctggtgccctggagaaaataaagagaaggctggagagaagccgtggtggtgaaa

SEQ ID NO: 8 - DNA, 707 bp, Homo sapiens
gctgggtgattttgaggaggattttgatccctcagtgaagtggaagctcctcccccaggaagagccttaaaaccccaaattctgtgttcctgtttaaacccgtggttttttttaaatacatttatttttatagcagcaatgttctatttttagaaactatattataagtacagaaatggttctccgtgtggtgggaggaggaggttcgggtgctgggtaagtgccatgtcagtgtggacagaggcatttgactaagccaacctcctctcacagcctctgtatctctgcaggccatactggttccattgttctgtataatactgaataaataaatttacttttacatgatcgtataagtttctagataagataaacaaattctgtttaaatttttttaataaaaatcttaaaacactttttttctaacctagactgagaaattcatgtttacttttctaggtgtatgatactttgtaaagttgatactttcctaagaatttaacatgtcatatttttgaaatagatttaagtgtgcttcttattgctaaaaatactaaatgtcatgggtcatagtatctgatatcaatatcgttgataacatatccacaggtaacaccatgatgtaggcataaatggaaaacaaaaaccctactatttcaaatatattgtacttttttatttctgtaagccaactgtgtgccattttc

SEQ ID NO: 9 - DNA, 722 bp, Homo sapiens
tgtgccagcaaccaaatcccatgtttcctactgtgttaagtttaaaaatgcatttattatagaattgtctacatttctgaggatgtcatggagaatgcttaattttctttctctgaacttcaaaatattaaatattttcttatttttttgattaaagtataaattaagacaccctattgacttccgggtaaggggagtcaattgattacccagcagcacagtatttgctttttataattccctttttaaatacttgttcttaattgactggttttccttttctgtcatttttcagagtttagattgtgagtccatgttttgtctgttgtgcctataaaggaaatttgaaatctgtatcattctactataaagacacatgcacacgtatgtttattgcagcactgtttacaatagcaaagacttggaaccaaccaaaatacccacaaatgatagaccggataaagaaaacgtgacacatatacaccatggaatactatgcagccatagaaaaggatgagttcatattcttcacagggacatggatgaagctggaaaccatcatcctcagcaaactaacacaggaacagaaaaccaaacaccgcatgttctcactcataagtgggaattgaacaatgagaatacatggacacagggaggggaacaccacaccctggggcctgttggggggatgggggctaggggagggatagcattaggagaaa

SEQ ID NO: 10 - DNA, 523 bp, Homo sapiens
aggagctgggaaatgttgatgataaaattgatacaacagttgaatggaaacttctcccacaggaagacccttaaaatgaagaacctcctaacattcaattttaggtcctactacattgttagtttccataattctacaagaatgtttccttttacttcagttaacaaaagaaaacatttaataaacattcaataatgattacagttttcatttttaaaaatttaggatgaaggaaacaaggaaatatagggaaaagtagtagacaaaattaacaaaatcagacatgttattcatccccaacatgggtctattttgtgcttaaaaataatttaaaaatcatacaatattaggttggttttcggttattatcaataaagctaacactgagaacattttacaaataaaaatatgagttttttagcctgaacttcaaatgtatcagctatttttaaacattatttactcggattctaatttaatgtgacattgactataagaaggtctgataaactgatgaaatggc

SEQ ID NO: 11 - DNA, 764 bp, Homo sapiens
cctgctggagagtgttttgggcttcttggagtgaatggagcaggaaagaccactatattcaagatgctgacaggagacatcattccttcaagtggaaacattctgatcagaaataagaccggatctctgggtcacgttgattctcacagctcattagttggctactgtcctcaggaagatgccttagatgacctggtaactgtggaagaacatttgtatttctatgccagggtacatggaattccagaaaaggatattaaagaaactgttcataaactccttaggagacttcacctgatgcccttcaaggacagagctacctctatgtgcagttatggcacaaaaagaaaattatccactgcactggccttgatagggaaaccttccattctactgctggatgagccgagctctggcatggatccgaagtcgaaacggcacctctggaagatcatttcagaagaagtacagaacaaatgttccgtcatcctcacatctcacagcatggaagaatgtgaagctctctgtaccaggttggccattatggtgaatggaaagtttcaatgtattggatctttgcagcacataaagagcaggtttggacgaggatttactgtcaaagttcacttgaagaataacaaagtgaccatggagaccctcacaaagttcatgcagctgcactttccaaaaacatacttaaaagatcagcacctcagcatgctagagtatcatgtaccagtcacagcaggaggagtcgcaaacat

SEQ ID NO: 12 - DNA, 790 bp, Homo sapiens
catcctgtttgactgcagcattgctgagaacattgcctatggagacaacagccgggtggtgtcacaggaagagattgtgagggcagcaaaggaggccaacatacatgccttcatcgagtcactgcctaataaatatagcactaaagtaggagacaaaggaactcagctctctggtggccagaaacaacgcattgccatagctcgtgcccttgttagacagcctcatattttgcttttggatgaagccacgtcagctctggatacagaaagtgaaaaggttgtccaagaagccctggacaaagccagagaaggccgcacctgcattgtgattgctcaccgcctgtccaccatccagaatgcagacttaatagtggtgtttcagaatggcagagtcaaggagcatggcacgcatcagcagctgctggcacagaaaggcatctatttttcaatggtcagtgtccaggctggaacaaagcgccagtgaactctgactgtatgagatgttaaatactttttaatatttgtttagatatgacatttattcaaagttaaaagcaaacacttacagaattatgaagaggtatctgtttaacatttcctcagtcaagttcagagtcttcagagacttcgtaattaaaggaacagagtgagagacatcatcaagtggagagaaatcatagtttaaactgcattataaattttataacagaattaaagtagattttaaaagataaaatgtgtaattttgtttatattttcccatttggactgtaactgactgccttgctaa

SEQ ID NO: 13 - DNA, 709 bp, Homo sapiens
atattgcctatggcctgacccagaagccaactatggaggaaatcacagctgctgcagtaaagtctggggcccatagtttcatctctggactccctcagggctatgacacagaggtagacgaggctgggagccagctgtcagggggtcagcgacaggcagtggcgttggcccgagcattgatccggaaaccgtgtgtacttatcctggatgatgccaccagtgccctggatgcaaacagccagttacaggtggagcagctcctgtacgaaagccctgagcggtactcccgctcagtgcttctcatcacccagcacctcagcctggtggagcaggctgaccacatcctctttctggaaggaggcgctatccgggaggggggaacccaccagcagctcatggagaaaaaggggtgctactgggccatggtgcaggctcctgcagatgctccagaatgaaagccttctcagacctgcgcactccatctccctcccttttcttctctctgtggtggagaaccacagctgcagagtagcagctgcctccaggatgagttacttgaaatttgccttgagtgtgttacctcctttccaagctcctcgtgataatgcagacttcctggagtacaaacacaggatttgtaattcctactgtaacggagtttagagccagggctgatgctttggtgtggccagcactctgaaactgagaa

SEQ ID NO: 14 - DNA, 817 bp, Homo sapiens
gggagtaggagctatgctaagtgtttttcatgtattatcgtttttaatcattatccccaaccctatgaggttggttattatccccattttacagatgaggaaactgaagctcaaagaggctcaatgactttcccaaggtggtcgtagtggtggagttggagtttgaacacaggcctgaccctagagtccacaccctgacccaatcaattatattgcatcttgggtccataaaccctaatccataatcccatcaagaaaagctctgctgctcttagctctaaataattcagaatctattctcttctctccagtcccgttgttatagtcttcactcatagacttaagatgatcccatcaccagagaggtttctctaccattagcttccctcttccggccattcttcacaaagtcatttttctaaattctgtgtcacatacgatgatggcatttctggaaattccttcaggtgctctcaagccctgctgcagagatccttttcagagcacacactgttccagcccatctgtctcaccctctcctgttgtatccagctccacgacaaactttctgccttccccaacacctttgtgcctttgcatatggtgttttcttgcccattttctgctcgactcgcccctgattttcaagttcaagacttaactcagggttcaggtcttccaggaggccttacttatgtcgtcagtctggggaactctccatgtgcttctatcactgtgcggttacctctttcacagcccttttaaagttctatcttccctttcccaccttttttgaccttccactagaccatgagca

SEQ ID NO: 15 - DNA, 790 bp, Homo sapiens
ttgacagctacagtgaagaggggctgaagcctgataaatttgaaggaaatataacatttaatgaagtcgtgttcaactatcccacccgagcaaacgtgccagtgcttcaggggctgagcctggaggtgaagaaaggccagacactagccctggtgggcagcagtggctgtgggaagagcacggtggtccagctcctggagcggttctacgaccccttggcggggacagtgcttctcgatggtcaagaagcaaagaaactcaatgtccagtggctcagagctcaactcggaatcgtgtctcaggagcctatcctatttgactgcagcattgccgagaatattgcctatggagacaacagccgggttgtatcacaggatgaaattgtgagtgcagccaaagctgccaacatacatcctttcatcgagacgttaccccacaaatatgaaacaagagtgggagataaggggactcagctctcaggaggtcaaaaacagaggattgctattgcccgagccctcatcagacaacctcaaatcctcctgttggatgaagctacatcagctctggatactgaaagtgaaaaggttgtccaagaagccctggacaaagccagagaaggccgcacctgcattgtgattgctcaccgcctgtccaccatccagaatgcagacttaatagtggtgtttcagaatgggagagtcaaggagcatggcacgcatcagcagctgctggcacagaaaggcatctatttttcaatggtcagtgtccaggctgggacacagaacttatg

SEQ ID NO: 16 - DNA, 705 bp, Homo sapiens
ttcgcttctacgacatcagctctggctgcatccgaatagatgggcaggacatttcacaggtgacccaggcctctctccggtctcacattggagttgtgccccaagacactgtcctctttaatgacaccatcgccgacaatatccgttacggccgtgtcacagctgggaatgatgaggtggaggctgctgctcaggctgcaggcatccatgatgccattatggctttccctgaagggtacaggacacaggtgggcgagcggggactgaagctgagcggcggggagaagcagcgcgtcgccattgcccgcaccatcctcaaggctccgggcatcattctgctggatgaggcaacgtcagcgctggatacatctaatgagagggccatccaggcttctctggccaaagtctgtgccaaccgcaccaccatcgtagtggcacacaggctctcaactgtggtcaatgctgaccagatcctcgtcatcaaggatggctgcatcgtggagaggggacgacacgaggctctgttgtcccgaggtggggtgtatgctgacatgtggcagctgcagcagggacaggaagaaacctctgaagacactaagcctcagaccatggaacggtgacaaaagtttggccacttccctctcaaagactaacccagaagggaataagatgtgtctcctttccctggcttatttcatcctggtc

SEQ ID NO: 17 - DNA, 776 bp, Homo sapiens
ccctgcaggaaagaaagtggccattgtaggaggtagtgggtcagggaaaagcacaatagtgaggctattatttcgcttctatgagcctcaaaagggtagcatttatcttgctggtcaaaatatacaagatgtgagcctggaaagccttcggagggcagtgggagtggtacctcaggatgctgtcctcttccataatactatttattacaacctcttatatggaaacatcagtgcttcacctgaggaagtgtatgcagtggcaaaattagctggacttcatgatgcaattcttcgaatgccacatggatatgacacccaagtaggggaacgaggactcaagctttcaggaggagaaaagcaaagagtagcaattgcaagagccattttgaaggaccccccagtcatactctacgatgaagctacttcatcgttagattcgattactgaagagactattcttggtgccatgaaggatgtggtcaaacacagaacttctattttcattgcacacagattgtcaacagtggttgatgcagatgaaatcattgtcttggatcagggtaaggtagccgaacgtggtacccaccatggtttgcttgctaaccctcatagtatctattcagaaatgtggcatacacagagcagccgtgtgcagaaccatgataaccccaaatgggaagcaaagaaagaaaatatatccaaagaggaggaaagaaagaaactacaagaagaaattgtcaatagtgtgaaaggctgtggaaactgttcgtgctaag

SEQ ID NO: 18 - DNA, 702 bp, Homo sapiens
aggttgtcggtttcatcagccaggagcccgtcctgtttgggacgaccatcatggaaaacatccgctttgggaagctggaagcttccgatgaagaggtgtacacagccgcccgggaagcgaatgctcacgagttcatcaccagtttccccgagggctacaacacggtcgtcggtgaacggggcactaccctgtctgggggccagaagcagcgcctggccatcgcccgagcccttatcaagcagcccacggtgctgatactggatgaagctaccagcgcgctggatgcagagtccgagcgggttgtacaggaggccctggaccgggccagtgcaggccgcacggtgctggtaattgcccaccggctcagcactgtccgtggggcccactgcattgtcgtcatggccgatggccgtgtctgggaggctgggacacatgaagagctcctgaagaaaggcgggctatacgccgagctcatccggaggcaggccctggatgccccgaggacagcggccccaccgcccaaaaagccagaaggccccaggagccaccagcacaagtcctgagaagggccccctgaggtgtggtcgctgccaagcatcagtgttagggctggggctcagcctgggggagcctactggggactgagcccccaggagggccagcatgtggagagtcgctgcggctgctcctgctcacaataaa

SEQ ID NO: 19 - DNA, 706 bp, Homo sapiens
tggatcaccgcttcctgcatcttgcccctggtccctgccccattcccagggcactccttacccctgctgccctgagccaacgccttcacggacctccctagcctcctaagcaaaggtagagctgcctttttaaacctaggtcttaccagggtttttactgtttggtttgaggcaccccagtcaactcctagatttcaaaaacctttttctaattgggagtaatggcgggcactttcaccaagatgttctagaaacttctgagccaggagtgaatggcccttccttagtagcctgggggatgtccagagactaggcctctcccctttacccctccagagaaggggcttccctgtcccggagggacacggggaacgggattttccgtctctccctcttgccagctctgtgagtctggccagggcgggtagggagcgtggagggcatctgtctgccatcgcccgctgccaatctaagccagtctcactgtgaaccacacgaaacctcaactgggggagtgaggggctggccaggtctggaggggcctcaggggtgcccagcccggcacccagcgctttcgcccctcgtccacccacccctggctggcagcctccctccccacacccgcccctgtgctctgctgtctggaggccacgtggatgttcatgagatgcattctcttctgtctttggtggatgggatggtggca

SEQ ID NO: 20 - DNA, 538 bp, Homo sapiens
gcaaggcatgaactgctaggtattattaagaatgaatgatttttgcatttaagttgtttgaaggcatgtattttgaaaaatatctgttacaaatttataatttcaagacaaattgaatcttattttataatacttttggaatttcattaataaggctaaaatttgaggaatataactaattttcagccttaagacatttaagtttggaagtccttgctattcaacagaataacaagaaaacttcagaatgtatcactctcctgaaaagaagatattaataagcccttttatttatggttatagttttatttatagtctcaaaattcctaaagcaatgctacaaccattgaatttgccatattttgtatcagtgctgttaatttgctgttgcctcaagaaaaagtgctttttctccatggatgaggctagaccctaagaagtaattaagtcaatgtaaatcaaatggaagttttcccatgaactaagaatttattagttccctgattagactggaagaagaaaccactatttcatgaaa

SEQ ID NO: 21 - DNA, 753 bp, Homo sapiens
ttgtcattgcccatcgcttgtccaccatccagaacgcggatatcattgctgtcatggcacagggggtggtgattgaaaaggggacccatgaagaactgatggcccaaaaaggagcctactacaaactagtcaccactggatcccccatcagttgacccaatgcaagaatctcagacacacatgacgcaccagttacaggggttgtttttaaagaaaaaaacaatcccagcaggagggattgctgggattgttttttctttaaagaagaatgttaatattttacttttacagtcattttcctacatcggaatccaagctaatttctaatggccttccataataattctgctttagatgtgtatacagaaaatgaaagaaactagggtccatatgagggaaaacccaatgtcaagtggcagctcagccaccactcagtgcttctctgtgcaggagccagtcctgattaatatgtgggaattagtgagacatcagggagtaagtgacactttgaactcctcaagggcagagaactgtctttcatttttgaaccctcggtgtacacagaggcgggtctataacaggcaatcaacaaacgtttcttgagctagaccaaggtcagatttgaaaagaacagaaggactgaagaccagctgtgtttcttaactaaatttgtctttcaagtgaaaccagcttccttcatctctaaggctaaggatagggaaagggtggatgctctcaggctgagggaggcag

SEQ ID NO: 22 - DNA, 660 bp, Homo sapiens
gctcccatcacctctaacatccttgtctgggtctaccaggaacgcttcatttccttggggctgcagttttgtggttgaggggcctggagaaaatcattttctccccttggcagtgtcccagggccctggatggtcctcttaccaacatctggtcttccaggcactcaaaagctgggaaccagcatctcagcgccagctctaccagttctcgttttgggccagaggcagcctctgcactcccacgcctgtcctcctggaagggacctggttggactaacggctaacctggacctggaactgtagggccaggggattgtctcagggccgacgttccacctggggcttccctccccacccaccccgactccaggctttcccttttttcttttgttcaacattgtaagaacaatcaatgctgttattactgatcccaccatgattgatgtggggtaaatattaaggagatggcctcatgggaatttgaccttgactagaaatagagactgagagtgagcaaccagctggaaggtactatgccagtcctagcagaaaaatgtgttaggggcctggcccaaagcagtgttggttgcttacagtgttgattgattttgttcttttttcttaccacctcttttctttccctctcatggtacctgctca

SEQ ID NO: 23 - DNA, 810 bp, Homo sapiens
gtagcatggagaagattggtgtggtgggcaggacaggagctggaaagtcatccctcacaaactgcctcttcagaatcttagaggctgccggtggtcagattatcattgatggagtagatattgcttccattgggctccacgacctccgagagaagctgaccatcatcccccaggaccccatcctgttctctggaagcctgaggatgaatctcgaccctttcaacaactactcagatgaggagatttggaaggccttggagctggctcacctcaagtcttttgtggccagcctgcaacttgggttatcccacgaagtgacagaggctggtggcaacctgagcataggccagaggcagctgctgtgcctgggcagggctctgcttcggaaatccaagatcctggtcctggatgaggccactgctgcggtggatctagagacagacaacctcattcagacgaccatccaaaacgagttcgcccactgcacagtgatcaccatcgcccacaggctgcacaccatcatggacagtgacaaggtaatggtcctagacaacgggaagattatagagtgcggcagccctgaagaactgctacaaatccctggacccttttactttatggctaaggaagctggcattgagaatgtgaacagcacaaaattctagcagaaggccccatgggttagaaaaggactataagaataatttcttatttaattttattttttataaaatacagaatacatacaaaagtgtgtataaaatgtacgttttaaaaaaggataagtgaacacccatgaacctactaccc

SEQ ID NO: 24 - DNA, 722 bp, Homo sapiens
caagagccgcatcctggttttagacgaggccacagctgccatcgacctggagactgacaacctcatccaggctaccatccgcacccagtttgatacctgcactgtcctgaccatcgcacaccggcttaacactatcatggactacaccagggtcctggtcctggacaaaggagtagtagctgaatttgattctccagccaacctcattgcagctagaggcatcttctacgggatggccagagatgctggacttgcctaaaatatattcctgagatttcctcctggcctttcctggttttcatcaggaaggaaatgacaccaaatatgtccgcagaatggacttgatagcaaacactgggggcaccttaagattttgcacctgtaaagtgccttacagggtaactgtgctgaatgctttagatgaggaaatgatccccaagtggtgaatgacacgcctaaggtcacagctagtttgagccagttagactagtcccccggtctcccgattcccaactgagtgttatttgcacactgcactgttttcaaataacgattttatgaaatgacctctgtcctccctctgatttttcatattttcctaaagtttcgtttctgttttttaataaaaagctttttcctcctggaacagaagacagctgctgggtcaggccacccctaggaactcagtcctgtactctggggtgctgcctgaatccattaaa

SEQ ID NO: 25 - DNA, 794 bp, Homo sapiens
tgggaagaaccggagctggaaaaagttccctcatctcagccctttttagattgtcagaacccgaaggtaaaatttggattgataagatcttgacaactgaaattggacttcacgatttaaggaagaaaatgtcaatcatacctcaggaacctgttttgttcactggaacaatgaggaaaaacctggatccctttaaggagcacacggatgaggaactgtggaatgccttacaagaggtacaacttaaagaaaccattgaagatcttcctggtaaaatggatactgaattagcagaatcaggatccaattttagtgttggacaaagacaactggtgtgccttgccagggcaattctcaggaaaaatcagatattgattattgatgaagcgacggcaaatgtggatccaagaactgatgagttaatacaaaaaaaaatccgggagaaatttgcccactgcaccgtgctaaccattgcacacagattgaacaccattattgacagcgacaagataatggttttagattcaggaagactgaaagaatatgatgagccgtatgttttgctgcaaaataaagagagcctattttacaagatggtgcaacaactgggcaaggcagaagccgctgccctcactgaaacagcaaaacaggtatacttcaaaagaaattatccacatattggtcacactgaccacatggttacaaacacttccaatggacagccctcgaccttaactattttcgagacagcactgtgaatccaaccaaaatgtcaagtccgttccgaaggcatt

SEQ ID NO: 26 - DNA, 646 bp, Homo sapiens
aaggaagacgtgtggcaatagtgggccctccgacagccccctctgccgcctccccacagccgctccaggggtggctggagacgggtgggcggctggagaccatgcagagcgccgtgagttctcagggctcctgccttctgtcctggtgtcacttactgtttctgtcaggagagcagcggggcgaagcccaggccccttttcactccctccatcaagaatggggatcacagagacattcctccgagccggggagtttctttcctgccttcttctttttgctgttgtttctaaacaagaatcagtctatccacagagagtcccactgcctcaggttcctatggctggccactgcacagagctctccagctccaagacctgttggttccaagccctggagccaactgctgctttttgaggtggcactttttcatttgcctattcccacacctccacagttcagtggcagggctcaggatttcgtgggtctgttttcctttctcaccgcagtcgtcgcacagtctctctctctctctcccctcaaagtctgcaactttaagcagctcttgctaatcagtgtctcacactggcgtagaagtttttgtactgtaaagagacctacctcaggttgctggttgctgtgtggttt

SEQ ID NO: 27 - DNA, 747 bp, Homo sapiens
tcgtgtcagtggagcggatgcaggactatgcctggacgcccaaggaggctccctggaggctgcccacatgtgcagctcagcccccctggcctcagggcgggcagatcgagttccgggactttgggctaagataccgacctgagctcccgctggctgtgcagggcgtgtccttcaagatccacgcaggagagaaggtgggcatcgttggcaggaccggggcagggaagtcctccctggccagtgggctgctgcggctccaggaggcagctgagggtgggatctggatcgacggggtccccattgcccacgtggggctgcacacactgcgctccaggatcagcatcatcccccaggaccccatcctgttccctggctctctgcggatgaacctcgacctgctgcaggagcactcggacgaggctatctgggcagccctggagacggtgcagctcaaagccttggtggccagcctgcccggccagctgcagtacaagtgtgctgaccgaggcgaggacctgagcgtgggccagaaacagctcctgtgtctggcacgtgcccttctccggaagacccagatcctcatcctggacgaggctactgctgccgtggaccctggcacggagctgcagatgcaggccatgctcgggagctggtttgcacagtgcactgtgctgctcattgcccaccgcctgcgctccgtgatggactgtgcccgggttctggtcatggacaaggggcaggtggcag

SEQ ID NO: 28 - DNA, 748 bp, Homo sapiens
tctttcacaggggacaggatggttcccttgatgaagaagttgatatgccttttcccaactccagaaagtgacaagctcacagacctttgaactagagtttagctggaaaagtatgttagtgcaaattgtcacaggacagcccttctttccacagaagctccaggtagagggtgtgtaagtagataggccatgggcactgtgggtagacacacatgaagtccaagcatttagatgtataggttgatggtggtatgttttcaggctagatgtatgtacttcatgctgtctacactaagagagaatgagagacacactgaagaagcaccaatcatgaattagttttatatgcttctgttttataattttgtgaagcaaaattttttctctaggaaatatttattttaataatgtttcaaacatatattacaatgctgtattttaaaagaatgattatgaattacatttgtataaaataatttttatatttgaaatattgactttttatggcactagtatttttatgaaatattatgttaaaactgggacaggggagaacctagggtgatattaaccaggggccatgaatcaccttttggtctggagggaagccttggggctgatcgagttgttgcccacagctgtatgattcccagccagacacagcctcttagatgcagttctgaagaagatggtaccaccagtctgactgtttccatcaagggtacactgccttctcaactccaaactg

SEQ ID NO: 29 - DNA, 805 bp, Homo sapiens
aaaccgaggcagagagctacgaggggctcctggcaccatcgctgatcccaaagaactggccagaccaagggaagatccagatccagaacctgagcgtgcgctacgacagctccctgaagccggtgctgaagcacgtcaatgccctcatctcccctggacagaagatcgggatctgcggccgcaccggcagtgggaagtcctccttctctcttgccttcttccgcatggtggacacgttcgaagggcacatcatcattgatggcattgacatccgcaaactgccgctgcacaccctgccgtcacgcctctccatcatcctgcaggaccccgtcctcttcagcggcaccatccgatttaacctggaccctgagaggaagtgctcagatagcacactgtgggaggccctggaaatcgcccagctgaagctggtggtgaaggcactgccaggaggcctcgatgccatcatcacagaaggcggggagaatttcagccagggacagaggcagctgttctgcctggcccgggccttcgtgaggaagaccagcatcttcatcatggacgaggccacggcttccattgacatggccacggaaaacatcctccaaaaggtggtgatgacagccttcgcagaccgcactgtggtcaccatcgcgcatcgagtgcacaccatcctgagtgcagacctggtgatcgtcctgaagcggggtgccatccttgagttcgataagccagagaagctgctcagccggaaggacagcgtcttcgcctccttcgtccgtgcagacaagtgacctgccagagccca

SEQ ID NO: 30 - DNA, 782 bp, Homo sapiens
tgggtgcagtgaagaaggtgaacagtttcctgactatggagtcagagaactatgaaggcacaatggatccttctcaagttccagaacattggccacaagaaggggagatcaagatacatgatctgtgtgtcagatatgaaaataatctgaaacctgttcttaagcacgtcaaggcttacatcaaacctggacaaaaggtgggcatatgtggtcgcactggcagtgggaaatcatcgttatctctggctttcttcagaatggttgatatatttgatggaaaaattgtcattgatgggatagacatttccaaattaccactgcacacactacgttctagactttcaatcattctgcaggatccaatactattcagtggttccattagatttaatttagatccagagtgcaaatgcacagatgacagactctgggaagccttagaaattgctcagctgaagaatatggtcaaatctctacctggaggtctagatgcggttgtcactgaaggtggggagaattttagcgtgggacagagacagctattttgccttgccagggcctttgtccgcaaaagcagcattcttattatggatgaggcaacagcttccattgacatggccacagagaatattttgcaaaaagtagtaatgacagcctttgcagaccggaccgtggtgacaatggctcaccgtgtctcttctattatggatgcaggccttgttttagtcttttctgagggtattttagtggagtgtgatactgtcccaaatttgttcgcccac

SEQ ID NO: 31 - DNA, 892 bp, Homo sapiens
tcttccctgttgttggtgctcttccggctgctagagcccagttcagggcgagtgctgctggacggcgtggacaccagccagctggagctggcccagctcagatcccagttggctatcatcccccaggagccctttttgttcagtgggactgttcgggaaaacctggacccccagggcctacataaggacagggccttgtggcaggccctgaagcagtgccacctgagtgaggtgattacatccatgggtggtctggatggtgagctgggtgaggggggccggagcttatctcttgggcagaggcagctgttgtgtttggccagggctctcctcacagatgccaagatcctgtgtatcgatgaggccacagcaagtgtggaccagaagacagaccagctgctccagcagaccatctgcaaacgctttgccaacaagacagtgctgaccattgcccataggctcaacacgatcctgaactcagaccgggtgctggtgctacaagcggggagagtggtagagctggactccccggccaccctgcgcaaccagccccactccctgttccagcagctgctgcagagcagccagcagggagtccctgcctcactcggaggtccctgagcccaatcccacaccctgcagagttctcccctctctctgatccaggccgggcctatacagaggtgctggctgcttgtttacattctcctctggggctctacctctccacacttccccagaagggaaaagggcaccctggattactctttggaaatcactccttggtgggcagcatcctgaggcttccccagaaccaggcctctgctctggccctcttgcatctggaacgccaggtgggtttttctggcataggagcccacttgcattttca

SEQ ID NO: 32 - DNA, 764 bp, Homo sapiens
gattctcattgacggcgtggacatttgcagcatcggcctggaggacttgcggtccaagctctcagtgatccctcaagatccagtgctgctctcaggaaccatcagattcaacctagatccctttgaccgtcacactgaccagcagatctgggatgccttggagaggacattcctgaccaaggccatctcaaagttccccaaaaagctgcatacagatgtggtggaaaacggtggaaacttctctgtgggggagaggcagctgctctgcattgccagggctgtgcttcgcaactccaagatcatccttatcgatgaagccacagcctccattgacatggagacagacaccctgatccagcgcacaatccgtgaagccttccagggctgcaccgtgctcgtcattgcccaccgtgtcaccactgtgctgaactgtgaccacatcctggttatgggcaatgggaaggtggtagaatttgatcggccggaggtactgcggaagaagcctgggtcattgttcgcagccctcatggccacagccacttcttcactgagataaggagatgtggagacttcatggaggctggcagctgagctcagaggttcacacaggtgcagcttcgaggcccacagtctgcgaccttcttgtttggagatgagaacttctcctggaagcaggggtaaatgtagggggggtggggattgctggatggaaaccctggaataggctacttgatggctctcaagaccttagaaccccagaacca

SEQ ID NO: 33 - DNA, 790 bp, Homo sapiens
ctggttatggaaaatgggaaggtgattgagtttgacaagcctgaagtccttgcagagaagccagattctgcatttgcgatgttactagcagcagaagtcagattgtagaggtcctggcggctgattctagaggaggaagaggctctgtgagatgaataggaggagtcttcaggaggaggggctgtcctctccgcaggcagccctggtcttcagcccctcccatccacggagtgagctggggctgaagttgtccccactgccatactcagtccatgtcaccccacttggtgggcttggggttggttctgggtggtgaaccggggcagacccagctaatggattaaaaaactgcccttcacctcccaaatccccaagggttcctcatgtgttttcaccaaaaccaccccagtgcctgagattgaaaatattgtaactttcagttagaaatcagccacaataaacaacatgggaaaatgccttaggatggagtttgcaaggtttccttgcccattatcagaaggaaaaagagcagaattttcttctcgtttaaccccactcacttccatcttgactgggtgacaagtggtaatgacacagatttgtagcgtgaaagactgaatacagtgtttggccaaaaatttttttaaaaatcatattatatgtttcaattgatctgttagaataaccaagaaaacaaaatgctggagtttctctataaatgacacttttatatcttctttattcgtcgttaaaacgcggtaggaaattaccctgaaatgtcgccttgcaa

SEQ ID NO: 34 - DNA, 787 bp, Homo sapiens
gcacctgtgggccatactaaaagatcccctacttacgttctggttgtcatgtttccctgtatttgataaaacacataattttgagaaaaataaagttttaaatgtatctatgtctcgacttttctgatgaagttataccagaaaagttaattatttgatgggcctgccatgtgaaaaccagaaaataacctcgtactcacaagccagtggaagggattcctgattttactaaaaaaaaaaaaaaaaaaagagagggcggggacaaatatcaaattaagcaagtaaagaaaaagaacaggtaagagtgtgtgtgtgtgtgtaacactttgacaatactaaactctcataagcatttaacacttcagatgtttaacatttctgcccctttctcaatttttatgacgtgcaggcaaattatcattttctgtgaacacagctcagattttggctggaatggctatggctatgcagtggcacttcttgttgtagtctttttgcaaactctgattcttcagcaatatcaacgttttaacatgctcacctcagcaaaagttaagacagctgtaaatggactgatctacaaaaaggccttacttttatcaaatgtttctcgacaaaagttttccactggggaaattattaacttgatgtcagcaactcatggacttgacagcaaacctcaatctcctctggtctgccccttttcaaatcctaatggccgtatatctcctttggcaagagctgggtccagcagtgttagcaggggtggcagtccttgtgtttgtta

SEQ ID NO: 35 - DNA, 488 bp, Homo sapiens
ttccctcctcgtcagtctctcaaagaccccatggtccatcccctgagggtggtcagccaaggctcccgttccgtgggatgccataaaagccgcccagtgggacccacagtcacacagagcgcctcacctgcatcctctcccccacaagagccccaaagatcccacgggagaggggagagggacgcacagcactgcctgccaagcgagaatgcaggccccgccccctcggcccctcaccacctctttctacagcctaatttattggattccctattcgtagccatctccgtggccaatgtgactaccgtgccagcagcgggggcggcccagcctctgagtcccgtggggccccggctcccaccggtgccaaacccagcccctgcggccgtcaccccgccagcctacactgccagccgccaccggggcacacgggcctctgcttgccagccaggagtgcggacaccatgttcccagctcagtgccaaaga

SEQ ID NO: 36 - DNA, 617 bp, Homo sapiens
gtggccaactaaacctgtacaaaatagctgacagttttataactaatttcaatataaaaattgttttaatggcatttgttgaaagaaaaaagcatggctaaaatgtatcaaatgccatatttttaaattttggactttaagcatcttaatgagggcatataacaaattaattttagtacaatcttaaatatttttaataaatcctttcattttaaaaagagaattgccaatacagaaaaggagtatccaaacaatgtctcaacctgataatttccttagcagaattacctattgcaacttctgttcagaaatacacagcttgtttttttgcccaaggatgagtctacattttaagaactgcaatggtataaaggaacttaaggattctgagaatcatagtaataacatacattggaatagtactttataatttacaatccccatttacatcatttcaccttaatgttgaggacaatgttttgaaacaaatactatttttcctactttgcttttgagaaaattgacactcagacttgccctaatcatgcactttacttaaggaaagatcgagaaatcaaatgaagttctcctgactctctggtttagtgctcttttgt

SEQ ID NO: 37 - DNA, 735 bp, Homo sapiens
tactcattccttgtgtgtgtcttggagtgcatttgactccaggaaaagccattttggttttccttaactaaatgataaatgtacccctctcagtctgcagtattgagttgtttaaagtatatgtgcagtcttgcttacaaggaggggttaccatgtatcacacctaatcttcccaatgtttgggatattaaaacacaaagtccttaacatgccaggctcaaggtcttataagagttctagatttttaagagaattagacaaatttgtgtgtgttagaagcccattcattagaagtgtggtggttatttggtattaaactccaaatgagccataggaaggcactacatgaaataatgcactgagtatgcaatgctatcactgtctttgactgtgattttatgtttaaaaagtatgttctaaaattattatatatacatgggtgaattatgtttccgaggcactgttttatctctgtgaatcttgaataacttttttatatttgggttatgatgtcaaacgatcctaagcgaagatgatttcagttcatcaaatcatcattaatgactttatgtattatttgcacagggagaattgaaactgagtataatcaataagctagatacgaaatcagtttctcaaactgagcttcagaaaggggcattttgtactcttgtttttgcataactggttttgtttttttgcagaattaactataacaatcactggctaccgaag

SEQ ID NO: 38 - DNA, 673 bp, Homo sapiens
ctccatatgcttgaagtgctgattacctacaaatgatttcagatcatgtttgctaaagagaaatctggaagtgtgagatctgtaagaaatgaaagaaatgactcttggagtcaagagatctggaaatcttttaatcagttaaattgtgcagcaatagatttttaactttaactgaccatttaagttttttaataagttttttacaaagaaaagttaaacattaaaaagaattacagctttctgtcttctctatcatggaatgattttttttattgaatctccagatttgtatttgacagcttggtgggaagggaagcacactctgctgttctggaatcttatgcccagggtttttcacttctccccacatctccctttccacttgccagtgttgtgtagttagaacctgaaccactaacttctaggggcctttggtctgccctaccttaacccaaatgaaagtaaatccctttccccttagccaaaataaggttgggttttctaaaaaaatagtctatattagggaacaacaacagcaaattagacaaaacccagaaagcacaaagcatgaggtggagttactgtgcccaaagtcctcactcagaccagtgcccctccagttcagttgtctatgtattaccttccttaccttcataatgtttgccaggcttct

SEQ ID NO: 39 - DNA, 756 bp, Homo sapiens
attccccgcaaaaaacccctaactttactctgaactttttttgtttttgcattccatgaggttctgtattcagtcattctctaggtaatgtcatttttgtacacatatatttatataatcactgattgagatttaggaaaaagcatttctaaagaatatttgcttcccttagaactacagactcgaaatctttaaagatggtgcctaagcatctatgtattttttttaagttccacagatttttctgttgggcaggccaaggattataaaccacttccctaaaggcaacattaatgcaaaagtccccagatggcaatacaaagtatcccctggtaccacatatattcatttgtgagtttggatatagagcacattatctaaaccattttgtagttccaaaaacccatctaaatttcttgagttcctgaattttgaacaggattacctggagcctggagccactttaagttgtacttctgactaaactggaattatgagtgaggaagagtgtttactaaataaatgactggggcaagcaaaattgaggaggaaattagaaactgtttgacaaactttaagagctacttgaaataacagaagtcttgattaatatgcaaataatggctagaaagtatggtttaactggaccctattatgccttttaaaaataatttcagtaacccataaatacatgttgtaaaaaattcaaatatacagaatggaataaaaaaatgatctccctttattaccctccca

SEQ ID NO: 40 - DNA, 591 bp, Homo sapiens
ttggaggccctgggtgaagtcatggtcagccggccccgagagtgaagctttccttcccagaagtctcccgagagacatatttgtgtggcctagaagtcctctgtggtctcccctcctctgaagactgcctctggcctgcagctgacctggcaaccattcaggcacatgaaggtggagtgtgaccttgatgtgaccgggatcccactctgattgcatccatttctctgaaagacttgtttgttctgcttctcttcatataactgagctggccttatccttggcatccccctaaacaaacaagaggtgaccaccttattgtgaggttccatccagccaagtttatgtggcctattgtctcaggactctcatcactcagaagcctgcctctgatttaccctacagcttcaggcccagctgccccccagtctttgggtggtgctgttcttttctggtggatttaatgctgactcactggtacaaacagctgttgaagctcagagctggaggtgagcttctgaggcctttgccattatccagcccaagatttggtgcctgcagcctcttgtctggttgaggacttggggcaggaaa

SEQ ID NO: 41 - DNA, 648 bp, Homo sapiens
tgctacccagagatcaaggagaaggaagaaatgaggaagatcattgggcgatacggtctcactgggaaacaacaggtgagcccaatccggaacttgtcagacgggcagaagtgccgagtgtgtctggcctggctggcctggcagaacccccacatgctcttcctggatgaacccaccaatcacctggatatcgagaccatcgacgccctggcagatgccatcaatgagtttgagggtggtatgatgctggtcagccatgacttcagactcattcagcaggttgcacaggaaatttgggtctgtgagaagcagacaatcaccaagtggcctggagacatcctggcttacaaggagcacctcaagtccaagctggtggatgaggagccccagctcaccaagaggacccacaacgtgtgcaccctgacattggcatctctgccaaggccatgagcatcatgaactcgtttgtaaacgacgtgtttgagcagctggcgtgtgaggctgcccggctggcccagtactcgggccggaccaccctgacatcccgagaagtccagacggctgtgcgtctgctgctgcctggggagctggccaagcacgctgtgtctgagggcaccaaggctgtcaccaagtacaccagctccaagt

SEQ ID NO: 42 - DNA, 719 bp, Homo sapiens
  misc_feature (45)..(45): n is a, c, g, or t
  misc_feature (251)..(251): n is a, c, g, or t
  misc_feature (255)..(255): n is a, c, g, or t
  misc_feature (504)..(504): n is a, c, g, or t
  misc_feature (513)..(513): n is a, c, g, or t
  misc_feature (643)..(643): n is a, c, g, or t
cctaaacgtcagtgcttgtggaactgctggcacgcaagtttcctnggggcggcctgaggaggagtaccgtcaccagctgggtcggtatggcatctccggagaactggccatgcgtcctcttgccagcctgtctgggggccagaagagccgagtggcctttgctcagatgactattgccctgccccacttctacattctggatgaacccacaaaccacctggacatggagaccattgaggctctgggccgtngctncaacaatttcaggggtggtgtgattctggtgtcccacgatgagcgctttatcaggctggtgtgccgggagttgtgggtatgcgaaggaggcggcgtcacccgtgtggaaggaggatttgaccagtaccgcgccctcctccagggaacagttccgccgcgaaggctttcctctagggccaccaggctgaggactcgccccaggacatggactggtctctcagacccctgggccaccatgtaggccaccantcccaggcnttggacttccccccaacttggggacagccttattcccaaatgtctctatccttttgactggagcatcttctgcacaaccttgggagcccatccaagggttggtgaggactggtctcccgggggtgggggnttggggggtacctctggggttatagattcccccactgccccagctctgactggaccccaagtggctgctatgtaaa

SEQ ID NO: 43 - DNA, 602 bp, Homo sapiens
cgtctagaatcgaggaggcaagcctgtgcccgaccgacgacacagagactcttctgatccaacccctagaaccgcgttgggtttgtgggtgtctcgtgctcagccactctgcccagctgggttggatcttctctccattcccctttctagctttaactaggaagatgtaggcagattggtggttttttttttttttttaacatacagaattttaaataccacaactggggcagaatttaaagctgcaacacagctggtgatgagaggcttcctcagtccagtcgctccttagcaccaggcaccgtgggtcctggatggggaactgcaagcagcctctcagctgatggctgcacagtcagatgtctggtggcagagagtccgagcatggagcgattccattttatgactgttgtttttcacattttcatctttctaaggtgtgtctcttttccaatgagaagtcatttttgcaagccaaaagtcgatcaatcgcattcattttaagaaattatacctttttagtacttgctgaagaatgattcagggtaaatcacatactttgtttagagaggcgaggggtttaacccgagtcacccagctgg

SEQ ID NO: 44 - DNA, 624 bp, Homo sapiens
cagtacttcagcattccacgatatggatttacggctttgcagcataatgaatttttgggacaaaacttctgcccaggactcaatgcaacaggaaacaatccttgtaactatgcaacatgtactggcgaagaatatttggtaaagcagggcatcgatctctcaccctggggcttgtggaagaatcacgtggccttggcttgtatgattgttattttcctcacaattgcctacctgaaattgttatttcttaaaaaatattcttaaatttccccttaattcagtatgatttatcctcacataaaaaagaagcactttgattgaagtattcaatcaagtttttttgttgttttctgttcccttgccatcacactgttgcacagcagcaattgttttaaagagatacatttttagaaatcacaacaaactgaattaaacatgaaagaacccaagacatcatgtatcgcatattagttaatctcctcagacagtaaccatggggaagaaatctggtctaatttattaatctaaaaaaggagaattgaattctggaaactcctgacaagttattactgtctctggcatttgtttcctcatctttaaaatgaataggtaggttagtagccc

SEQ ID NO: 45 - DNA, 585 bp, Homo sapiens
acaggcacatacatgagaacaggccatctcagccctacacacttgccatcccctacagcacagaggaagagtgatggtggcatgctggtggtggcgggtgctggtgggaggacagtgccaacctcctcctggggatcccatgttggagactctaaggataaggctggtgctgcccagggtgtctacaggaactgcaggtgtctacccccaagtcttccctcctcccaagccaggggtggcacagggcactagatccctggagttcaggaaccaacacaagcacaaccacgggcataagttggccttggccactgccacccacggccctccttttgtgctccatgctggcatcttcactcccctaccccttccccagccactgctgctcattcaaacttctgtccatgtccctccactgttcctatcagcaggtggcccctgggcatcagaacagcctgccctgggcaccaggtggcagacacactcagagcatgtctggctttcctggtgggtccaggctcattctgcttctgatttcccctcccccagggctcattttccccctttttcctgtacacatccctg

SEQ ID NO: 46 - DNA, 637 bp, Homo sapiens
gcccaggtgcaacatctagattcacaatgaactttctgattttgtattcatttattccagctcttgtcatcctaggaatagttgttttcaaaataagggatcatctcattagcaggtagtgaaagccatggctgggaaaatggaagtgaagctgccgactgtgcatgactgctctgaacgtctgaaatgagagtgccatgtatttctttcttgacaggacatctcaagtcttttaaccattaagactccatttgtgcctcttggatccaagcaggccttgaatgcaatggaagtggtttatagtcccttgctcttacaacttgcagggacatgtggttatttggaaattgtgactgagcggacccaagaatgtaaataatattcataaacctatgggagactcgtgtgactattttttttccttgttctaggcacagaaaaaaataggtcagcttaaaaatatgtttacattggataaaggattaggcaaaaataaaatgtttcaaggattcctgaccataagtgacagagaaagagagttgtgggtttagatgaagcaaggttatcatgcagaattgggtaagaatgcttctgttcctggaagacccagagttaaatgcagatgtccacacgaggg

SEQ ID NO: 47 - DNA, 698 bp, Homo sapiens
tcaatgaccatcggcttcctctattttggccatgggagcatccagctctccttcatggatacagccgccctcttgttcatgatcggtgctctcatccctttcaacgtcattctggatgtcatctccaaatgttactcagagagggcaatgctttactatgaactggaagacgggctgtacaccactggtccatatttctttgccaagatcctcggggagcttccggagcactgtgcctacatcatcatctacgggatgcccacctactggctggccaacctgaggccaggcctccagcccttcctgctgcacttcctgctggtgtggctggtggtcttctgttgcaggattatggccctggccgccgcggccctgctccccaccttccacatggcctccttcttcagcaatgccctctacaactccttctacctcgccgggggcttcatgataaacttgagcagcctgtggacagtgcccgcgtggatttccaaagtgtccttcctgcggtggtgttttgaagggctgatgaagattcagttcagcagaagaacttataaaatgcctctcgggaacctcaccatcgcggtctcaggagataaaatcctcagtgtcatggagctggactcgtaccctctctacgccatctacctcatcgtcattggcctcagcggtggcttcatggtcctgtactacgt

SEQ ID NO: 48 - DNA, 24 bp, Artificial Sequence
  primer
ccctgtggaatgtacctatgtgag

SEQ ID NO: 49 - DNA, 24 bp, Artificial Sequence
  primer
gcgtaaagtgcttggaatgagggc

SEQ ID NO: 50 - DNA, 24 bp, Artificial Sequence
  primer
ccttcaacacggacacgctctgct

SEQ ID NO: 51 - DNA, 24 bp, Artificial Sequence
  primer
agcttctccattcctgccacctgc

SEQ ID NO: 52 - DNA, 24 bp, Artificial Sequence
  primer
aaggaaaagtacggcgtggacgac

SEQ ID NO: 53 - DNA, 24 bp, Artificial Sequence
  primer
ctaagaccccagcacctaatcaca

SEQ ID NO: 54 - DNA, 24 bp, Artificial Sequence
  primer
gagcatcatcagaaaagggagggc

SEQ ID NO: 55 - DNA, 24 bp, Artificial Sequence
  primer
gggtttctagttctggggtctgga

SEQ ID NO: 56 - DNA, 24 bp, Artificial Sequence
  primer
aatgcaagccgtcaggaaagtttt

SEQ ID NO: 57 - DNA, 24 bp, Artificial Sequence
  primer
cttacacttcagcttttacggatg

SEQ ID NO: 58 - DNA, 24 bp, Artificial Sequence
  primer
agttgtgttttgtgctgagcctcc

SEQ ID NO: 59 - DNA, 24 bp, Artificial Sequence
  primer
gtgcctgactctttgggtgacttt

SEQ ID NO: 60 - DNA, 24 bp, Artificial Sequence
  primer
atagcatggaggagtgtgaagcgc

SEQ ID NO: 61 - DNA, 24 bp, Artificial Sequence
  primer
tttcaccaccacggcttctctcca

SEQ ID NO: 62 - DNA, 24 bp, Artificial Sequence
  primer
gctgggtgattttgaggaggattt

SEQ ID NO: 63 - DNA, 24 bp, Artificial Sequence
  primer
gaaaatggcacacagttggcttac

SEQ ID NO: 64 - DNA, 24 bp, Artificial Sequence
  primer
tgtgccagcaaccaaatcccatgt

SEQ ID NO: 65 - DNA, 24 bp, Artificial Sequence
  primer
tttctcctaatgctatccctcccc

SEQ ID NO: 66 - DNA, 24 bp, Artificial Sequence
  primer
aggagctgggaaatgttgatgata

SEQ ID NO: 67 - DNA, 24 bp, Artificial Sequence
  primer
gccatttcatcagtttatcagacc

SEQ ID NO: 68 - DNA, 24 bp, Artificial Sequence
  primer
cctgctggagagtgttttgggctt

SEQ ID NO: 69 - DNA, 24 bp, Artificial Sequence
  primer
atgtttgcgactcctcctgctgtg

SEQ ID NO: 70 - DNA, 24 bp, Artificial Sequence
  primer
catcctgtttgactgcagcattgc

SEQ ID NO: 71 - DNA, 24 bp, Artificial Sequence
  primer
gcaaggcagtcagttacagtccaa

SEQ ID NO: 72 - DNA, 24 bp, Artificial Sequence
  primer
atattgcctatggcctgacccaga

SEQ ID NO: 73 - DNA, 24 bp, Artificial Sequence
  primer
ttctcagtttcagagtgctggcca

SEQ ID NO: 74 - DNA, 24 bp, Artificial Sequence
  primer
gggagtaggagctatgctaagtgt

SEQ ID NO: 75 - DNA, 24 bp, Artificial Sequence
  primer
tgctcatggtctagtggaaggtca

SEQ ID NO: 76 - DNA, 24 bp, Artificial Sequence
  primer
ttgacagctacagtgaagaggggc

SEQ ID NO: 77 - DNA, 24 bp, Artificial Sequence
  primer
cataagttctgtgtcccagcctgg

SEQ ID NO: 78 - DNA, 24 bp, Artificial Sequence
  primer
ttcgcttctacgacatcagctctg

SEQ ID NO: 79 - DNA, 24 bp, Artificial Sequence
  primer
gaccaggatgaaataagccaggga

SEQ ID NO: 80 - DNA, 24 bp, Artificial Sequence
  primer
ccctgcaggaaagaaagtggccat

SEQ ID NO: 81 - DNA, 24 bp, Artificial Sequence
  primer
cttagcacgaacagtttccacagc

SEQ ID NO: 82 - DNA, 24 bp, Artificial Sequence
  primer
aggttgtcggtttcatcagccagg

SEQ ID NO: 83 - DNA, 24 bp, Artificial Sequence
  primer
tttattgtgagcaggagcagccgc

SEQ ID NO: 84 - DNA, 24 bp, Artificial Sequence
  primer
tggatcaccgcttcctgcatcttg

SEQ ID NO: 85 - DNA, 24 bp, Artificial Sequence
  primer
tgccaccatcccatccaccaaaga

SEQ ID NO: 86 - DNA, 24 bp, Artificial Sequence
  primer
gcaaggcatgaactgctaggtatt

SEQ ID NO: 87 - DNA, 24 bp, Artificial Sequence
  primer
ggtttcttcttccagtctaatcag

SEQ ID NO: 88 - DNA, 24 bp, Artificial Sequence
  primer
ttgtcattgcccatcgcttgtcca

SEQ ID NO: 89 - DNA, 24 bp, Artificial Sequence
  primer
agagcatccaccctttccctatcc

SEQ ID NO: 90 - DNA, 24 bp, Artificial Sequence
  primer
gctcccatcacctctaacatcctt

SEQ ID NO: 91 - DNA, 24 bp, Artificial Sequence
  primer
tgagcaggtaccatgagagggaaa

SEQ ID NO: 92 - DNA, 24 bp, Artificial Sequence
  primer
gtagcatggagaagattggtgtgg

SEQ ID NO: 93 - DNA, 24 bp, Artificial Sequence
  primer
gggtagtaggttcatgggtgttca

SEQ ID NO: 94 - DNA, 24 bp, Artificial Sequence
  primer
caagagccgcatcctggttttaga

SEQ ID NO: 95 - DNA, 24 bp, Artificial Sequence
  primer
tttaatggattcaggcagcacccc

SEQ ID NO: 96 - DNA, 24 bp, Artificial Sequence
  primer
tgggaagaaccggagctggaaaaa

SEQ ID NO: 97 - DNA, 24 bp, Artificial Sequence
  primer
aatgccttcggaacggacttgaca

SEQ ID NO: 98 - DNA, 24 bp, Artificial Sequence
  primer
aaggaagacgtgtggcaatagtgg

SEQ ID NO: 99 - DNA, 24 bp, Artificial Sequence
  primer
aaaccacacagcaaccagcaacct

SEQ ID NO: 100 - DNA, 24 bp, Artificial Sequence
  primer
tcgtgtcagtggagcggatgcagg

SEQ ID NO: 101 - DNA, 24 bp, Artificial Sequence
  primer
ctgccacctgccccttgtccatga

SEQ ID NO: 102 - DNA, 24 bp, Artificial Sequence
  primer
tctttcacaggggacaggatggtt

SEQ ID NO: 103 - DNA, 24 bp, Artificial Sequence
  primer
cagtttggagttgagaaggcagtg

SEQ ID NO: 104 - DNA, 24 bp, Artificial Sequence
  primer
aaaccgaggcagagagctacgagg

SEQ ID NO: 105 - DNA, 24 bp, Artificial Sequence
  primer
tgggctctggcaggtcacttgtct

SEQ ID NO: 106 - DNA, 24 bp, Artificial Sequence
  primer
tgggtgcagtgaagaaggtgaaca

SEQ ID NO: 107 - DNA, 24 bp, Artificial Sequence
  primer
gtgggcgaacaaatttgggacagt

SEQ ID NO: 108 - DNA, 24 bp, Artificial Sequence
  primer
tcttccctgttgttggtgctcttc

SEQ ID NO: 109 - DNA, 24 bp, Artificial Sequence
  primer
tgaaaatgcaagtgggctcctatg

SEQ ID NO: 110 - DNA, 24 bp, Artificial Sequence
  primer
gattctcattgacggcgtggacat

SEQ ID NO: 111 - DNA, 24 bp, Artificial Sequence
  primer
tggttctggggttctaaggtcttg

SEQ ID NO: 112 - DNA, 24 bp, Artificial Sequence
  primer
ctggttatggaaaatgggaaggtg

SEQ ID NO: 113 - DNA, 24 bp, Artificial Sequence
  primer
ttgcaaggcgacatttcagggtaa

SEQ ID NO: 114 - DNA, 24 bp, Artificial Sequence
  primer
gcacctgtgggccatactaaaaga

SEQ ID NO: 115 - DNA, 24 bp, Artificial Sequence
  primer
taacaaacacaaggactgccaccc

SEQ ID NO: 116 - DNA, 24 bp, Artificial Sequence
  primer
ttccctcctcgtcagtctctcaaa

SEQ ID NO: 117 - DNA, 24 bp, Artificial Sequence
  primer
tctttggcactgagctgggaacat

SEQ ID NO: 118 - DNA, 24 bp, Artificial Sequence
  primer
gtggccaactaaacctgtacaaaa

SEQ ID NO: 119 - DNA, 24 bp, Artificial Sequence
  primer
acaaaagagcactaaaccagagag

SEQ ID NO: 120 - DNA, 24 bp, Artificial Sequence
  primer
tactcattccttgtgtgtgtcttg

SEQ ID NO: 121 - DNA, 24 bp, Artificial Sequence
  primer
cttcggtagccagtgattgttata

SEQ ID NO: 122 - DNA, 24 bp, Artificial Sequence
  primer
ctccatatgcttgaagtgctgatt

SEQ ID NO: 123 - DNA, 24 bp, Artificial Sequence
  primer
agaagcctggcaaacattatgaag

SEQ ID NO: 124 - DNA, 24 bp, Artificial Sequence
  primer
attccccgcaaaaaacccctaact

SEQ ID NO: 125 - DNA, 24 bp, Artificial Sequence
  primer
tgggagggtaataaagggagatca

SEQ ID NO: 126 - DNA, 24 bp, Artificial Sequence
  primer
ttggaggccctgggtgaagtcatg

SEQ ID NO: 127 - DNA, 24 bp, Artificial Sequence
  primer
tttcctgccccaagtcctcaacca

SEQ ID NO: 128 - DNA, 24 bp, Artificial Sequence
  primer
tgctacccagagatcaaggagaag

SEQ ID NO: 129 - DNA, 24 bp, Artificial Sequence
  primer
acttggagctggtgtacttggtga

SEQ ID NO: 130 - DNA, 24 bp, Artificial Sequence
  primer
cctaaacgtcagtgcttgtggaac

SEQ ID NO: 131 - DNA, 24 bp, Artificial Sequence
  primer
tttacatagcagccacttggggtc

SEQ ID NO: 132 - DNA, 24 bp, Artificial Sequence
  primer
cgtctagaatcgaggaggcaagcc

SEQ ID NO: 133 - DNA, 24 bp, Artificial Sequence
  primer
ccagctgggtgactcgggttaaac

SEQ ID NO: 134 - DNA, 24 bp, Artificial Sequence
  primer
cagtacttcagcattccacgatat

SEQ ID NO: 135 - DNA, 24 bp, Artificial Sequence
  primer
gggctactaacctacctattcatt

SEQ ID NO: 136 - DNA, 24 bp, Artificial Sequence
  primer
acaggcacatacatgagaacaggc

SEQ ID NO: 137 - DNA, 24 bp, Artificial Sequence
  primer
cagggatgtgtacaggaaaaaggg

SEQ ID NO: 138 - DNA, 24 bp, Artificial Sequence
  primer
gcccaggtgcaacatctagattca

SEQ ID NO: 139 - DNA, 24 bp, Artificial Sequence
  primer
ccctcgtgtggacatctgcattta

SEQ ID NO: 140 - DNA, 24 bp, Artificial Sequence
  primer
tcaatgaccatcggcttcctctat

SEQ ID NO: 141 - DNA, 24 bp, Artificial Sequence
  primer
acgtagtacaggaccatgaagcca